SEQUENCE LISTING (24 sequences)

SEQ ID NO: 1 - DNA, 25 bp, Artificial Sequence
  Control-sgRNA-F
caccggcactaccagagctaactca

SEQ ID NO: 2 - DNA, 25 bp, Artificial Sequence
  Control-sgRNA-R
aaactgagttagctctggtagtgcc

SEQ ID NO: 3 - DNA, 25 bp, Artificial Sequence
  DHODH-sgRNA#2-F
caccgcatcttataaagtccgtcca

SEQ ID NO: 4 - DNA, 25 bp, Artificial Sequence
  DHODH-sgRNA#2-R
aaactggacggactttataagatgc

SEQ ID NO: 5 - DNA, 25 bp, Artificial Sequence
  DHODH-sgRNA#4-F
caccgtgagttgataaatcccggag

SEQ ID NO: 6 - DNA, 25 bp, Artificial Sequence
  DHODH-sgRNA#4-R
aaacctccgggatttatcaactcac

SEQ ID NO: 7 - DNA, 25 bp, Artificial Sequence
  ACSL4-sgRNA-F
caccgtgcaatcatccattcggccc

SEQ ID NO: 8 - DNA, 25 bp, Artificial Sequence
  ACSL4-sgRNA-R
aaacgggccgaatggatgattgcac

SEQ ID NO: 9 - DNA, 25 bp, Artificial Sequence
  FSP1-sgRNA-F
caccgggagatggggtcccaggtct

SEQ ID NO: 10 - DNA, 25 bp, Artificial Sequence
  FSP1-sgRNA-R
aaacagacctgggaccccatctccc

SEQ ID NO: 11 - DNA, 25 bp, Artificial Sequence
  COQ2-sgRNA-F
caccgatgctgggctcgcgagccgc

SEQ ID NO: 12 - DNA, 25 bp, Artificial Sequence
  COQ2-sgRNA-R
aaacgcggctcgcgagcccagcatc

SEQ ID NO: 13 - DNA, 29 bp, Artificial Sequence
  GPX4 sh#2
ccgctgtggaagtggatgaagatccaacc

SEQ ID NO: 14 - DNA, 29 bp, Artificial Sequence
  GPX4 sh#3
ggaaatgccatcaagtggaacttcaccaa

SEQ ID NO: 15 - DNA, 22 bp, Artificial Sequence
  SLC7A11-F
tcctgctttggctccatgaacg

SEQ ID NO: 16 - DNA, 22 bp, Artificial Sequence
  SLC7A11-R
agaggagtgtgcttgcggacat

SEQ ID NO: 17 - DNA, 22 bp, Artificial Sequence
  GPX4-F
acaagaacggctgcgtggtgaa

SEQ ID NO: 18 - DNA, 22 bp, Artificial Sequence
  GPX4-R
gccacacacttgtggagctaga

SEQ ID NO: 19 - DNA, 22 bp, Artificial Sequence
  ACSL4-F
gctatctcctcagacacaccga

SEQ ID NO: 20 - DNA, 22 bp, Artificial Sequence
  ACSL4-R
aggtgctccaactctgccagta

SEQ ID NO: 21 - DNA, 22 bp, Artificial Sequence
  PTGS2-F
cggtgaaactctggctagacag

SEQ ID NO: 22 - DNA, 22 bp, Artificial Sequence
  PTGS2-R
gcaaaccgtagatgctcaggga

SEQ ID NO: 23 - DNA, 22 bp, Artificial Sequence
  ACTB-F
caccattggcaatgagcggttc

SEQ ID NO: 24 - DNA, 22 bp, Artificial Sequence
  ACTB-R
aggtctttgcggatgtccacgt

(19) United States
(12) Patent Application Publication
(10) Pub. No.: US 2024/0148757 A1
(43) Pub. Date: May 9, 2024

(54) USE OF DIHYDROOROTATE DEHYDROGENASE (DHODH) INHIBITORS TO TARGET FERROPTOSIS IN CANCER THERAPY

(71) Applicants: BOARD OF REGENTS, THE UNIVERSITY OF TEXAS SYSTEM, Austin, TX (US); KADMON CORPORATION LLC, New York, NY (US)

(72) Inventors: Boyi GAN, Houston, TX (US); Kellen OLSZEWSKI, Brooklyn, NY (US); Chao MAO, Houston, TX (US)

(21) Appl. No.: 18/548,678

(22) PCT Filed: Mar. 3, 2022
(86) PCT No.: PCT/US2022/018663

§ 371 Date: Sep. 1, 2023

Related U.S. Application Data
(60) Provisional application No. 63/156,179, filed on Mar. 3, 2021.

(51) Int. Cl. A61K 31/655; A61K 31/18; A61K 31/277; A61K 31/381; A61K 31/42; A61K 31/437; A61K 31/47; A61K 31/517; A61P 35/00; C12Q 1/6851; C12Q 1/6886; G01N 33/574

(52) U.S. Cl. CPC A61K 31/655; A61K 31/18; A61K 31/277; A61K 31/381; A61K 31/42; A61K 31/437; A61K 31/47; A61K 31/517; A61P 35/00; C12Q 1/6851; C12Q 1/6886; G01N 33/57496; C12Q 2600/106; C12Q 2600/158; G01N 2333/90206

(57) ABSTRACT

The present disclosure provides methods for treating cancer comprising administering to a subject in need thereof a therapeutically effective amount of a dihydroorotate dehydrogenase (DHODH) inhibitor, wherein the cancer has an altered glutathione peroxidase 4 (GPX4) expression as compared to a control sample. The present disclosure also provides methods of treating a subject with a dihydroorotate dehydrogenase (DHODH) inhibitor where the subject's GPX4 expression is low as compared to a control sample or with a DHODH inhibitor and a ferroptosis inducer where the subject's GPX4 expression is high as compared to a control sample.

### CROSS REFERENCE TO RELATED APPLICATIONS

The present application claims the priority benefit of U.S. Provisional Application No. 63/156,179, filed Mar. 3, 2021, which is hereby incorporated by reference in its entirety.

### GOVERNMENTAL RIGHTS

This invention was made with government support under grant number CA181196 awarded by the National Institutes of Health. The government has certain rights in the invention.

### INCORPORATION BY REFERENCE OF SEQUENCE LISTING

The content of the electronically submitted sequence listing (Name: 4443_001PC01_Seqlisting_ST25; Size: 4,812 bytes; and Date of Creation: Feb. 24, 2022) filed with the application is incorporated herein by reference in its entirety.

### BACKGROUND

Ferroptosis is a form of non-apoptotic cell death induced by excessive lipid peroxidation (Dixon, S. J., et al., Cell 149, 1060-1072 (2012); Stockwell, B. R., et al., Cell 171, 273-285 (2017)). Cells have evolved at least two defense mechanisms to suppress ferroptosis. In the first, glutathione peroxidase 4 (GPX4) utilizes reduced glutathione (GSH) to detoxify lipid hydroperoxides and inhibit ferroptosis (Jiang, L., et al., Nature 520, 57-62 (2015); Zhang, Y., et al., Nat Cell Biol 20, 1181-1192 (2018)). Most cancer cells rely on solute carrier family 7 member 11 (SLC7A11)-mediated cystine transport to obtain cysteine for GSH biosynthesis (Yang, W. S., et al., Cell 156, 317-331 (2014)). The SLC7A11-GPX4 signaling axis represents the major cellular defense system against ferroptosis, and inactivation of GPX4 or SLC7A11 by corresponding ferroptosis inducers induces ferroptosis in many cancer cells (Dixon, S. J., et al., Cell 149, 1060-1072 (2012) Jiang, L., et al., Nature 520, 57-62 (2015); Zhang, Y., et al., Nat Cell Biol 20, 1181-1192 (2018)). In the second, ferroptosis suppressor protein 1 (FSP1; also called AIFM2) acts as another ferroptosis inhibitor that acts in parallel to GPX4 to suppress ferroptosis. Mechanistically, FSP1 functions as an oxidoreductase primarily localized on the plasma membrane to reduce ubiquinone (CoQ) to ubiquinol (CoQH₂), which then acts as a lipophilic radical trapping antioxidant (RTA) to detoxify lipid hydroperoxides (Friedmann Angeli, J. P., et al., Nat Cell Biol 16, 1180-1191 (2014); Bersuker, K., et al., Nature 575, 688-692 (2019)). Whether there exist additional cellular defense mechanisms against ferroptosis at other subcellular compartments remains unclear.

Ferroptosis has recently emerged as a critical tumor suppression mechanism (Dixon, S. J., et al., Cell 149, 1060-1072 (2012); Stockwell, B. R., et al., Cell 171, 273-285 (2017); Doll, S., et al., Nature 575, 693-698 (2019); Koppula, P., Zhuang, L. & Gan, B., Protein Cell (2020)). However, the ability to target ferroptosis in cancer therapy is hindered by an incomplete understanding of ferroptosis mechanisms. In addition, despite an emerging understanding of the role of ferroptosis in tumor suppression, there is a critical need to identify the specific context for therapeutic targeting of ferroptosis and/or rational drug combination strategies.

### SUMMARY

As described herein, dihydroorotate dehydrogenase (DHODH) is a ferroptosis defense mechanism that, among other things, operates in mitochondria and independent of GPX4 and FSP1. DHODH inhibition provides a means for therapeutic targeting of ferroptosis and rational drug combination design. And as described herein, an array of GPX4^{low} solid tumors can be effectively targeted by DHODH inhibitors to reduce tumor burden, and a combination of DHODH inhibitors with ferroptosis inducers, including but not limited to sulfasalazine, can be used to treat GPX4^{high} solid tumors.

The present disclosure provides a method for treating cancer, the method comprising administering to a subject in need thereof a therapeutically effective amount of a dihydroorotate dehydrogenase (DHODH) inhibitor, wherein the cancer has an altered glutathione peroxidase 4 (GPX4) expression as compared to a control sample.

In aspects of the methods described herein, the altered GPX4 expression is a low expression level of GPX4 as compared to a control sample. In some aspects, the cancer is a tumor. In some aspects, the tumor is a carcinoma. In some aspects, the cancer is relapsed, refractory, or refractory following at least one prior therapy comprising administration of at least one anticancer agent. In some aspects, the cancer is selected from fibrosarcoma, lung squamous cell carcinoma, lung adenocarcinoma, renal cell carcinoma, breast adenocarcinoma, colorectal adenocarcinoma, endocervical adenocarcinoma, or T acute lymphoblastic leukemia.

In some aspects, the DHODH inhibitor is selected from the group consisting of Ag-636, ASLAN003, BAY2402234, leflunomide, brequinar, teriflunomide, IMU-838, PP-001, PTC299, and combinations thereof. In some aspects, the DHODH inhibitor is selected from the group consisting of leflunomide, brequinar, teriflunomide, and combinations thereof.

In aspects of the methods described herein, the altered GPX4 expression is a high expression level of GPX4 as compared to a control sample. In some aspects, the cancer is a tumor. In some aspects, the tumor is a carcinoma. In some aspects, the cancer is relapsed, refractory, or refractory following at least one prior therapy comprising administration of at least one anticancer agent. In some aspects, the cancer is selected from fibrosarcoma, lung squamous cell carcinoma, lung adenocarcinoma, renal cell carcinoma, breast adenocarcinoma, colorectal adenocarcinoma, endocervical adenocarcinoma, or T acute lymphoblastic leukemia.

In aspects of the methods described herein, the method further comprises administering a therapeutically effective amount of a ferroptosis inducer. In some aspects, the ferroptosis inducer is a class I ferrotosis inducer, a class II ferroptosis inducer, or a combination thereof. In some aspects, the ferroptosis inducer is selected from the group consisting of sulfasalazine, rosiglitazone, rosiglitazone maleate, bardoxolone methyl, linagliptin, curcumin, zileuton, pioglitazone HCl, nordihydroguaiaretic acid (NDGA), troglitazone, setanaxib, deferoxamine mesylate, sorafenib tosylate, cisplatin, rosadustat, lapatinib, simvastatin, deferasirox, sorafenib, erastin, imidazole ketone erastin, RSL3, (1S,3R)-RSL3, ML210, ML162, and combinations thereof. In some aspects, the ferroptosis inducer is selected from the group consisting of sulfasalazine, sorafenib tosylate, erastin, imidazole ketone erasin, and combinations thereof.

In some aspects, the subject is a human.

The present disclosure also provides a method of treating a subject with a dihydroorotate dehydrogenase (DHODH) inhibitor, wherein the subject is afflicted with a cancer, the method comprising (a) determining, in the cancer sample, the expression level of glutathione peroxidase 4 (GPX4) and (b) if the expression level of GPX4 is low as compared to relative GPX4 expression level in a control sample, then administering a therapeutically effective amount of the DHODH inhibitor to the subject, or (c) if the expression level of GPX4 is high as compared to relative GPX4 expression level in a control sample, then administering a therapeutically effective amount of the DHODH inhibitor and a therapeutically effective amount of a ferroptosis inducer to the subject.

The present disclosure also provides a method of treating a subject afflicted with a cancer, comprising administering to the subject a therapeutically effective amount of a dihydroorotate dehydrogenase (DHODH) inhibitor, wherein, prior to the administration, the subject is identified as exhibiting an altered expression level of glutathione peroxidase 4 (GPX4) as compared to relative GPX4 expression level in a control sample.

In some aspects, if the subject is identified as exhibiting a low expression level of GPX4, the treatment comprises administering a therapeutically effective amount of the DHODH inhibitor to the subject. In some aspects, if the subject is identified as exhibiting a high expression level of GPX4, the treatment comprises administering a therapeutically effective amount of the DHODH inhibitor and a therapeutically effective amount of a ferroptosis inducer to the subject.

In some aspects, identifying the subject as exhibiting an altered expression level of GPX4 comprises obtaining a cancer sample from the subject and analyzing the sample for the GPX4 expression level.

The present disclosure also provides a method of identifying a subject afflicted with a cancer as suitable for treatment with a dihydroorotate dehydrogenase (DHODH) inhibitor, the method comprising determining whether the subject has an altered expression level of glutathione peroxidase 4 (GPX4) as compared to relative GPX4 expression level in a control sample, wherein (a) if the expression level of GPX4 is low as compared to relative GPX4 expression level in a control sample, then a therapeutically effective amount of the DHODH inhibitor can be administered to the subject, or (b) if the expression level of GPX4 is high as compared to relative GPX4 expression level in a control sample, then a therapeutically effective amount of the DHODH inhibitor and a therapeutically effective amount of a ferroptosis inducer can be administered to the subject.

In some aspects, determining whether the subject has an altered expression level of GPX4 comprises obtaining a cancer sample from the subject and analyzing the sample for the GPX4 expression level.

In some aspects, the dihydroorotate dehydrogenase (DHODH) inhibitor is selected from the group consisting of Ag-636, ASLAN003, BAY2402234, leflunomide, brequinar, teriflunomide, IMU-838, PP-001, PTC299, and combinations thereof. In some aspects, the dihydroorotate dehydrogenase (DHODH) inhibitor is selected from the group consisting of leflunomide, brequinar, teriflunomide, and combinations thereof.

In some aspects, the ferroptosis inducer is a class I ferrotosis inducer, a class II ferroptosis inducer, or a combination thereof. In some aspects, the ferroptosis inducer is selected from the group consisting of sulfasalazine, rosiglitazone, rosiglitazone maleate, bardoxolone methyl, linagliptin, curcumin, zileuton, pioglitazone HCl, nordihydroguaiaretic acid (NDGA), troglitazone, setanaxib, deferoxamine mesylate, sorafenib tosylate, cisplatin, rosadustat, lapatinib, simvastatin, deferasirox, sorafenib, erastin, imidazole ketone erastin, RSL3, (1S,3R)-RSL3, ML210, ML162, and combinations thereof. In some aspects, the ferroptosis inducer is selected from the group consisting of sulfasalazine, sorafenib tosylate, erastin, imidazole ketone erasin, and combinations thereof.

In some aspects, the subject is a human.

In aspects of the methods described herein, the cancer sample comprises tumor tissue, intratumoral tissue, blood sample, bone marrow, or combinations thereof.

In aspects of the methods described herein, the GPX4 expression levels are determined using sequencing or any technology that measures RNA or protein expression level. In some aspects, the GPX4 expression levels are determined by PCR, real-time PCR, deep sequencing, Next Generation Sequencing (NGS), RNA-Seq, EdgeSeq, PCR, Nanostring, microarray expression profiling, immunohistochemical methods, ELISA, Western analysis, HPLC, proteomics assays, or a combination thereof.

In aspects of the methods described herein, the methods described herein further comprise a. administering chemotherapy; b. performing surgery; c. administering radiation therapy; d. administering immunotherapy; e. administering targeted therapy; or f. any combination thereof.

In some aspects, administering the therapeutically effective amount of a dihydroorotate dehydrogenase (DHODH) inhibitor or both the therapeutically effective amount of a dihydroorotate dehydrogenase (DHODH) inhibitor and the therapeutically effective amount of a ferroptosis inducer reduces the cancer burden.

### BRIEF DESCRIPTION OF THE DRAWINGS

FIG. 1A, FIG. 1B, FIG. 1C, FIG. 1D, FIG. 1E, FIG. 1F, FIG. 1G, FIG. 1H, FIG. 1I, FIG. 1J, FIG. 1K, FIG. 1L, and FIG. 1M show DHODH inactivation promotes ferroptosis in GPX4^{low} cancer cells. FIG. 1A, and FIG. 1B are volcano plots comparing metabolomic profiles from HT-1080 cells treated with vehicle and those treated with RSL3 (10 μM) (FIG. 1A) or ML162 (10 μM) (FIG. 1B) for 2 hours. n=3 independent experiments. FIG. 1C and FIG. 1D are bar graph showing the fold changes in C-Asp (FIG. 1C) and uridine (FIG. 1D) induced by RSL3 (10 μM) or ML162 (10 μM) treatment for 2 hours in HT-1080 cells. Error bars are mean±s.d, n=3 independent repeats. FIG. 1E is a simplified schematic of de novo pyrimidine biosynthesis pathway. FIG. 1F and FIG. 1G are graphs showing cell viability measurement in NCI-H226 (FIG. 1F) or HT-1080 cells (FIG. 1G) with different doses of RSL3 treatment for 4 hours following pretreatment with vehicle, C-Asp (100 μM), DHO (100 μM), OA (100 μM), or uridine (50 μM) for 48 hours. FIG. 1H and FIG. 1I are graphs showing cell viability measurement in NCI-H226 (FIG. 1H) or HT-1080 cells (FIG. 1I) treated with different doses of BQR for 4 hours following pretreatment with Lip-1 (10 μM) or ZVF (10 μM) for 24 hours. FIG. 1J and FIG. 1K are graphs showing lipid peroxidation measurement upon 4-hour treatment with BQR in NCI-H226 (500 μM) (FIG. 1I) or HT-1080 (5 mM) (FIG. 1K) cells following pretreatment with Lip-1 (10 μM) or ZVF (10 μM) for 24 hours. Error bars are mean±s.d, n=3 independent repeats. FIG. 1L is a graph showing cell viability measurement in HT-1080 cells with different doses of RSL3 and co-treatment with BQR (500 μM) for 4 hours. FIG. 1M is a graph showing lipid peroxidation measurement in HT-1080 cells treated with RSL3 (1 μM) and/or BQR (500 μM) for 4 hours. Error bars are mean±s.d, n=3 independent repeats. Asp, aspartate; C-P, carbamoyl phosphate; P, phosphate; C-Asp, N-Carbamoyl-L-aspartate; DHO, dihydroorotate; FMN, flavin mononucleotide; FMNH₂, reduced flavin mononucleotide; OA, orotate; PRPP, phosphoribosyl pyrophosphate; PPi, inorganic pyrophosphate; OMP, orotidine 5′-monophosphate; CO₂, carbon dioxide; UMP, uridine 5′-monophosphate; BQR, brequinar; Lip-1, liproxstatin-1; ZVF, N-benzyloxycarbonyl-val-ala-asp(o-me) fluoromethyl ketone.

FIG. 2A, FIG. 2B, FIG. 2C, FIG. 2D, FIG. 2E, FIG. 2F, FIG. 2G, FIG. 2H, and FIG. 2I show the effect of DHODH inhibitors on inducing ferroptosis in different cancer cells with differential expression of GPX4. FIG. 2A, and FIG. 2B are graphs showing measurement of cell survival fraction and PTGS2 mRNA levels in NCI-H226 (FIG. 2A) or HT-1080 (FIG. 2B) cells upon treatment with BQR (50011M for NCI-H226 cells; 5 mM for HT-1080 cells), following pretreatment with vehicle, ZVF (10 μM), and/or Lip-1 (10 μM) for 24 hours. Error bars are mean±s.d, n=3 independent repeats. FIG. 2C are graphs showing cell viability measurement in HT-1080 cells treated with different doses of RSL3 and co-treatment with LFM (100 μM) or TF (500 μM) for 4 hours, following pretreatment with vehicle or Lip-1 (10 μM) for 24 hours. FIG. 2D is graphs showing cell viability measurement in HT-1080 cells treated with different doses of ML162 and co-treatment with BQR (500 μM), LFM (100 μM), or TF (500 μM) for 4 hours, following pretreatment with vehicle or Lip-1 (10 μM) for 24 hours. FIG. 2E is graphs showing measurement of cell survival fraction and PTGS2 mRNA levels in HT-1080 cells upon treatment with RSL3 (1 μM) and/or BQR (500 μM) for 4 hours, following pretreatment with vehicle or Lip-1 (10 μM) for 24 hours. Error bars are mean±s.d, n=3 independent repeats. FIG. 2F is graphs showing cell viability measurement in HT-1080 cells treated with different doses of SAS and co-treatment with BQR (500 μM), LFM (100 μM) or TF (500 μM) for 4 hours, following pretreatment with vehicle or Lip-1 (10 μM) for 24 hours. FIG. 2G is graphs showing cell viability measurement in HT-1080 cells treated with different doses of erastin and co-treatment with BQR (500 μM), LFM (100 μM) or TF (500 μM) for 4 hours, following pretreatment with vehicle or Lip-1 (10 μM) for 24 hours. FIG. 2H is graphs showing mRNA levels of SLC7A11, GPX4, or ACSL4, as well as their protein levels were measured in HT-1080 cells treated with BQR (500 μM), LFM (100 μM), or TF (500 μM) for 4 hours. Error bars are mean±s.d, n=3 independent repeats. FIG. 2I is a graph showing GSH level measurement in HT-1080 cells upon treatment with BQR (500 μM), LFM (100 μM), or TF (500 μM) for 4 hours. Error bars are mean±s.d, n=3 independent repeats. BQR, brequinar; LFM, leflunomide; TF, teriflunomide; ZVF, N-benzyloxycarbonyl-val-ala-asp(o-me) fluoromethyl ketone; Lip-1, liproxstatin-1; SAS, sulfasalazine; GSH, glutathione.

FIG. 3A, FIG. 3B, FIG. 3C, FIG. 3D, FIG. 3E, FIG. 3F, FIG. 3G, FIG. 3H, FIG. 3I, FIG. 3J, FIG. 3K, FIG. 3L, and FIG. 3M show pharmacologic inhibition of GPX4 affects intermediate levels in the de novo pyrimidine biosynthesis pathway. FIG. 3A and FIG. 3B are volcano plots comparing metabolomic profiles from A-498 (FIG. 3A) or RCC4 (FIG. 3B) cells treated with vehicle and the same cells treated with RSL3 (10 μM) or ML162 (10 μM) for 2 hours. n=3 independent repeats. FIG. 3C and FIG. 3D are graphs showing the fold changes in C-Asp and uridine induced by RSL3 (10 μM) or ML162 (10 μM) treatment for 2 hours compared with vehicle treatment in A-498 (FIG. 3C) or RCC4 (FIG. 3D) cells. Error bars are mean±s.d, n=3 independent repeats. FIG. 3E depicts a graph showing amide-⁵N-glutamine labeling analysis of ¹⁵N-UMP levels in HT-1080 cells upon treatment with RSL3 (10 μM) and/or Lip-1 (10 μM) for 2 hours. FIG. 3F is graphs showing the fold changes in intracellular DHO or OA levels upon treatment with vehicle, DHO (100 μM) or OA (100 μM), respectively, for 48 hours in NCI-H226 cells. FIG. 3G is a graph showing the fold changes in intracellular C-Asp levels upon treatment with vehicle or C-Asp (100 μM) for 48 hours in NCI-H226 cells. FIG. 3H is a graph that depicts DHO activity measurement in HT-1080 cells treated with RSL3 (10 μM) for 2 hours, following pretreatment with vehicle, OA (100 μM) for 24 hours, or Lip-1 (10 μM) for 48 hours. Error bars are mean±s.d, n=3 independent repeats. FIG. 3I is an image of GPX4 protein, DHODH, and FSP1 levels versus Vinculin in different cell lines as determined by western blotting. FIG. 3J is graphs that depicts cell viability measurement in TK-10, UMRC2, A498 or RCC4 cells treated with different doses of RSL3 for 4 hours, following pretreatment with vehicle, C-Asp (100 μM), DHO (100 μM), OA (100 μM), or uridine (50 μM) for 48 hours. FIG. 3K is graphs showing cell viability measurement in SW620, U-87 MG, A549, NCI-H1437, MDA-MB-436 or MDA-MB-231 cells treated with different doses of RSL3 for 4 hours, following pretreatment with vehicle, DHO (100 μM) or OA (100 μM) for 48 hours. FIG. 3L is an image of GPX4, DHODH, and FSP1 protein levels in different cancer cell lines as determined by western blotting. FIG. 3M is graphs depicting cell viability measurement in GPX4^{high} (HT-1080, A-498, RCC4, 786-O and 769-P) and GPX4^{low} (HCT-8, UMRC6, TK-10, UMRC2 and NCI-H226) cells treated with different doses of BQR, LFM, or TF for 4 hours. C-Asp, N-Carbamoyl-L-aspartate; UMP, uridine 5′-monophosphate; DHO, dihydroorotate; OA, orotate; BQR, brequinar; LFM, leflunomide; TF, teriflunomide.

FIG. 4A, FIG. 4B, FIG. 4C, FIG. 4D, FIG. 4E, FIG. 4F, FIG. 4G, FIG. 4H, and FIG. 4I show the genetic interaction between DHODH and GPX4 in regulating ferroptosis. FIG. 4A is a graph showing high DHODH expression correlates with resistance to GPX4 inhibitors (RSL3, ML162, and ML210) in cancer cells. Plotted data were mined from the CTRP database. Plotted values are Pearson's correlation coefficients. FIG. 4B is a graph showing cell viability measurement in Cas9^{ctrl} and DHODH^{ko} HT-1080 cells treated with different doses of RSL3 for 4 hours. Cells were grown in medium supplemented with uridine (50 μM). FIG. 4C is a graph showing lipid peroxidation measurement in Cas9^{ctrl} and DHODH^{ko} HT-1080 cells upon treatment with RSL3 (1 μM) for 4 hours. Cells were grown in medium supplemented with uridine (50 μM). Error bars are mean±s.d, n=3 independent repeats. FIG. 4D is a graph showing lipid peroxidation measurement in Cas9^{ctrl} and DHODH^{ko} NCI-H226 cells. Cells were grown in medium supplemented with uridine (50 μM). FIG. 4E is a graph showing cell survival fraction measurement in Cas9^{ctrl} and DHODH^{ko} NCI-H226 cells upon treatment with vehicle, uridine (50 μM), and uridine (50 μM)+Lip-1 (10 μM) for 24 hours. Error bars are mean±s.d, n=3 independent repeats. FIG. 4F is a graph showing cell viability measurement in Sh^{ctrl} and GPX4^{sh} HT-1080 cells treated with different doses of BQR for 4 hours. FIG. 4G is a graph showing lipid peroxidation measurement in Sh^{ctrl} and GPX4^{sh} HT-1080 cells upon treatment with BQR (500 μM) for 4 hours. Error bars are mean±s.d, n=3 independent repeats. FIG. 4H is a graph showing lipid peroxidation measurement in HT-1080 cells with indicated genotypes. Cells were grown in medium supplemented with uridine (50 μM). Error bars are mean±s.d, n=3 independent repeats. FIG. 4I is a graph showing cell survival fraction measurement in HT-1080 cells with indicated genotypes upon treatment with vehicle, uridine (50 μM), and uridine (50 μM)+Lip-1 (10 μM) for 24 hours. Error bars are mean±s.d, n=3 independent repeats. CTRP, Cancer Therapeutics Response Portal; BQR, brequinar; Lip-1, liproxstatin-1.

FIG. 5A, FIG. 5B, FIG. 5C, FIG. 5D, FIG. 5E, FIG. 5F, FIG. 5G, FIG. 5H, FIG. 5I, FIG. 5J, FIG. 5K, FIG. 5L, FIG. 5M, FIG. 5N, FIG. 5O, and FIG. 5P show DHODH deletion sensitizes GPX4^{high} cancer cells to ferroptosis. FIG. 5A is an image of DHODH protein levels in Cas9^{ctrl} and DHODH^{ko} GPX4^{high} cancer cell lines. Cells were grown in medium supplemented with uridine (50 μM). FIG. 5B is a graph showing DHO activity measurement in Cas9^{ctrl} and DHODH^{ko} HT-1080 cells. Cells were grown in medium supplemented with uridine (50 μM). Error bars are mean±s.d, n=3 independent repeats. FIG. 5C is a graph showing measurement of cell survival fraction in Cas9^{ctrl} and DHODH^{ko} HT-1080 cells upon treatment with vehicle or uridine (50 μM) for 24 hours. Error bars are mean±s.d, n=3 independent repeats. FIG. 5D is a graph showing PTGS2 mRNA measurement in Cas9^{ctrl} and DHODH^{ko} HT-1080 cells. Cells were grown in medium supplemented with uridine (50 μM). Error bars are mean±s.d, n=3 independent repeats. FIG. 5E is a graph showing lipid peroxidation measurement in Cas9^{ctrl} and DHODH^{ko} GPX4^{high} cell lines as indicated. Cells were grown in medium supplemented with uridine (50 μM). Error bars are mean±s.d, n=3 independent repeats. FIG. 5F is a graph showing cell viability measurement in Cas9^{ctrl} and DHODH^{ko} HT-1080 cells treated with different doses of ML162 for 4 hours. Cells were cultured in medium supplemented with uridine (50 μM). FIG. 5G is a graph showing measurement of cell survival fraction and PTGS2 mRNA levels in Cas9^{ctrl} and DHODH^{ko} HT-1080 cells upon treatment with RSL3 (1 μM) for 4 hours. Cells were grown in medium supplemented with uridine (50 μM). Error bars are mean±s.d, n=3 independent repeats. FIG. 5H is a Western blot analysis of DHODH and ACSL4 protein levels in HT-1080 cells with indicated genotypes. Cells were grown in medium supplemented with uridine (50 μM). FIG. 5I is a graph showing cell viability measurement in HT-1080 cells with indicated genotypes treated with different doses of RSL3 for 4 hours. Cells were grown in medium supplemented with uridine (50 μM). FIG. 5J is graphs showing measurement of SLC7A11, GPX4, or ACSL4 mRNA levels as well as their protein levels (by Western blot analysis) in Cas9^{ctrl} and DHODH^{ko} HT-1080 cells. Error bars are mean±s.d, n=3 independent repeats. FIG. 5K is a graph showing GSH levels measurement in Cas9^{ctrl} and DHODH^{ko} HT-1080 cells. Error bars are mean±s.d, n=3 independent repeats. FIG. 5L is an image showing DHODH protein levels in Cas9^{ctrl} and DHODH^{ko} GPX4^{low} cell lines. Cells were grown in medium supplemented with uridine (50 μM) and Lip-1 (10 μM). FIG. 5M is a graph showing DHO activity measurement in Cas9^{ctrl} and DHODH^{ko} NCI-H226 cells. Cells were grown in medium supplemented with uridine (50 μM) and Lip-1 (10 μM). Error bars are mean±s.d, n=3 independent repeats. FIG. 5N is a graph showing cell proliferation measurement of Cas9^{ctrl} and DHODH^{ko} NCI-H226 cells. Cells were grown in medium supplemented with uridine (50 μM). Error bars are mean±s.d, n=3 independent repeats. FIG. 5O is a graph showing Measurement of PTGS2 mRNA levels in Cas9^{ctrl} and DHODH^{ko} NCI-H226 cells. Cells were grown in medium supplemented with uridine (50 μM). Error bars are mean±s.d, n=3 independent repeats. FIG. 5P is a graph showing lipid peroxidation measurement of Cas9^{ctrl} and DHODH^{ko} GPX4^{low} cells. Cells were grown in medium supplemented with uridine (50 μM). Error bars are mean±s.d, n=3 independent repeats. DHO, dihydroorotate; GSH, glutathione.

FIG. 6A, FIG. 6B, FIG. 6C, FIG. 6D, FIG. 6E, FIG. 6F, FIG. 6G, FIG. 6H, FIG. 6I, FIG. 6J, FIG. 6K, FIG. 6L, FIG. 6M, FIG. 6N, FIG. 6O, FIG. 6P, and FIG. 6Q showing DHODH deletion induces ferroptosis in GPX4^{low} cancer cells. FIG. 6A is an image showing Western blot analysis of GPX4 and DHODH protein levels in Sh^{ctrl} and GPX4^{sh} HT-1080 cells. FIG. 6B is a graph showing cell proliferation measurement of Sh^{ctrl} and GPX4^{sh} HT-1080 cells. Error bars are mean±s.d, n=3 independent repeats. FIG. 6C is a graph showing cell viability measurement of Sh^{ctrl} and GPX4^{sh} HT-1080 cells treated with different doses of LFM or TF for 4 hours. FIG. 6D is graphs showing measurement of cell survival fraction and PTGS2 mRNA levels in Sh^{ctrl} and GPX4^{sh} HT-1080 cells upon treatment with BQR (500 μM) for 4 hours. Error bars are mean±s.d, n=3 independent repeats. FIG. 6E is an image showing Western blot analysis of GPX4 and DHODH protein levels in HT-1080 cells with indicated genotypes. Cells were grown in medium supplemented with uridine (50 μM). FIG. 6F is a graph showing measurement of PTGS2 mRNA levels in HT-1080 cells with indicated genotypes. Cells were grown in medium supplemented with uridine (50 μM). Error bars are mean±s.d, n=3 independent repeats. FIG. 6G is graphs showing cell proliferation measurement of DHODH^{ko} in Sh^{ctrl} or GPX4^{sh} HT-1080 cells. Cells were grown in medium supplemented with uridine (50 μM). Error bars are mean±s.d, n=3 independent repeats. FIG. 6H is an image showing Western blot analysis of DHODH and FSP1 protein levels in HT-1080 cells with indicated genotypes. Cells were grown in medium supplemented with uridine (50 μM). FIG. 6I is graphs showing cell viability measurement in Cas9^{ctrl} or DHODH^{ko} HT-1080 cells with indicated genotypes treated with different doses of RSL3 for 4 hours, following pretreatment with vehicle or Lip-1 (10 μM) for 24 hours. Cells were grown in medium supplemented with uridine (50 μM). FIG. 6J is an image showing Western blot analysis of DHODH and FSP1 protein levels in HT-1080 cells with indicated genotypes. Cells were grown in medium supplemented with uridine (50 μM). FIG. 6K is graphs showing cell viability measurement in Cas9^{ctrl} or DHODH^{ko} HT-1080 cells with indicated genotypes treated with different doses of RSL3 for 4 hours, following pretreatment with vehicle or Lip-1 (10 μM) for 24 hours. Cells were grown in medium supplemented with uridine (50 μM). FIG. 6L is a graph showing cell viability measurement in Cas9^{ctrl} or FSP1^{ko} HT-1080 cells treated with vehicle or BQR (500 μM), and different doses of RSL3 for 4 hours. FIG. 6M is a simplified schematic of DHODH and its mutants as indicated. FIG. 6N is an image showing Western blotting analysis of DHODH protein levels in cytosolic and mitochondrial fractions from DHODH^{ko} HT-1080 cells that express the indicated DHODH constructs. Cells were grown in medium supplemented with uridine (50 μM). FIG. 6O is a graph showing DHO activity measurement in DHODH^{ko} HT-1080 cells that express the indicated DHODH constructs. Cells were grown in medium supplemented with uridine (50 μM). Error bars are mean±s.d, n=3 independent repeats. FIG. 6P is a graph showing cell viability measurement in DHODH^{ko} HT-1080 cells that express the indicated DHODH constructs treated with different doses of ML162 for 4 hours. Cells were grown in medium supplemented with uridine (50 μM). FIG. 6Q is graphs showing measurement of cell survival fraction and PTGS2 mRNA levels in DHODH^{ko} HT-1080 cells that express the indicated DHODH constructs upon treatment with RSL3 (1 μM) for 4 hours. Cells were grown in medium supplemented with uridine (50 μM). Error bars are mean±s.d, n=3 independent repeats. LFM, leflunomide; TF, teriflunomide; BQR, brequinar; Lip-1, liproxstatin-1; MTS, mitochondrial targeting sequence; DHOD domain, dihydroorotate dehydrogenase domain; Cyto, cytosolic; Mito, mitochondrial.

FIG. 7A, FIG. 7B, FIG. 7C, FIG. 7D, FIG. 7E, FIG. 7F, FIG. 7G, FIG. 7H, FIG. 7I, FIG. 7J, and FIG. 7K show DHODH suppresses lipid peroxidation in mitochondria. FIG. 7A is a graph showing cell viability measurement in DHODH^{ko} HT-1080 cells that express the indicated DHODH constructs treated with different doses of RSL3 for 4 hours. Cells were grown in medium supplemented with uridine (50 μM). FIG. 7B is a graph showing lipid peroxidation measurement in DHODH^{ko} HT-1080 cells that express the indicated DHODH constructs upon treatment with RSL3 (1 μM) for 4 hours. Cells were grown in medium supplemented with uridine (50 μM). Error bars are mean±s.d, n=3 independent repeats. FIG. 7C is a graph showing cell viability measurement in GPX4^{sh} HT-1080 cells that express the indicated GPX4 constructs treated with different doses of BQR for 4 hours. FIG. 7D is a graph showing lipid peroxidation measurement in GPX4^{sh} HT-1080 cells that express the indicated GPX4 constructs upon treatment with BQR (500 μM) for 4 hours. Error bars are mean±s.d, n=3 independent repeats. FIG. 7E is a graph showing cell viability measurement in NCI-H226 cells that express the indicated GPX4 constructs treated with different doses of BQR for 4 hours. FIG. 7F is a graph showing lipid peroxidation measurement in NCI-H226 cells that express the indicated GPX4 constructs upon treatment with BQR (500 μM) for 4 hours. Error bars are mean±s.d, n=3 independent repeats. FIG. 7G and FIG. 7H are graphs showing cell viability measurement in Cas9^{ctrl} (FIG. 7G) or DHODH^{ko} (FIG. 7H) HT-1080 cells treated with different doses of RSL3 for 4 hours, following pretreatment with vehicle, TEMPO (10 μM), MitoTEMPO (10 μM), or Lip-1 (10 μM) for 24 hours. Cells were grown in medium supplemented with uridine (50 μM). FIG. 7I and FIG. 7J are graphs showing mitochondrial lipid peroxidation measurement in Cas9^{ctrl} (FIG. 7I) or DHODH^{ko} (FIG. 7J) HT-1080 cells upon treatment with RSL3 (1 μM) for 4 hours following pretreatment with vehicle, TEMPO (10 μM), MitoTEMPO (10 μM), or Lip-1 (10 μM) for 24 hours. Cells were grown in medium supplemented with uridine (50 μM). Error bars are mean±s.d, n=3 independent repeats. FIG. 7K is an image showing Cas9^{ctrl} or DHODH^{ko} HT-1080 cells were treated with RSL3 (1 μM) for 2 hours, then stained with mito-BODIPY. Oxidized mito-BODIPY (Green) indicates mitochondrial lipid peroxidation. Cells were grown in medium supplemented with uridine (50 μM). BQR, brequinar; Cyto, cytosolic; Mito, mitochondrial; TEMPO, 2,2,6,6-tetramethyl-1-piperidinyloxy; MitoTEMPO, 2-(2,2,6,6-tetramethylpiperidin-1-oxyl-4-ylamino)-2-oxoethyl) triphenylphosphonium chloride; Lip-1, liproxstatin-1; Mito-C11, fluorescent mitochondria-targeted lipid peroxidation probe.

FIG. 8A, FIG. 8B, FIG. 8C, FIG. 8D, FIG. 8E, FIG. 8F, and FIG. 8G show DHODH cooperates with mitochondrial GPX4 to suppresses ferroptosis in a variety of cell lines. FIG. 8A is an image showing Western blotting analysis of GPX4 levels in cytosolic and mitochondrial fractions in a panel of cancer cell lines. FIG. 8B is a simplified schematic of cytosolic and mitochondrial GPX4 constructs. FIG. 8C is an image showing Western blotting analysis of GPX4 protein levels in cytosolic and mitochondrial fractions from GPX4^{sh} HT-1080 cells that express the indicated GPX4 constructs. FIG. 8D is a graph showing cell viability measurement in GPX4^{sh} HT-1080 cells that express the indicated GPX4 constructs treated with different doses of LFM or TF for 4 hours. FIG. 8E is graphs showing measurement of cell survival fraction and PTGS2 mRNA levels in GPX4^{sh} HT-1080 cells that express the indicated GPX4 constructs upon treatment with BQR (500 μM) for 4 hours. Error bars are mean±s.d, n=3 independent repeats. FIG. 8F is an image showing Western blotting analysis of GPX4 protein levels in GPX4^{sh} cells that express the indicated GPX4 constructs in a variety of cell lines. FIG. 8G is graphs showing cell viability measurement in various GPX4^{sh} cells that express the indicated GPX4 constructs treated with different doses of BQR for 4 hours. Cyto, cytosolic; Mito, mitochondrial; MTS, mitochondrial targeting sequence; GSH peroxidase, glutathione peroxidase; LFM, leflunomide; TF, teriflunomide; BQR, brequinar.

FIG. 9A, FIG. 9B, FIG. 9C, FIG. 9D, FIG. 9E, FIG. 9F, FIG. 9G, FIG. 9H, FIG. 9I, FIG. 9J, FIG. 9K, FIG. 9L, FIG. 9M, FIG. 9N, FIG. 9O, FIG. 9P, FIG. 9Q, and FIG. 9R show inactivation of DHODH and GPX4 promotes mitochondrial lipid peroxidation. FIG. 9A is an image showing Western blot analysis of GPX4 protein levels in cytosolic and mitochondrial fractions from NCI-H226 cells that express the indicated GPX4 constructs. FIG. 9B is a graph showing cell proliferation measurement of NCI-H226 cells that express the indicated GPX4 constructs. Error bars are mean±s.d, n=3 independent repeats. FIG. 9C is graphs showing cell viability measurement in NCI-H226 cells that express the indicated GPX4 constructs treated with different doses of LFM or TF for 4 hours. FIG. 9D is graphs showing measurement of cell survival fraction and PTGS2 mRNA levels in NCI-H226 cells that express the indicated GPX4 constructs upon treatment with BQR (500 μM) for 4 hours. Error bars are mean±s.d, n=3 independent repeats. FIG. 9E is graphs showing cell viability measurement in Cas9^{ctrl} or DHODH^{ko} HT-1080 cells treated with different doses of ML162 for 4 hours, following pretreatment with vehicle, TEMPO (10 μM), MitoTEMPO (10 μM), or Lip-1 (10 μM) for 24 hours. Cells were grown in medium supplemented with uridine (50 μM). FIG. 9F is a graph showing mitochondrial lipid peroxidation measurement in Cas9^{ctrl} or DHODH^{ko} HT-1080 cells upon treatment with RSL3 (1 μM) for 4 hours. Cells were grown in medium supplemented with uridine (50 μM). Error bars are mean±s.d, n=3 independent repeats. FIG. 9G is a graph showing mitochondrial lipid peroxidation measurement in SW′ 1 or GPX4^{sh} HT-1080 cells upon treatment with BQR (500 μM) for 4 hours. Error bars are mean±s.d, n=3 independent repeats. FIG. 9H is graphs showing mitochondrial lipid peroxidation measurement of HT-1080 cells upon treatment with RSL3 (1 μM) and/or BQR (500 μM), LFM (100 μM), or TF (500 μM) for 4 hours, following pretreatment with vehicle or Lip-1 (10 μM) for 24 hours. Error bars are mean±s.d, n=3 independent repeats. FIG. 9I is graphs showing mitochondrial lipid peroxidation measurement of HT-1080 cells upon treatment with ML162 (1 μM) and/or BQR (500 μM), LFM (100 μM), or TF (500 μM) for 4 hours, following pretreatment with vehicle or Lip-1 (10 μM) for 24 hours. Error bars are mean±s.d, n=3 independent repeats. FIG. 9J is a graph showing mitochondrial lipid peroxidation measurement of DHODH^{ko} HT-1080 cells that express the indicated DHODH constructs upon treatment with RSL3 (1 μM) for 4 hours. Cells were grown in medium supplemented with uridine (50 μM). Error bars are mean±s.d, n=3 independent repeats. FIG. 9K and FIG. 9L are graphs showing mitochondrial lipid peroxidation measurement in Cas9^{ctrl} or DHODH^{ko} HT-1080 cells with indicated genotypes upon treatment with RSL3 (1 μM) for 4 hours, following pretreatment with vehicle or Lip-1 (10 μM) for 24 hours. Cells were grown in medium supplemented with uridine (50 μM). Error bars are mean±s.d, n=3 independent repeats. FIG. 9M is a graph showing mitochondrial lipid peroxidation measurement in Cas9^{ctrl} or FSP1^{ko} HT-1080 cells upon treatment with RSL3 (1 μM) and/or BQR (500 μM) for 4 hours. Error bars are mean±s.d, n=3 independent repeats. FIG. 9N is an image showing Western blot analysis of DHODH and FSP1 protein levels in cytosolic (cyto) and mitochondrial (mito) fractions of HT-1080 cells with indicated genotypes. Cells were grown in medium supplemented with uridine (50 μM). FIG. 9O is graphs showing cell viability measurement in Cas9^{ctrl} or DHODH^{ko} HT-1080 cells with indicated genotypes treated with different doses of RSL3 for 4 hours, following pretreatment with vehicle or Lip-1 (10 μM) for 24 hours. Cells were grown in medium supplemented with uridine (50 μM). FIG. 9P is graphs showing mitochondrial lipid peroxidation measurement in Cas9^{ctrl} or DHODH^{ko} HT-1080 cells with indicated genotypes upon treatment with RSL3 (1 μM) for 4 hours, following pretreatment with vehicle or Lip-1 (10 μM) for 24 hours. Cells were grown in medium supplemented with uridine (50 μM). Error bars are mean±s.d, n=3 independent repeats. FIG. 9Q is a graph showing mitochondrial lipid peroxidation measurement of GPX4^{sh} HT-1080 cells that express the indicated GPX4 constructs upon treatment with BQR (500 μM) for 4 hours. Error bars are mean±s.d, n=3 independent repeats. FIG. 9R is a graph showing mitochondrial lipid peroxidation measurement of NCI-H226 cells that express the indicated GPX4 constructs upon treatment with BQR (500 μM) for 4 hours. Error bars are mean±s.d, n=3 independent repeats. Cyto, cytosolic; Mito, mitochondrial; LFM, leflunomide; TF, teriflunomide; BQR, brequinar; TEMPO, 2,2,6,6-tetramethyl-1-piperidinyloxy; MitoTEMPO, 2-(2,2,6,6-tetramethylpiperidin-1-oxyl-4-ylamino)-2-oxoethyl) triphenylphosphonium chloride; Lip-1, liproxstatin-1.

FIG. 10A, FIG. 10B, FIG. 10C, FIG. 10D, FIG. 10E, FIG. 10F, FIG. 10G, FIG. 10H, FIG. 10I, FIG. 10J, FIG. 10K, FIG. 10L, FIG. 10M, and FIG. 10N show DHODH suppresses ferroptosis likely through reducing CoQ to CoQH₂. FIG. 10A is a graph showing cell viability measurement in HT-1080 cells treated with different doses of FIN56 and co-treatment with vehicle or BQR (500 μM) for 4 hours, following pretreatment with vehicle or Lip-1 (10 μM) for 24 hours. FIG. 10B is a graph showing mitochondrial lipid peroxidation measurement in HT-1080 cells upon treatment with vehicle, FIN56 (50 μM), and/or BQR (500 μM) for 4 hours, following pretreatment with vehicle or Lip-1 (10 μM) for 24 hours. Error bars are mean±s.d, n=3 independent repeats. FIG. 10C and FIG. 10D are graphs showing cell viability measurement in Cas9^{ctrl} (FIG. 10C) or DHODH^{ko} (FIG. 10D) HT-1080 cells with indicated genotypes treated with different doses of RSL3 for 4 hours, following pretreatment with vehicle or Lip-1 (10 μM) for 24 hours. Cells were grown in medium supplemented with uridine (50 μM). FIG. 10E and FIG. 10F are graphs showing cell viability measurement in Cas9^{ctrl} (FIG. 10E) or DHODH^{ko} (FIG. 10F) HT-1080 cells treated with different doses of RSL3 for 4 hours, following pretreatment with vehicle, 4-CBA (5 mM), or 4-CBA (5 mM)+Lip-1 (10 μM) for 24 hours. Cells were grown in medium supplemented with uridine (50 μM). FIG. 10G and FIG. 10H are graphs showing CoQ/CoQH₂ ratio measurement in HT-1080 (FIG. 10G) or NCI-H226 (FIG. 10H) cells that were treated with BQR (1 mM) for 2 hours. Error bars are mean±s.d, n=3 independent repeats. FIG. 10I and FIG. 10J are graphs showing cell viability measurement in Cas9^{ctrl} (FIG. 10I) or DHODH^{ko} (FIG. 10J) HT-1080 cells treated with different doses of RSL3 for 4 hours, following pretreatment with vehicle, MitoQ (10 μM), MitoQH₂ (10 μM), or Lip-1 (10 μM) for 24 hours. Cells were grown in medium supplemented with uridine (50 μM). FIG. 10K and FIG. 10L are graphs showing mitochondrial lipid peroxidation measurement of Cas9^{ctrl} (FIG. 10K) or DHODH^{ko} (FIG. 10L) HT-1080 cells upon treatment with RSL3 (1 μM) for 4 hours, following pretreatment with vehicle, MitoQ (10 μM), MitoQH₂ (10 μM), or Lip-1 (10 μM) for 24 hours. Cells were grown in medium supplemented with uridine (50 μM). Error bars are mean±s.d, n=3 independent repeats. FIG. 10M is a graph showing cell viability measurement in HT-1080 cells with indicated genotypes treated with different doses of RSL3 for 4 hours. Cells were grown in medium supplemented with uridine (50 μM). FIG. 10N is a graph showing mitochondrial lipid peroxidation measurement of HT-1080 cells with indicated genotypes upon treatment with RSL3 (1 μM) for 4 hours. Cells were grown in medium supplemented with uridine (50 μM). Error bars are mean±s.d, n=3 independent repeats. BQR, brequinar; Lip-1, liproxstatin-1; 4-CBA, 4-Carboxybenzaldehyde; DHO, dihydroorotate; OA, orotate; FMN, flavin mononucleotide; FMNH₂, reduced flavin mononucleotide; CoQ, coenzyme Q; CoQH₂, reduced coenzyme Q. MitoQ, [10-(4,5-dimethoxy-2-methyl-3,6-dioxo-1,4-cyclohexadien-1-yl)decyl] triphenyl-phosphonium, monomethanesulfonate; MitoQH₂, [10-(2,5-dihydroxy-3,4-dimethoxy-6-methylphenyl)decyl] triphenyl-phosphonium, monomethanesulfonate.

FIG. 11A, FIG. 11B, FIG. 11C, FIG. 11D, FIG. 11E, FIG. 11F, FIG. 11G, FIG. 11H, FIG. 11I, FIG. 11J, and FIG. 11K show DHODH regulation of ferroptosis relates to its function to reduce CoQ to CoQH₂ in mitochondria. FIG. 11A is graphs showing cell viability measurement in HT-1080 cells treated with different doses of FIN56 and co-treatment with LFM (100 μM) or TF (500 μM) for 4 hours, following pretreatment with vehicle or Lip-1 (10 μM) for 24 hours. FIG. 11B is graphs showing measurement of cell survival fraction and PTGS2 mRNA levels in HT-1080 cells upon treatment with vehicle, FIN56 (50 μM) and/or BQR (500 μM) for 4 hours, following pretreatment with vehicle or Lip-1 (10 μM) for 24 hours. Error bars are mean±s.d, n=3 independent repeats. FIG. 11C is an image showing Western blot analysis of COQ2 and DHODH protein levels in HT-1080 cells with indicated genotypes. Cells were grown in medium supplemented with uridine (50 μM). FIG. 11D is a graph showing total CoQ measurement in Cas9^{ctrl} or COQ2^{ko} HT-1080 cells. Error bars are mean±s.d, n=3 independent repeats. FIG. 11E is a graph showing total CoQ measurement in HT-1080 cells that were treated with vehicle or 4-CBA (5 mM) for 24 hours. Error bars are mean±s.d, n=3 independent repeats. FIG. 11F is graphs showing cell viability measurement in Cas9^{ctrl} and DHODH^{ko} HT-1080 cells with indicated genotypes treated with different doses of ML162 for 4 hours, following pretreatment with vehicle or Lip-1 (10 μM) for 24 hours. Cells were grown in medium supplemented with uridine (50 μM). FIG. 11G is graphs showing cell viability measurement in Cas9^{ctrl} and DHODH^{ko} HT-1080 cells treated with different doses of ML162 for 4 hours, following pretreatment with vehicle, 4-CBA (5 mM) and Lip-1 (10 μM) for 24 hours. Cells were grown in medium supplemented with uridine (50 μM). FIG. 11H is graphs showing mitochondrial lipid peroxidation measurement in Cas9^{ctrl} or DHODH^{ko} HT-1080 cells upon treatment with RSL3 (1 μM), following pretreatment with vehicle, 4-CBA (5 mM), or 4-CBA (5 mM)+Lip-1 (10 μM) for 24 hours. Cells were grown in medium supplemented with uridine (50 μM). Error bars are mean±s.d, n=3 independent repeats. FIG. 11I is a schematic showing how DHODH couples the oxidation of DHO to OA to the reduction of CoQ to CoQH₂ in the mitochondrial inner membrane. FIG. 11J is graphs showing cell viability measurement in Cas9^{ctrl} and DHODH^{ko} HT-1080 cells treated with different doses of ML162 for 4 hours, following pretreatment with vehicle, MitoQ (10 μM), MitoQH₂ (10 μM), or Lip-1 (10 μM) for 24 hours. Cells were grown in medium supplemented with uridine (50 μM). FIG. 11K is graphs showing lipid peroxidation measurement of Cas9^{ctrl} and DHODH^{ko} HT-1080 cells upon treatment with RSL3 (1 μM) for 4 hours, following pretreatment with vehicle, MitoQ (10 μM), MitoQH₂ (10 μM), or Lip-1 (10 μM) for 24 hours. Cells were grown in medium supplemented with uridine (50 μM). Error bars are mean±s.d, n=3 independent repeats. BQR, brequinar; LFM, leflunomide; TF, teriflunomide; 4-CBA, 4-Carboxybenzaldehyde; DHO, dihydroorotate; OA, orotate; FMN, oxidized flavin mononucleotide; FMNH₂, reduced flavin mononucleotide; CoQH₂, reduced coenzyme Q; CoQ, oxidized coenzyme Q; OCR, oxygen consumption rate; MitoQ, [10-(4,5-dimethoxy-2-methyl-3,6-dioxo-1,4-cyclohexadien-1-yl)decyl] triphenyl-phosphonium, monomethanesulfonate; MitoQH₂, [10-(2,5-dihydroxy-3,4-dimethoxy-6-methylphenyl)decyl] triphenyl-phosphonium, monomethanesulfonate; Lip-1, liproxstatin-1.

FIG. 12A, FIG. 12B, FIG. 12C, FIG. 12D, FIG. 12E, FIG. 12F, FIG. 12G, FIG. 12H, FIG. 12I, FIG. 12J, FIG. 12K, FIG. 12L, FIG. 12M, FIG. 12N, and FIG. 12O show the effect of MitoQ and MitoQH₂ on RSL3 and BQR-induced ferroptosis in a variety of cell lines. FIG. 12A is an image showing Western blot analysis of GPX4, DHODH and FSP1 protein levels in indicated cell lines. FIG. 12B, FIG. 12C, FIG. 12D, FIG. 12E, FIG. 12F, FIG. 12G, FIG. 12H, FIG. 12I, and FIG. 12J are grpahs showing cell viability measurement in 293T (FIG. 12B), Hela (FIG. 12C), Jurkat (FIG. 12D), SW620 (FIG. 12E), U-87 MG (FIG. 12F), A549 (FIG. 12G), NCI-H1437 (FIG. 12H), MDA-MB-436 (FIG. 12I), and MDA-MB-231 (FIG. 12J) cells treated with different doses of RSL3 with vehicle (top panel) or BQR (500 μM) (bottom panel) for 4 hours, following pretreatment with vehicle, MitoQ (10 μM), MitoQH₂ (10 μM), or Lip-1 (10 μM) for 24 hours. FIG. 12K is a graph showing CoQ/CoQH₂ ratio measurement in HT-1080 cells that were treated with myxothiazol (10 μM) for 2 hours. Error bars are mean±s.d, n=3 independent repeats. FIG. 12L is graphs showing cell viability measurement in Cas9^{ctrl} and DHODH^{ko} HT-1080 cells treated with different doses of RSL3 for 4 hours, following pretreatment with vehicle or myxothiazol (1 μM) for 24 hours. Cells were grown in medium supplemented with uridine (50 μM). FIG. 12M is a graph showing CoQ/CoQH₂ ratio measurement in A549 cells that were treated with myxothiazol (10 μM) for 2 hours. Error bars are mean±s.d, n=3 independent repeats. FIG. 12N is graphs showing cell viability measurement in A549 cells treated with different doses of RSL3 with or without BQR (500 μM) for 4 hours, following pretreatment with vehicle or myxothiazol (1 μM) for 24 hours. FIG. 12O is an image showing Western blot analysis of DHODH and ciAOX protein levels in HT-1080 cells with indicated genotypes. Cells were grown in medium supplemented with uridine (50 μM). MitoQ, [10-(4,5-dimethoxy-2-methyl-3,6-dioxo-1,4-cyclohexadien-1-yl)decyl] triphenyl-phosphonium, monomethanesulfonate; MitoQH₂, [10-(2,5-dihydroxy-3,4-dimethoxy-6-methylphenyl)decyl] triphenyl-phosphonium, monomethanesulfonate; Lip-1, liproxstatin-1; OCR, oxygen consumption rate.

FIG. 13A, FIG. 13B, FIG. 13C, FIG. 13D, FIG. 13E, FIG. 13F, FIG. 13G, FIG. 13H, FIG. 13I, FIG. 13J, FIG. 13K, and FIG. 13L show DHODH inhibitor treatment suppresses GPX4^{low} tumor growth through inducing ferroptosis in vivo. FIG. 13A and FIG. 13B are graphs showing volumes of Sh^{ctrl} (FIG. 13A) and GPX4^{sh} (FIG. 13B) HT-1080 xenograft tumors with the indicated treatments at different time points (days). Error bars are mean±s.d, n=8 independent tumors. FIG. 13C and FIG. 13D are graphs showing immunochemistry scoring of 4-HNE staining of Sh^{ctrl} (FIG. 13C) and GPX4^{sh} (FIG. 13D) HT-1080 xenograft tumors. Error bars are mean±s.d, n=5 independent tumors. FIG. 13E is a graph showing volumes of NCI-H226 cells xenograft tumors with the indicated treatments at different time points (days). Error bars are mean±s.d, n=8 independent tumors. FIG. 13F is an image showing Western blot analysis of GPX4 protein levels in different PDX models. FIG. 13G, FIG. 13H, and FIG. 13I are graphs showing volumes of TC632 (FIG. 13G), TC629 (FIG. 13H), or TC494 (FIG. 13I) PDX tumors with the indicated treatments at different time points (days). Error bars are mean±s.d, n=6 independent tumors. FIG. 13J is a graph showing volumes of HT-1080 xenograft tumors with the indicated treatments at different time points (days). Error bars are mean±s.d, n=8 independent tumors. FIG. 13K is a graph showing immunochemistry scoring of 4-HNE staining of HT-1080 xenograft tumors. Error bars are mean±s.d, n=5 independent tumors. FIG. 13L is a graph showing volumes of TC632 PDXs tumors with the indicated treatments at different time points (days). Error bars are mean±s.d, n=6 independent tumors. BQR, brequinar; Lip-1, liproxstatin-1; 4-HNE, 4-Hydroxynonenal; PDX, patient-derived xenograft; SAS, sulfasalazine.

FIG. 14A, FIG. 14B, FIG. 14C, FIG. 14D, FIG. 14E, FIG. 14F, FIG. 14G, FIG. 14H, FIG. 14I, FIG. 14J, FIG. 14K, FIG. 14L, FIG. 14M, FIG. 14N, and FIG. 14O show DHODH inhibitor selectively suppresses GPX4^{low} tumor growth. FIG. 14A is graphs showing weight measurement of Sh^{ctrl} and GPX4^{sh} HT-1080 xenograft tumors with the indicated treatments. Error bars are mean±s.d, n=8 independent tumors. FIG. 14B, FIG. 14C, and FIG. 14D are representative immunochemical images from Sh^{ctrl} and GPX4^{sh} HT-1080 xenograft tumors with the indicated treatments (FIG. 14B); staining scores of cleaved-caspase 3 (FIG. 14C) and ki67 (FIG. 14D) staining are also shown. Error bars are mean±s.d, n=5 independent tumors. FIG. 14E is a graph showing weight measurement of NCI-H226 xenograft tumors with the indicated treatments. Error bars are mean±s.d, n=8 independent tumors. FIG. 14F is graphs showing weight measurement of TC632, TC629, or TC494 PDXs tumors with the indicated treatments. Error bars are mean±s.d, n=6 independent tumors. FIG. 14G is graphs showing volumes of Cas9^{ctrl} and DHODH^{ko} NCI-H226 xenograft tumors with the indicated treatments at different time points (days). Error bars are mean±s.d, n=8 independent tumors. FIG. 14H is graphs showing weight measurement of Cas9^{ctrl} and DHODH^{ko} NCI-H226 xenograft tumors with the indicated treatments. Error bars are mean±s.d, n=8 independent tumors. FIG. 14I is a graph showing weight measurement of HT-1080 xenograft tumors with the indicated treatments. Error bars are mean±s.d, n=8 independent repeats. FIG. 14J, FIG. 14K, and FIG. 14L are representative immunochemistry images of HT-1080 xenograft tumors with the indicated treatments (FIG. 14J); staining scores of cleaved-caspase 3 (FIG. 14K) and ki67 (FIG. 14L) staining are also shown. Error bars are mean±s.d, n=5 independent tumors. FIG. 14M is a graph showing volumes of TC629 PDXs tumors with the indicated treatments at different time points (days). Error bars are mean±s.d, n=6 independent tumors. FIG. 14N is graphs showing weight measurement of TC632 and TC629 PDX tumors with the indicated treatments. Error bars are mean±s.d, n=6 independent tumors. FIG. 14O are graphs showing mice weight measurement of all cell line xenografts or PDXs with different treatments at different time points (days) as indicated. Error bars are mean±s.d, n=8 (nude mice) or n=6 (NSG mice) independent mice. BQR, brequinar; Lip-1, liproxstatin-1; H&E, hematoxylin and eosin; 4-HNE, 4-Hydroxynonenal; SAS, sulfasalazine; PDX, patient-derived xenograft.

FIG. 15 is a diagram depicting ferroptosis suppression at different subcellular compartments. PLOOH, phospholipid hydroperoxide; PLOO·, phospholipid hydroperoxyl radical; GSH, reduced glutathione; GSSH, oxidized glutathione; NAD(P)H, reduced nicotinamide adenine dinucleotide (phosphate); NAD(P)⁺, oxidized nicotinamide adenine dinucleotide (phosphate); CoQ, oxidized coenzyme Q; CoQH₂, reduced coenzyme Q; FMNH₂, reduced flavin mononucleotide; FMN, oxidized flavin mononucleotide; Asp, aspartate; C-P, carbamoyl phosphate; P, phosphate; C-Asp, N-Carbamoyl-L-aspartate; DHO, dihydroorotate; OA, orotate; PRPP, phosphoribosyl pyrophosphate; PPi, inorganic pyrophosphate; OMP, orotidine 5′-monophosphate; CO₂, carbon dioxide; UMP, uridine 5′-monophosphate.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a,” “an,” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related.

The term “about,” as used herein, refers to a value or composition that is within an acceptable error range for the particular value or composition as determined by one of ordinary skill in the art, which will depend in part on how the value or composition is measured or determined, i.e., the limitations of the measurement system. For example, about can include the recited number±10% (for example, “about 10” means 9 to 11).

The term “administering,” as used herein, refers to the physical introduction of a composition comprising a therapeutic agent (e.g., a dihydroorotate dehydrogenase (DHODH) inhibitor and/or a ferroptosis inducer) to a subject, using any of the various methods and delivery systems known to those skilled in the art. Routes of administration include oral, intravenous, intramuscular, subcutaneous, intraperitoneal, spinal or other parenteral routes of administration, for example by injection or infusion.

The term an “anti-cancer agent” or combination thereof promotes cancer regression in a subject. In some aspects, a therapeutically effective amount of the therapeutic agent promotes cancer regression to the point of eliminating the cancer.

The term “immunotherapy” refers to the treatment of a subject afflicted with, or at risk of contracting or suffering a recurrence of, a disease by a method comprising inducing, enhancing, suppressing or otherwise modifying an immune response.

The terms “sample” or “biological sample” as used herein refers to biological material isolated from a subject. The sample can contain any biological material suitable for determining gene expression, for example, by sequencing nucleic acids.

The sample can be any suitable biological tissue, for example, cancer tissue. In one aspect, the sample is a tumor tissue biopsy, e.g., a formalin-fixed, paraffin embedded (FFPE) tumor tissue or a fresh-frozen tumor tissue or the like. In another aspect, an intratumoral sample is used. In another aspect, biological fluids can be present in a tumor tissue biopsy, but the biological sample will not be a biological fluid per se.

The term a “cancer,” as used herein, refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. The term “tumor” refers to a solid cancer. The term “carcinoma” refers to a cancer of epithelial origin.

The term “control sample,” as used herein refers to a biological sample (e.g. blood, urine, tumor) obtained from a “normal” or “healthy” individual(s) that is believed not to have cancer or from a “normal” or “healthy” (e.g., non-cancerous) biological sample from an individual(s) that is believed to have cancer. Controls may be selected using methods that are well known in the art. Once a level has become well established for a control population, array results from test biological samples can be directly compared with the known levels.

The terms “glutathione peroxidase 4” or “GPX4,” as used herein refers to an enzyme that encodes the GPX4 gene. GPX4 is a phospholipid hydroperoxidase that protects cells against membrane lipid peroxidation.

As used herein, the term “subject” includes any human or nonhuman animal. The terms, “subject” and “patient” are used interchangeably herein. The term “nonhuman animal” includes, but is not limited to, vertebrates such as dogs, cats, horses, cows, pigs, boar, sheep, goat, buffalo, bison, llama, deer, elk and other large animals, as well as their young, including calves and lambs, and to mice, rats, rabbits, guinea pigs, primates such as monkeys and other experimental animals. Within animals, mammals are preferred, most preferably, valued and valuable animals such as domestic pets, race horses and animals used to directly produce (e.g., meat) or indirectly produce (e.g., milk) food for human consumption, although experimental animals are also included. In specific aspects, the subject is a human. Thus, the present disclosure is applicable to clinical, veterinary and research uses.

The terms “treat,” “treating,” “treatment,” and the like as used herein refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of DHODH inhibitor and/or the ferroptosis inducer to an individual in need of such treatment.

The term “therapeutically effective amount” or “effective dose” as used herein refers to an amount of the active ingredient(s) that is (are) sufficient, when administered by a method of the disclosure, to efficaciously deliver the active ingredient(s) for the treatment of condition or disease of interest to an individual in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; modulate protein methylation in the target cells; and/or relieve, to some extent, one or more of the symptoms associated with the cancer. To the extent the administered compound or composition prevents growth and/or kills existing cancer cells, it may be cytostatic and/or cytotoxic.

In addition, the terms “effective” and “effectiveness” with regard to a treatment disclosed herein includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

The ability of a therapeutic agent to promote disease regression, e.g., cancer regression, can be evaluated using a variety of methods known to the skilled practitioner, such as in human subjects during clinical trials, in animal model systems predictive of efficacy in humans, or by assaying the activity of the agent in in vitro assays.

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

It is understood that wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

### II. Therapeutic Methods

One aspect of the present disclosure is directed to a method for treating cancer, the method comprising administering to a subject in need thereof a therapeutically effective amount of a dihydroorotate dehydrogenase (DHODH) inhibitor, wherein the cancer has an altered glutathione peroxidase 4 (GPX4) expression as compared to a control sample.

In some aspects, the altered GPX4 expression is a low expression level of GPX4 as compared to a control sample. The “expression level” when used to refer to GPX4 (e.g., an altered GPX4 expression level, low GPX4 expression level, low GPX4 expression, high GPX4 expression level, or high GPX4 expression) includes the expression level of GPX4 RNA, GPX4 protein, or both. When referring to GPX4 RNA, expression level generally refers to a detected quantity of RNA molecules representing the nucleic acid sequence of interest present in a subject or sample therefrom or a control sample, e.g., the quantity of RNA molecules expressed from a DNA molecule (e.g., from the genome of the subject or the subject's cancer) comprising the nucleic acid sequence of interest. When referring to GPX4 protein, expression level generally refers to a detected quantity of protein molecules representing the protein of interest present in a subject or sample therefrom or a control sample, e.g., the quantity of protein molecules expressed from an RNA molecule (e.g., from the transcriptome of the subject or the subject's cancer) comprising the ribonucleic acid sequence of interest.

In some aspects, the cancer is a tumor. In a further aspect, the tumor is a carcinoma.

In some aspects, the tumor is a solid tumor. A “solid tumor” includes, but is not limited to, sarcoma, melanoma, carcinoma, or other solid tumor cancer.

The term “sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzkycell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be treated according to the methods disclosed herein include, e.g., leukemia, Hodgkin's disease, non-Hodgkin's lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

In some aspects, the cancer is relapsed, refractory, or refractory following at least one prior therapy comprising administration of at least one anti-cancer agent. The term “relapsed” refers to a situation where a subject, that has had a remission of cancer after a therapy, has a return of cancer cells. As used herein, the term “refractory” or “resistant” refers to a circumstance where a subject, even after intensive treatment, has residual cancer cells in the body. In some aspects, the cancer is metastatic.

In some aspects, the cancer can include, but not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In some aspects, the cancer is selected from fibrosarcoma, lung squamous cell carcinoma, lung adenocarcinoma, renal cell carcinoma, breast adenocarcinoma, colorectal adenocarcinoma, endocervical adenocarcinoma, or T acute lymphoblastic leukemia.

A “cancer” or “cancer tissue” can include a tumor at various stages. In certain aspects, the cancer or tumor is stage 0, such that, e.g., the cancer or tumor is very early in development and has not metastasized. In some aspects, the cancer or tumor is stage I, such that, e.g., the cancer or tumor is relatively small in size, has not spread into nearby tissue, and has not metastasized. In other aspects, the cancer or tumor is stage II or stage III, such that, e.g., the cancer or tumor is larger than in stage 0 or stage I, and it has grown into neighboring tissues but it has not metastasized, except potentially to the lymph nodes. In other aspects, the cancer or tumor is stage IV, such that, e.g., the cancer or tumor has metastasized. Stage IV can also be referred to as advanced or metastatic cancer. Dihydroorotate dehydrogenase (DHODH) inhibitors have been disclosed for the treatment or prevention of autoimmune diseases, immune and inflammatory diseases, destructive bone disorders, malignant neoplastic diseases, angiogenic-related disorders, viral diseases, and infectious diseases. See, for example, WO2010083975; WO2011138665; WO200137081; WO2009133379; WO 2009021696; WO200 082691; WO2009029473; WO2009153043; US2009209557; US2009 062318; US2009082374; WO2008097180; W02QQ8Q77639; US2008027079; US2007 299U4; US2007027193; US2007224672; WO2007149211; JP2007015952; WO2006 044741; WO2006001961; WO2006051937; WO2006038606; WO2006022442; US2006 199856; WO2005075410; U.S. Pat. Nos. 10,016,402; 7,074,831; WO2004056797; U.S. Pat. No. 7,247,736; WO2004056747; WO 2004056746; JP2004099586; WO2003097574; WO2003030905; WO2003006425; WO2003 006424; US2003203951; WO2002080897; U.S. Pat. Nos. 7,176,241; 7,423,057; WO2001024785; U.S. Pat. No. 6,841,561; WO9945926; WO9938846; WO9941239; EP767167 and U.S. Pat. No. 5,976,848, each of which are herein incorporated by reference in their entirety. For additional reviews and literature regarding DHODH inhibitors see Bio &Med. Chem. Letters, 20(6), 2010, pages 1981-1984; Med. Chem. 2009, 52, 2683-2693; J. Med. Chem. 2008, 51 (12), 3649-3653.

For example, dihydroorotate dehydrogenase (DHODH) inhibitors include, but are not limited to, leflunomide, teriflunomide, brequinar, Dichloroallyl lawsone, maritimus, redoxal, Ag-636, ASLAN003, BAY2402234, IMU-838, PP-001, and PTC299.

In some aspects, the dihydroorotate dehydrogenase (DHODH) inhibitor is selected from the group consisting of Ag-636, ASLAN003, BAY2402234, leflunomide, brequinar, teriflunomide, IMU-838, PP-001, PTC299, and combinations thereof. In a further aspect, the dihydroorotate dehydrogenase (DHODH) inhibitor is selected from the group consisting of leflunomide, brequinar, teriflunomide, and combinations thereof.

In some aspects, the altered GPX4 expression is a high expression level of GPX4 as compared to a control sample.

In some aspects, the method further comprises administering a therapeutically effective amount of a ferroptosis inducer.

Ferroptosis inducers (FIN) have been disclosed for the treatment or prevention of malignant neoplastic diseases, nervous system diseases, and blood diseases. See, for example, US20200138829, WO2019200343, WO2018218087, and WO2020236620 each of which are herein incorporated by reference in their entirety.

Ferroptosis inducers (FIN) are divided into four classes (i) inhibit system Xc− and prevent cysteine import; (ii) inhibit GPX4; (iii) degrade GPX4, bind to SQS and deplete antioxidant CoQ10; and (iv) oxidize ferrous iron and lipidome directly and invactive GPX4 directly. See, for example, Li et al. Cell Death & Disease, 11, 88 (2020), which is incorporated by reference in its entirety. Class I includes, for example, erastin, sorafenib, sulfasalazine; Class II includes, for example, RSL3, (1S,3R)-RSL3, ML210, ML162, DPI7, and DPI10; Class III includes, for example, FIN56; and claim 4 includes, for example FINO2.

In some aspects, the ferroptosis inducer is a class I ferrotosis inducer, a class II ferroptosis inducer, or a combination thereof.

In some aspects, the ferroptosis inducer is selected from the group consisting of sulfasalazine, rosiglitazone, rosiglitazone maleate, bardoxolone methyl, linagliptin, curcumin, zileuton, pioglitazone HCl, nordihydroguaiaretic acid (NDGA), troglitazone, setanaxib, deferoxamine mesylate, sorafenib tosylate, cisplatin, rosadustat, lapatinib, simvastatin, deferasirox, sorafenib, erastin, imidazole ketone erastin, and combinations thereof. In a further aspect, the ferroptosis inducer is selected from the group consisting of sulfasalazine RSL3, (1S,3R)-RSL3, ML210, ML162, and combinations thereof.

In some aspects, the ferroptosis inducer is not a class (iii) ferroptosis inducer. In some aspects, the DHODH inhibitor is administered in combination with a class (i) ferroptosis inducer. In some aspects, the DHODH inhibitor is administered in combination with a class (ii) ferroptosis inducer. In some aspects, the DHODH inhibitor is administered in combination with a class (iv) ferroptosis inducer.

In some aspects, the subject is a human.

Another aspect of the present disclosure is directed to a method of treating a subject with a dihydroorotate dehydrogenase (DHODH) inhibitor, wherein the subject is afflicted with a cancer, the method comprising (a) determining, in the cancer sample, the expression level of glutathione peroxidase 4 (GPX4) and (b) if the expression level of GPX4 is low as compared to relative GPX4 expression level in a control sample, then administering a therapeutically effective amount of the DHODH inhibitor to the subject, or (c) if the expression level of GPX4 is high as compared to relative GPX4 expression level in a control sample, then administering a therapeutically effective amount of the DHODH inhibitor and a therapeutically effective amount of a ferroptosis inducer to the subject.

Another aspect of the present disclosure is directed to a method of treating a subject afflicted with a cancer, comprising administering to the subject a therapeutically effective amount of a dihydroorotate dehydrogenase (DHODH) inhibitor, wherein, prior to the administration, the subject is identified as exhibiting an altered expression level of glutathione peroxidase 4 (GPX4) as compared to relative GPX4 expression level in a control sample.

In some aspects, if the subject is identified as exhibiting a low expression level of GPX4, the treatment comprises administering a therapeutically effective amount of the DHODH inhibitor to the subject.

In some aspects, if the subject is identified as exhibiting a high expression level of GPX4, the treatment comprises administering a therapeutically effective amount of the DHODH inhibitor and a therapeutically effective amount of a ferroptosis inducer to the subject.

In some aspects, identifying the subject as exhibiting an altered expression level of GPX4 comprises obtaining a cancer sample from the subject and analyzing the sample for the GPX4 expression level.

Another aspect of the present disclosure is directed to a method of identifying a subject afflicted with a cancer as suitable for treatment with a dihydroorotate dehydrogenase (DHODH) inhibitor, the method comprising determining whether the subject has an altered expression level of glutathione peroxidase 4 (GPX4) as compared to relative GPX4 expression level in a control sample, wherein (a) if the expression level of GPX4 is low as compared to relative GPX4 expression level in a control sample, then a therapeutically effective amount of the DHODH inhibitor can be administered to the subject, or (b) if the expression level of GPX4 is high as compared to relative GPX4 expression level in a control sample, then a therapeutically effective amount of the DHODH inhibitor and a therapeutically effective amount of a ferroptosis inducer can be administered to the subject.

In some aspects, determining whether the subject has an altered expression level of GPX4 comprises obtaining a cancer sample from the subject and analyzing the sample for the GPX4 expression level.

The level of GPX4 expression as described herein can be determined using any method in the art. For example, expression levels can be determined by detecting expression of nucleic acids (e.g., RNA or mRNA) or proteins encoded by the gene. Thus, in some aspects, the expression levels are transcribed RNA levels and/or expressed protein levels. In some aspects, the RNA levels are determined using sequencing methods, e.g., Next Generation Sequencing (NGS). In some aspects, the NGS is RNA-Seq, EdgeSeq, PCR, Nanostring, or combinations thereof, or any technologies that measure RNA. In some aspects, the RNA measurement methods comprise nuclease protection. In some aspects, the RNA levels are determined using fluorescence. In some aspects, the RNA levels are determined using an Affymetrix microarray or a microarray such as sold by Agilent.

In some aspects, analyzing the sample for the GPX4 expression level include, but are not limited to, PCR (e.g., real-time PCR), sequencing (e.g., deep sequencing or Next Generation Sequencing, e.g., RNA-Seq), microarray expression profiling, immunohistochemical methods, ELISA, Western analysis, HPLC, proteomics assays, or a combination thereof.

In some aspects, the dihydroorotate dehydrogenase (DHODH) inhibitor is selected from the group consisting of Ag-636, ASLAN003, BAY2402234, leflunomide, brequinar, teriflunomide, IMU-838, PP-001, PTC299, and combinations thereof. In a further aspect, the dihydroorotate dehydrogenase (DHODH) inhibitor is selected from the group consisting of leflunomide, brequinar, teriflunomide, and combinations thereof.

In some aspects, the ferroptosis inducer is selected from the group consisting of sulfasalazine, rosiglitazone, rosiglitazone maleate, bardoxolone methyl, linagliptin, curcumin, zileuton, pioglitazone HCl, nordihydroguaiaretic acid (NDGA), troglitazone, setanaxib, deferoxamine mesylate, sorafenib tosylate, cisplatin, rosadustat, lapatinib, simvastatin, deferasirox, sorafenib, erastin, imidazole ketone erastin, and combinations thereof. In a further aspect, the ferroptosis inducer is selected from the group consisting of sulfasalazine, RSL3, (1S,3R)-RSL3, ML210, ML162, and combinations thereof.

In some aspects, the ferroptosis inducer is not a class (iii) ferroptosis inducer. In some aspects, the DHODH inhibitor is administered in combination with a class (i) ferroptosis inducer. In some aspects, the DHODH inhibitor is administered in combination with a class (ii) ferroptosis inducer. In some aspects, the DHODH inhibitor is administered in combination with a class (iv) ferroptosis inducer.

In some aspects, the subject is a human.

In some aspects, the cancer sample comprises tumor tissue, intratumoral tissue, blood sample, bone marrow, or combinations thereof.

In some aspects, the GPX4 expression levels are determined using sequencing or any technology that measures RNA or protein expression level as, for example, disclosed herein.

The methods disclosed herein can also include additional steps such as prescribing, initiating, and/or altering prophylaxis and/or treatment, based at least in part on the determination of the GPX4 expression levels. In some aspects, the methods disclosed herein further comprise (a) administering chemotherapy; (b) performing surgery; (c) administering radiation therapy; or (d) any combination thereof.

In other aspects, the methods disclosed herein can be combined with standard of care for cancer therapy. In some aspects, standard of care includes, but is not limited to, chemotherapy, radiotherapy, administering immunotherapy, administering targeted therapy, and combination thereof.

In some aspects, the methods disclosed herein reduce the cancer burden.

In some aspects, the cancer burden is reduced by at least about 10%, at least about 20%, at least about 30%, at least about 40%, or about 50% as compared to the cancer burden prior to the administration of the DHODH inhibitor and/or the ferroptosis inducer.

In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration of the DHODH inhibitor and/or the ferroptosis inducer.

In some aspects, the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the DHODH inhibitor and/or the ferroptosis inducer. The term “stable disease” refers to a diagnosis for the presence of a cancer, however the cancer has been treated and remains in a stable condition, i.e. one that that is not progressive, as determined, e.g., by imaging data and/or best clinical judgment. The term “progressive disease” refers to a diagnosis for the presence of a highly active state of a cancer, i.e., one that has not been treated and is not stable or has been treated and has not responded to therapy, or has been treated and active disease remains, as determined by imaging data and/or best clinical judgment.

“Stable disease” can encompass a (temporary) tumor shrinkage/reduction in tumor volume during the course of the treatment compared to the initial tumor volume at the start of the treatment (i.e. prior to treatment). In this context, “tumor shrinkage” can refer to a reduced volume of the tumor upon treatment compared to the initial volume at the start of (i.e. prior to) the treatment. A tumor volume of, for example, less than 100% (e.g., of from about 99% to about 66% of the initial volume at the start of the treatment) can represent a “stable disease.”

“Stable disease” can alternatively encompass a (temporary) tumor growth/increase in tumor volume during the course of the treatment compared to the initial tumor volume at the start of the treatment (i.e. prior to treatment). In this context, “tumor growth” can refer to an increased volume of the tumor upon treatment inhibitor compared to the initial volume at the start of (i.e. prior to) the treatment. A tumor volume of, for example, more than 100% (e.g. of from about 101% to about 135% of the initial volume, preferably of from about 101% to about 110% of the initial volume at the start of the treatment) can represent a “stable disease.”

The term “stable disease” can include the following aspects. For example, the tumor volume does, for example, either not shrink after treatment (i.e. tumor growth is halted) or it does, for example, shrink at the start of the treatment but does not continue to shrink until the tumor has disappeared (i.e. tumor growth is first reverted but, before the tumor has, for example, less than 65% of the initial volume, the tumor grows again.

In some aspects, the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the DHODH inhibitor and/or the ferroptosis inducer.

In some aspects, the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the DHODH inhibitor and/or the ferroptosis inducer.

The term “response” when used herein can refer to a “tumor shrinkage” or a reduction in the number of tumors, for example, when a cancer has metastasized. The term “response” can also be reflected in a “complete response” or “partial response” of the patients or the tumors. The term “complete response” as used herein can refer to the disappearance of all signs of cancer in response to a specific therapy disclosed herein. The term “complete response” and the term “complete remission” can be used interchangeably herein. For example, a “complete response” can be reflected in the continued shrinkage of the tumor (as shown in the appended example) until the tumor has disappeared. A tumor volume of, for example, 0% compared to the initial tumor volume (100%) at the start of (i.e. prior to) the treatment can represent a “complete response.”

Treatment with the DHODH inhibitor and/or the ferroptosis inducer as disclosed herein can result in a “partial response” (or partial remission; e.g. a decrease in the size of a tumor, or in the extent of cancer in the body, in response to the treatment). A “partial response” can encompass a (temporary) tumor shrinkage/reduction in tumor volume during the course of the treatment compared to the initial tumor volume at the start of the treatment (i.e. prior to treatment).

In some aspects, administering the DHODH inhibitor and/or the ferroptosis inducer improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject not receiving the treatment.

In some aspects, administering the DHODH inhibitor and/or the ferroptosis inducer improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not receiving the treatment.

In several aspects of the methods described herein, the samples (e.g., cancer samples or biological samples) can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, the quantification of the expression level of GPX4 disclosed herein, e.g., comparisons between the expression level of a control sample and that of a subject; evaluation of the absence or presence of GPX4; determination of GPX4 expression level with respect to a certain threshold; treatment decisions; or combinations thereof, can be performed by one or more healthcare providers, healthcare benefits providers, and/or clinical laboratories.

As used herein, the term “healthcare provider” refers to individuals or institutions that directly interact with and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a DHODH inhibitor and/or ferroptosis inducer disclosed herein to treat a cancer. A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification of a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition, a healthcare benefits provides can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

### III. Pharmaceutical Compositions

The DHODH inhibitor and/or the ferroptosis inducer can be administered to a subject in the form of a raw chemical without any other components present. The DHODH inhibitor and/or the ferroptosis inducer can also be administered to a subject as part of a pharmaceutical composition containing the compound combined with a suitable pharmaceutically acceptable carrier. Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

Pharmaceutical compositions within the scope of the present disclosure include all compositions where the DHODH inhibitor and/or the ferroptosis inducer is combined with one or more pharmaceutically acceptable carriers. In certain aspects, the DHODH inhibitor and/or the ferroptosis inducer is present in the composition in an amount that is effective to achieve its intended therapeutic purpose. While individual needs may vary, a determination of optimal ranges of effective amounts of each compound is within the skill of the art. Typically, the DHODH inhibitor and/or the ferroptosis inducer, individually or combined, can be administered to a mammal, e.g., a human, at a dose of from about 0.0025 to about 1500 mg per kg body weight of the mammal, or an equivalent amount of a pharmaceutically acceptable salt or solvate thereof, per day to treat the particular disorder. A useful dose of the DHODH inhibitor and/or the ferroptosis inducer, individually or combined, administered to a mammal is from about 0.0025 to about 50 mg per kg body weight of the mammal, or an equivalent amount of the pharmaceutically acceptable salt or solvate thereof. For intramuscular injection, the dose is typically about one-half of the oral dose.

A unit dose may comprise from about 0.01 mg to about 1 g of the DHODH inhibitor and/or the ferroptosis inducer, individually or combined, e.g., about 0.01 mg to about 500 mg, about 0.01 mg to about 250 mg, about 0.01 mg to about 100 mg, 0.01 mg to about 50 mg, e.g., about 0.1 mg to about 10 mg, of the compound. The unit dose can be adminisiered one or more times daily, e.g., as one or more tablets or capsules, each containing from about 0.01 mg to about 1 g of the compound, or an equivalent amount of a pharmaceutically acceptable salt or solvate thereof.

A pharmaceutical composition comprising the DHODH inhibitor and/or the ferroptosis inducer can be administered to any subject, e.g., a cancer patient in need thereof, that may experience the beneficial effects of the DHODH inhibitor and/or the ferroptosis inducer. Foremost among such subjects are mammals, e.g., humans and companion animals, although the disclosure is not intended to be so limited. In one some aspects, the subject is a human.

In some aspects, the DHODH inhibitor can be administered at the same time as the ferroptosis inducer. In other aspects, the DHODH inhibitor can be administered at different times than the ferroptosis inducer. In additional aspects, the DHODH inhibitor and the ferroptosis inducer can be administered sequentially. In an aspect, the DHODH inhibitor can be administered followed by the ferroptosis inducer. In another aspect, the ferroptosis inducer can be administered followed by the DHODH inhibitor.

A pharmaceutical composition of the present disclosure can be administered by any means that achieves its intended purpose. For example, administration can be by the oral, parenteral, subcutaneous, intravenous, intramuscular, intraperitoneal, transdermal, intranasal, transmucosal, rectal, intravaginal or buccal route, or by inhalation. The dosage administered and route of administration will vary, depending upon the circumstances of the particular subject, and taking into account such factors as age, gender, health, and weight of the recipient, condition or disorder to be treated, kind of concurrent treatment, if any, frequency of treatment, and the nature of the effect desired.

In some aspects, a pharmaceutical composition of the present disclosure can be administered orally. In some aspects, a pharmaceutical composition of the present disclosure can be administered orally and is formulated into tablets, dragees, capsules, or an oral liquid preparation. In some aspects, the oral formulation comprises extruded multiparticulates comprising the DHODH inhibitor and/or the ferroptosis inducer.

Alternatively, a pharmaceutical composition of the present disclosure can be administered rectally, and is formulated in suppositories.

Alternatively, a pharmaceutical composition of the present disclosure can be administered by injection.

Alternatively, a pharmaceutical composition of the present disclosure can be administered transdermally.

Alternatively, a pharmaceutical composition of the present disclosure can be administered by inhalation or by intranasal or transmucosal administration.

Alternatively, a pharmaceutical composition of the present disclosure can be administered by the intravaginal route.

A pharmaceutical composition of the present disclosure can contain from about 0.01 to 99 percent by weight, e.g., from about 0.25 to 75 percent by weight, of the DHODH inhibitor and/or the ferroptosis inducer, individually or combined, e.g., about 1%, about 5%, about 10%, about 15%, about 20%, about 25%, about 30%, about 35%, about 40%, about 45%, about 50%, about 55%, about 60%, about 65%, about 70%, or about 75% by weight of the DHODH inhibitor and/or the ferroptosis inducer, individually or combined.

A pharmaceutical composition of the present disclosure is manufactured in a manner which itself will be known in view of the instant disclosure, for example, by means of conventional mixing, granulating, dragee-making, dissolving, extrusion, or lyophilizing processes. Thus, pharmaceutical compositions for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Examples of other pharmaceutical preparations that can be used orally include push-fit capsules made of gelatin, or soft, sealed capsules made of gelatin and a plasticizer such as glycerol or sorbitol. The push-fit capsules can contain a compound in the form of granules, which can be mixed with fillers such as lactose, binders such as starches, and/or lubricants such as talc or magnesium stearate and, optionally, stabilizers, or in the form of extruded multiparticulates. In soft capsules, the active compounds are preferably dissolved or suspended in suitable liquids, such as fatty oils or liquid paraffin. In addition, stabilizers can be added.

Possible pharmaceutical preparations for rectal administration include, for example, suppositories, which consist of a combination of one or more active compounds with a suppository base. Suitable suppository bases include natural and synthetic triglycerides, and paraffin hydrocarbons, among others. It is also possible to use gelatin rectal capsules consisting of a combination of active compound with a base material such as, for example, a liquid triglyceride, polyethylene glycol, or paraffin hydrocarbon.

Suitable formulations for parenteral administration include aqueous solutions of the active compound in a water-soluble form such as, for example, a water-soluble salt, alkaline solution, or acidic solution. Alternatively, a suspension of the active compound can be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

### IV. Kits

In some aspects, the present disclosure provides kits which comprise the DHODH inhibitor and/or the ferroptosis inducer (or a composition comprising the DHODH inhibitor and/or the ferroptosis inducer) packaged in a manner that facilitates their use to practice methods of the present disclosure. In some aspects, the kit includes the DHODH inhibitor and/or the ferroptosis inducer (or a composition comprising the DHODH inhibitor and/or the ferroptosis inducer) packaged in a container, such as a sealed bottle or vessel, with a label affixed to the container or included in the kit that describes use of the compound or composition to practice the method of the disclosure. In some aspects, the compound or composition is packaged in a unit dosage form. The kit further can include a device suitable for administering the composition according to the intended route of administration.

Such kits can comprise various reagents (e.g., in concentrated form) for use in determining the expression level of GPX4, such as GPX4 RNA, GPX4 protein, or both, according to the methods described herein and as known in the art. For example, such reagents can include one or more oligonucleotides (e.g., oligonucleotide capable of hybridizing to an mRNA corresponding to GPX4) or antibodies (e.g., antibodies capable of detecting the protein expression product of GPX4). One or more oligonucleotides or antibodies, e.g., capture antibodies, can be provided already attached to a solid support. One or more oligonucleotides or antibodies can be provided already conjugated to a detectable label. The kits can also provide reagents, buffers, and/or instrumentation to support the practice of the methods provided herein. The kits can also comprise brochures or instructions describing the methods disclosed herein or their practical application to determine a subject's GPX4 expression level. Thus, kits of the present disclosure include kits for determining a subject's GPX4 expression level according to the methods of the disclosure, treating a subject according to the methods of the disclosure, and both determining and treating according to the methods of the disclosure

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: DHODH Regulation of Mitochondrial Lipid Peroxidation and Ferroptosis Induces A Targetable Vulnerability in GPX4^{LOW} Cancer

### Methods and Materials

### Cell Culture Studies

UMRC2, UMRC6, and RCC4 cell lines were provided by W. G. Kaelin at Dana-Farber Cancer Institute. TK-10 cell line was obtained from Dr. Gordon Mills at MD Anderson Cancer Center. All other cancer cell lines were obtained from the American Type Culture Collection. All cell lines were free of mycoplasma contamination (tested by the vendor). No cell line used in this study has been found in the International Cell Line Authentication Committee database of commonly misidentified cell lines, based on short tandem repeat profiling performed by the vendor. Cells were cultured in DMEM with 10% (volume/volume; v/v) FBS and 1% (v/v) penicillin/streptomycin at 37° C. with a humidified atmosphere of 20% O₂ and 5% CO₂. All cell lines were cultured in a 10-cm plate and then cultured into a 12-well plate for cell death and lipid peroxidation measurement. For cell viability assays, cells were cultured into a 96-well plate. Cells were treated with ferroptosis inducers including RSL3 (Selleckchem), ML162 (Cayman Chemical), erastin (Cayman Chemical), FIN56 (Cayman Chemical), or sulfasalazine (Sigma-Aldrich); DHODH inhibitors including brequinar (Tocris), leflunomide (Sigma-Aldrich), or teriflunomide (Sigma-Aldrich); cell death inhibitors liproxstatin-1 (Cayman Chemical) or Z-VAD-FMK (R&D Systems); antioxidants including TEMPO (Sigma-Aldrich), MitoTEMPO (Sigma-Aldrich), MitoQ (Cayman Chemical), or MitoQH₂ (Cayman Chemical); and mitochondria complex III inhibitor including myxothiazol (Sigma-Aldrich).

### Plasmid Constructs

GPX4 short hairpin RNA (shRNA) was purchased from Origene (TR316568). GPX4 expression plasmids were obtained from Dr. Aikseng Ooi at The University of Arizona Health Sciences. DHODH and FSP1 cDNAs were obtained from the Functional Genomics Core Facility of The University of Texas MD Anderson Cancer Center, ciAOX cDNAs was purchased from Addgene (#111661), and subsequently cloned into the lentivirus vector pLVX-Puro. All constructs were confirmed by DNA sequencing.

### CRISPR-Cas9-Mediated Gene Knockout

Knockout of DHODH, ACSL4 and COQ2 in human cell lines was performed using single guide RNAs (sgRNAs) and doxycycline (DOX)-inducible CRISPR-Cas9 expression system as described in our recent publication (Lee, H., et al., Nature cell biology 22, 225-234 (2020)). sgRNAs were cloned into the lentiviral lentiGuide vector. The sequences of sgRNAs used in this study are listed in Table 1. LentiGuide clones were transfected into HEK293T cells with psPAX2 packaging plasmid and pMD2.G expressing plasmid. Cells were infected with lentivirus with 0.8 μg/ml polybrene, selected with puromycin (1 μg/ml, InvivoGen), blasticidin (2 μg/ml, InvivoGen) or hygromycin B (2 μg/ml, InvivoGen) for 3 days and then single cells were sorted into 96-well plates. Single cells were maintained in DMEM with 10% (v/v) FBS and 1% (v/v) penicillin/streptomycin at 37° C. in an incubator with 20% O₂ and 5% CO₂ for 3-4 weeks and each colony was verified by western blot to confirm the target gene deletion. To generate cell lines with DOX-inducible CRISPR-Cas9-mediated gene deletion, cells were infected with lentivirus, selected with puromycin (1 μg/ml, InvivoGen), blasticidin (2 μg/ml, InvivoGen) or hygromycin B (2 μg/ml, InvivoGen) for 2 days and then single cells were sorted into 96-well plates. Once cell lines were established, 1 μg/ml of DOX (Sigma-Aldrich) was added to the media to induce target gene deletion for 48 hours after selection; then cells were cultured in DOX-free media for subsequent analyses.

TABLE 1
Oligo Sequences

SEQ ID NO.

sgRNA oligos sequences

| | | |
|---|---|---|
| Control-sgRNA-F | CACCGGCACTACCAGAGCTAACTCA | SEQ ID NO. 1 |
| Control-sgRNA-R | AAACTGAGTTAGCTCTGGTAGTGCC | SEQ ID NO. 2 |
| DHODH-sgRNA#2-F | CACCGCATCTTATAAAGTCCGTCCA | SEQ ID NO. 3 |
| DHODH-sgRNA#2-R | AAACTGGACGGACTTTATAAGATGC | SEQ ID NO. 4 |
| DHODH-sgRNA#4-F | CACCGTGAGTTGATAAATCCCGGAG | SEQ ID NO. 5 |
| DHODH-sgRNA#4-R | AAACCTCCGGGATTTATCAACTCAC | SEQ ID NO. 6 |
| ACSL4-sgRNA-F | CACCGTGCAATCATCCATTCGGCCC | SEQ ID NO. 7 |
| ACSL4-sgRNA-R | AAACGGGCCGAATGGATGATTGCAC | SEQ ID NO. 8 |
| FSP1-sgRNA-F | CACCGGGAGATGGGGTCCCAGGTCT | SEQ ID NO. 9 |
| FSP1-sgRNA-R | AAACAGACCTGGGACCCCATCTCCC | SEQ ID NO. 10 |
| COQ2-sgRNA-F | CACCGATGCTGGGCTCGCGAGCCGC | SEQ ID NO. 11 |
| COQ2-sgRNA-R | AAACGCGGCTCGCGAGCCCAGCATC | SEQ ID NO. 12 |

shRNA target sequences

| | | |
|---|---|---|
| GPX4 sh#2 | CCGCTGTGGAAGTGGATGAAGATCCAACC | SEQ ID NO. 12 |
| GPX4 sh#3 | GGAAATGCCATCAAGTGGAACTTCACCAA | SEQ ID NO. 14 |

Quantitative Reverse Transcription PCR primer sequences

| | | |
|---|---|---|
| SLC7A11-F | TCCTGCTTTGGCTCCATGAACG | SEQ ID NO. 15 |
| SLC7A11-R | AGAGGAGTGTGCTTGCGGACAT | SEQ ID NO. 16 |
| GPX4-F | ACAAGAACGGCTGCGTGGTGAA | SEQ ID NO. 17 |
| GPX4-R | GCCACACACTTGTGGAGCTAGA | SEQ ID NO. 18 |
| ACSL4-F | GCTATCTCCTCAGACACACCGA | SEQ ID NO. 19 |
| ACSL4-R | AGGTGCTCCAACTCTGCCAGTA | SEQ ID NO. 20 |
| PTGS2-F | CGGTGAAACTCTGGCTAGACAG | SEQ ID NO. 21 |
| PTGS2-R | GCAAACCGTAGATGCTCAGGGA | SEQ ID NO. 22 |
| ACTB-F | CACCATTGGCAATGAGCGGTTC | SEQ ID NO. 23 |
| ACTB-R | AGGTCTTTGCGGATGTCCACGT | SEQ ID NO. 24 |

### Overexpression Cell Lines Generation

Cell lines with stable overexpression of target genes were generated as previously described (Zhang, Y., Koppula, P. & Gan, B, Cell Cycle 18, 773-783 (2019); Chauhan, A. S., et al., FASEB J 33, 2957-2970 (2019)). In brief, HEK293T cells were transfected with either pLVX-empty vector or target gene constructs, together with psPAX.2 and pMD2.G third-generation lentiviral packaging system using 0.8 μg/ml of polybrene. 72 hours later, lentivirus particles in the medium were collected and filtered, then the target cell lines were infected. At 48 hours after infection, puromycin was added to obtain stable cell lines with successful transduction.

### Metabolomic Analysis

Metabolomic analysis was performed as previously described (Liu, X., et al., Nat Cell Biol 22, 476-486 (2020)). Cells were seeded on 35 mm culture plates at a density sufficient to ensure approximately 70-80% confluence at the time of extraction. For metabolic tracer analysis of pyrimidine synthesis, HT-1080 cells were treated with RSL3 and/or Lip-1 for 2 hours and then were shifted to drug-free media containing amide-¹⁵N-glutamine (this time point is defined as 0 hour). Metabolites were extracted at the indicated time points for subsequent analyses. Metabolites were extracted by rapidly aspirating the culture medium and then adding 0.6 mL of an 80% methanol: 20% water mixture at dry ice temperature. The plates were incubated on dry ice for 15 minutes before the cell material was scraped into eppendorf tubes pre-chilled on ice. The cell debris was pelleted by centrifugation at 13,000 RCF for 5 minutes at 4° C. and the supernatant was transferred to a fresh tube and stored on dry ice until analysis. Just prior to analysis, 500 μL of extract was dried under nitrogen gas flow and then resuspended in 100 μL water.

The complete platform consists of an Accela 1250 HPLC system, Accela Open Autosampler, MayLab Mistraswitch column oven and Exactive orbitrap mass spectrometer, controlled by the Xcalibur 3.0.63 software package. Chromatography was performed with a Phenomenex Synergi Hydro-RP column (100×2 mm, 2.5 μm particle size). Solvent A is 10 mM tributylamine and 15 mm acetic acid in water; Solvent B is methanol. The gradient is: 0 min, 0% B; 2.5 min, 0% B; 5 min, 20% B; 7.5 min, 20% B; 13 min, 55% B; 15.5 min, 95% B; 18.5 min, 95% B; 19 min, 0% B; 25 min, 0% B. The injection volume was 10 μL. The column temperature was set to 40° C., and the flow rate was 200 μL/min. The Exactive was operated in negative ionization mode with an electrospray ionization interface. The instrument parameters are as follows: sheath gas flow rate 30 (arbitrary units), aux gas flow rate 10 (arbitrary units), sweep gas flow rate 3 (arbitrary units), spray voltage 3 kV, capillary temperature 325° C., capillary voltage −25 V, tube lens voltage −50 V. The scan range was set to 80-1000 m/z, with a maximum inject time of 250 ms, resolution of 100,000 at 1 Hz, and AGC (automatic gain control) target 1E6. The data were analyzed using the MAVEN software suite57 with signal intensity determined as the Peak Area (Top). For metabolic labeling experiments, the data were corrected to account for the natural abundance of nitrogen-15 using IsoCorrectoR (Heinrich, P., et al., Sci Rep 8, 17910 (2018)).

### Cell Viability Assay

Viable cells were measured using Cell Counting Kit-8 (CCK-8, Dojindo) as previously described (Koppula, P., Zhang, Y., Shi, J., Li, W. & Gan, B, J Biol Chem 292, 14240-14249 (2017); Liu, X. & Gan, B, Cell Cycle 15, 3471-3481 (2016)). Briefly, cells were seeded onto 96-well plates at a density of 2×10⁴ per well. The next day, cells were treated with GPX4 or DHODH inhibitors for 4 hours. Subsequently, cells exposed to 10 μl CCK-8 reagent (100 μl medium per well) for 1 hour at 37° C., 5% CO₂ in an incubator. The absorbance at a wavelength of 450 nm was determined using a FLUOstar Omega microplate reader (BMG Labtech).

### Determination of Lipid Peroxidation

Cells were seeded on 12-well plates and incubated overnight. The next day, cells were treated with compounds for the indicated times, harvested by trypsinization and resuspended in 200 μl PBS containing 5 μM C11-BODIPY 581/591 (Invitrogen). Cells were incubated for 30 min at 37° C. in a water bath. Lipid peroxidation was assessed using the flow cytometer BD Accuri C6 with a 488 nm laser on an FL1 detector. A minimum of 10,000 single cells were analyzed per well.

### Cell Death Assays

Cell death was measured by propidium iodide (Roche) staining using a flow cytometer, as previously described (Dai, F., et al., Proc Natl Acad Sci USA 114, 3192-3197 (2017); Zhang, Y., et al., Cancer Res 80, 2243-2256 (2020)). For propidium iodide-staining, cells were seeded at a density of 50% confluence into 12-well plates. The next day, cells were treated with different reagents. To measure cell death, the cells were collected (including floating dead cells), stained with 5 μg/ml propidium iodide and the percentage of the propidium iodide-positive dead cell population was analyzed using the flow cytometer BD Accuri C6 (BD Biosciences) and an FL2 detector. A minimum of 10,000 single cells were analyzed per well and all experiments were carried out at least in triplicate.

### DHODH Activity Measurement

DHODH activity was determined spectrophotometrically at 37° C. by monitoring the decrease in absorbance at 600 nm of reduced 2,6-dichlorophenol-indophenol (DCPIP) as previously described (Fang, J., et al., Biosci Rep 33, e00021 (2013)). Briefly, the reaction was initiated with 20 mM DHO in 1 ml of standard reaction buffer supplemented with 50 μM DCPIP, 2 μg of rotenone, 2 μg of antimycin A, 5 mM NaN₃ and 0.1 mg of whole-cell lysate. The data were expressed as nmol min⁻¹ μg⁻¹ of protein.

### Glutathione Measurement

Glutathione level measurement was performed as previously described (Zhang, Y., et al., Nat Cell Biol 20, 1181-1192 (2018)) 1×10⁵ cells per well were seeded into 6-well dishes. Cells were treated with drugs for 4 hours. Cells were collected by scraping and prepared for measurement of glutathione using the Glutathione Assay Kit (Cayman Chemical) according to the protocol. The GSH and GSSG concentrations were calculated using a standard curve and normalized to the total protein level.

### CoQ and CoQH₂ Analysis

Ubiquinone (CoQ) and ubiquinol (CoQH₂) were extracted from cultured cells using a modified version of the method developed by Nagase et al. to analyze CoQ and CoQH₂ in cerebrospinal fluid (Nagase, M., Yamamoto, Y., Mitsui, J. & Tsuji, S., J Clin Biochem Nutr 63, 205-210 (2018)). Briefly, cells were first grown on 35 mm plates to approximately 70% confluence. The cells were then quickly washed once in 1 mL of room temperature, serum-free culture medium to remove serum-derived CoQ and CoQH₂ with minimal metabolic perturbation to the cells. 600 μL of freshly prepared, ice-cold isopropanol containing 100 μM tert-butyl-hydroquinone was added to the plates, which were then placed on a cold plate on ice. The cells were scraped with a cell lifter and the cell suspension transferred to Eppendorf tubes on ice. After briefly vortexing, the tubes were clarified by centrifugation at 13,000 RCF at 4° C. for 5 minutes and the supernatants transferred to fresh tubes. These extracts were stored on dry ice in the dark until analysis by LC-MS. The relative levels of CoQ and CoQH₂ in these conditions were stable for at least 48 hours.

CoQ and CoQH₂ levels were determined by LC-MS using an analytical method derived from the lipidomics method as previously described (Zhang, Y., et al., Cell Chem Biol (2019)). The extracts were transferred to amber glass sample vials in an autosampler maintained at 4° C. Separation was performed on a Thermo Scientific UltiMate 3000 HPLC system using an Acquity UPLC HSS T3 column (2.1×100 mm, 1.8 μm particle size). Mobile phase A consisted of acetonitrile:water (60:40, v/v) with 10 mM ammonium acetate and 0.1% acetic acid; mobile phase B consisted of isopropanol:acetonitrile:water (85:10:5, v/v/v) with 10 mM ammonium acetate and 0.1% acetic acid. The gradient was 0 min, 40% B; 1.5 min, 40% B; 12 min, 100% B; 15 min, 100% B; 16 min, 40% B; 17 min, 40% B. The injection volume was 10 μL. The column temperature was set to 55° C., and the flow rate was 400 μL/min.

Samples were analyzed by an Exactive orbitrap mass spectrometer in positive ionization mode with a heated electrospray ion source. The instrument parameters are as follows: sheath gas flow rate 30 (arbitrary units), aux gas flow rate 10 (arbitrary units), sweep gas flow rate 3 (arbitrary units), spray voltage 4 kV, capillary temperature 120° C., heater temperature 500° C., capillary voltage 65 V, tube lens voltage 100 V. The scan range was set to 200-1000 m/z, with a maximum inject time of 100 ms, resolution of 100,000 at 1 Hz, and AGC (automatic gain control) target 1E6. The data were analyzed using the MAVEN software suite 57 with signal intensity determined as the Peak Area (Top). Both CoQ and CoQH₂ were detected as their ammonium adducts ([M+NH₄]+).

### Quantitative Reverse Transcription PCR

qRT-PCR was performed as previously described (Liu, X., et al., Nat Cell Biol 18, 431-442 (2016); Lee, H., et al., Oncotarget 7, 19134-19146 (2016)). Briefly, total RNA was extracted using TRIzol reagent (15596026, Invitrogen), and cDNA was synthesized with SuperScript II Reverse Transcriptase (18064014, Invitrogen). qRT-PCR was performed using SYBR GreenER qPCR SuperMix Universal (11762500, Invitrogen), and triplicate samples were run on a Stratagene MX3000P qPCR system according to the manufacturer's protocol. The threshold cycle (Ct) values for each gene were normalized to those of β-actin, and the 2^{-ΔΔCt} method was used for quantitative analysis. Primer sequences are listed in Table 1.

### Immunoblotting

Western blotting to analyze protein expression was performed as previously described (Lin, A., et al., Oncogene 33, 3183-3194 (2014); Lin, A., et al., Cancer Res 74, 1682-1693 (2014)). Briefly, cell pellets and tissues were lysed using IP lysis buffer (Fisher Scientific) and the protein concentration was determined by a Bicinchoninic Acid Protein Assay (Thermo Scientific) using a FLUOstar Omega microplate reader (BMG Labtech). 25 μg of protein was used for immunoblot analysis using antibodies against DHODH (1:1,000, 14877-1-AP, Proteintech), GPX4 (1:1,000, MAB5457, R&D systems), vinculin (1:3,000, V4505, Sigma-Aldrich), SLC7A11 (1:1,000, 12691, Cell Signaling), ACSL4 (1:1,000, sc-271800, Santa Cruz), COQ2 (1:1,000, sc-517107, Santa Cruz Biotechnology), FSP1 (1:1,000, sc-377120, Santa Cruz Biotechnology), and Flag (1:10,000, 66008-3-Ig, Proteintech).

### Histology and Immunohistochemistry

Fresh tumor tissues were fixed in 10% neutral buffered formalin overnight, washed once with PBS, and stored in 70% ethanol at 4° C. The tissues were dehydrated and embedded in paraffin by the Research Histology Core Laboratory (MD Anderson Cancer Center) according to standard protocols. Embedded tissues were sectioned at a thickness of 5 μm for H&E or immunohistochemistry (IHC) analysis. IHC analysis was performed as previously described (Gan, B., et al., Cancer Cell 18, 472-484 (2010); Gan, B., et al., J Cell Biol 175, 121-133 (2006)). Briefly, the primary antibodies, including ki-67 (1:500, 9027s, Cell Signaling Technology), cleaved-caspase 3 (1:500, 9661s, Cell Signaling Technology), or 4-HNE (1:400, ab46545, Abcam) were incubated overnight at 4° C. Staining was performed using the Vectastain elite ABC kit and DAB peroxidase substrate kit (Vector laboratories). Images were randomly taken from the renal cortex (five images per tumor) at ×200 magnification using an Olympus BX43 microscope.

### Cell Line-Derived Xenograft Model

All the xenograft experiments were performed in accordance with a protocol approved by the Institutional Animal Care and Use Committee and Institutional Review Board at The University of Texas MD Anderson Cancer Center. The study is compliant with all relevant ethical regulations regarding animal research. Female 4- to 6-week-old athymic nude mice (Foxn1^{nu}/Foxn1^{nu}) were purchased from the Experimental Radiation Oncology Breeding Core Facility at MD Anderson Cancer Center and housed in the Animal Care Facility at the Department of Veterinary Medicine and Surgery at MD Anderson. Cancer cell lines were suspended and counted in cold phosphate-buffered saline (PBS), and 5×10⁶ HT-1080 or 1×10⁷ NCI-H226 cells were injected into mice subcutaneously. When the tumor reached 50-100 mm³, the mice were assigned randomly into different treatment groups. Brequinar or sulfasalazine was dissolved in dimethyl sulfoxide (DMSO) and diluted in PBS. Brequinar was intraperitoneally injected into mice at a dose of 30 mg/kg every three days. Sulfasalazine was intraperitoneally injected daily at a dose of 100 mg/kg. Liproxstatin-1 diluted in PBS was intraperitoneally injected daily at a dose of 10 mg/kg. The daily injection of brequinar, sulfasalazine, or liproxstatin-1 was continued until the endpoint as indicated in the corresponding figures. The tumor volume was measured every 3 days until the endpoint and calculated according to the equation volume=length×width²×½.

### Patient-Derived Xenograft (PDX) Model

PDXs were generated in accordance with protocols approved by the Institutional Review Board at The University of Texas MD Anderson Cancer Center. Informed consent was obtained from the patients and the study is compliant with all relevant ethical regulations regarding research involving human participants. All the NOD scid gamma (NSG) mice were purchased from the Experimental Radiation Oncology Breeding Core Facility at MD Anderson Cancer Center and housed in the Animal Care Facility at the Department of Veterinary Medicine and Surgery at MD Anderson Cancer Center. PDX model used in this study was originally obtained from lung cancer PDX platform at MD Anderson Cancer Center. PDX experiments were performed as previously described (Liu, X., et al., Nat Cell Biol 22, 476-486 (2020)). Briefly, PDX tumors in cold DMEM media were minced into fragments 1-2 mm³ in volume. Then each PDX tumor fragment was subcutaneously inoculated into the dorsal flank of NSG mice. When the tumors reached 50-100 mm 3 in volume, the mice were assigned randomly into different treatment groups for the treatment of brequinar, sulfasalazine, and/or liproxstatin-1 as described under cell line-derived xenograft model. The tumor volume was measured every 3 days until the endpoint and calculated according to the equation volume=length×width²×½.

### Statistics and Reproducibility

Results of cell culture experiments were collected from at least 3 independent replicates. Volumes or weights from at least 6 tumor in each group were plotted. Data are presented as means±standard deviation (SD). Statistical significance (P values) was calculated using unpaired Student's t-tests or log-rank test by GraphPad Prism 8.0 or SPSS 25.0. *P<0.05; **P<0.01; ***P<0.001; ****P<0.0001; ns., non-significant.

### Results

### Metabolomic Analysis Links DHODH to Ferroptosis Regulation

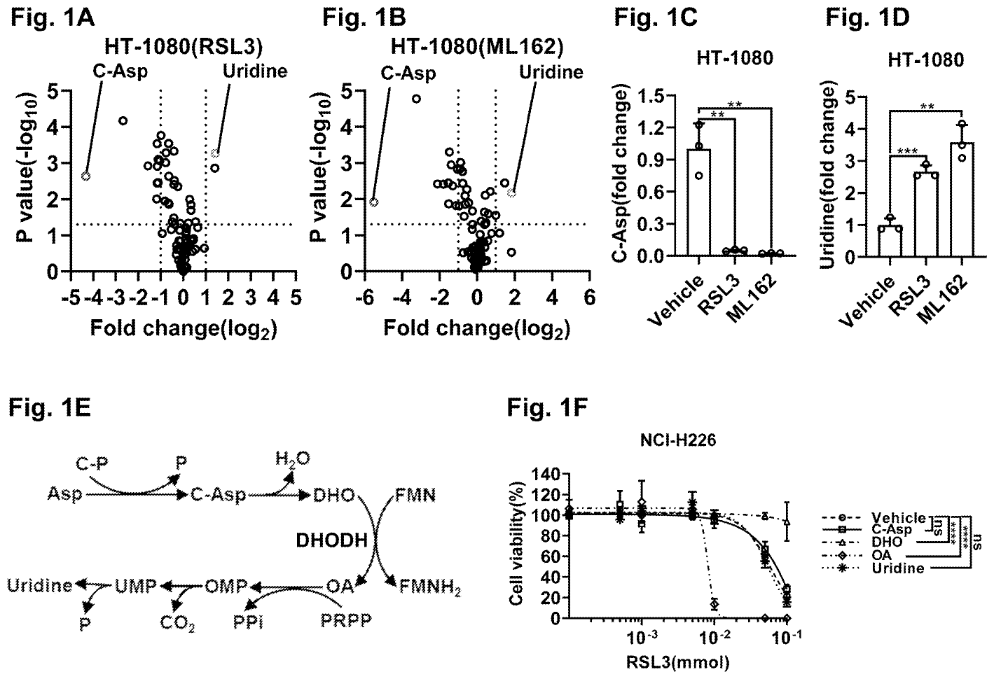

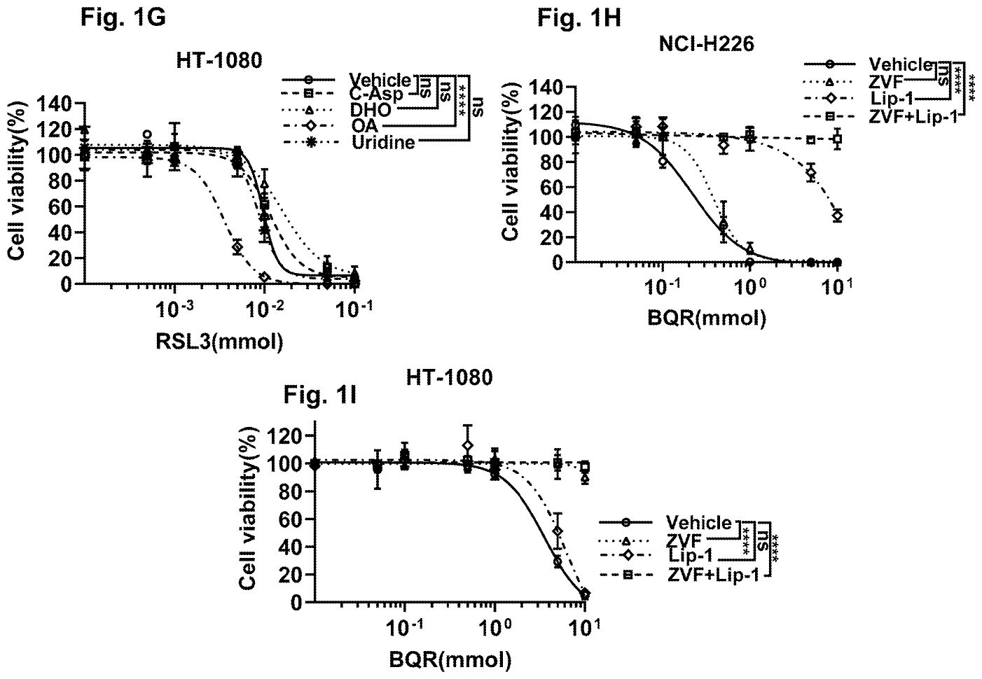

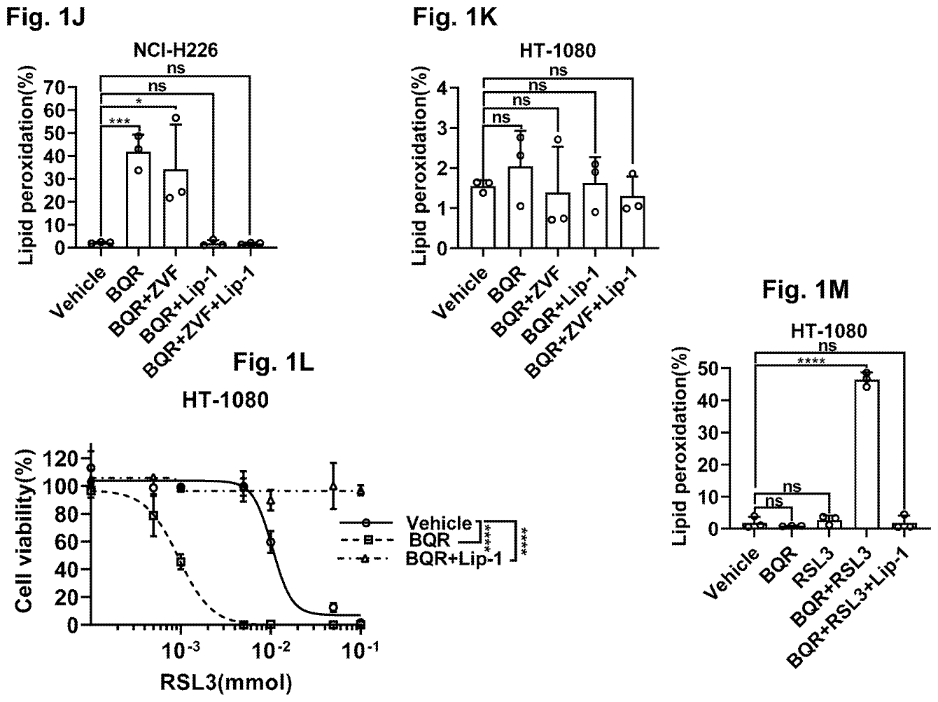

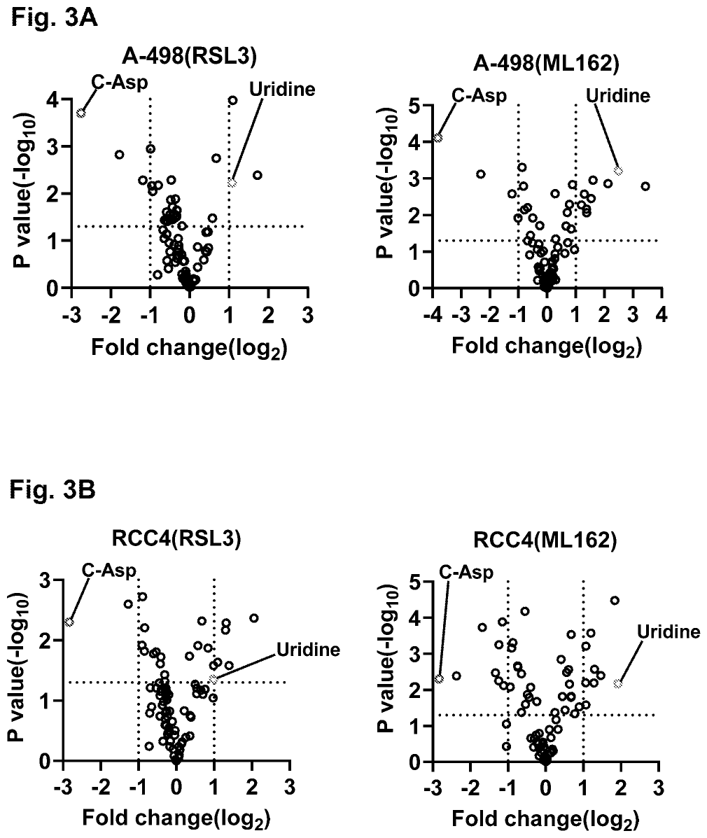

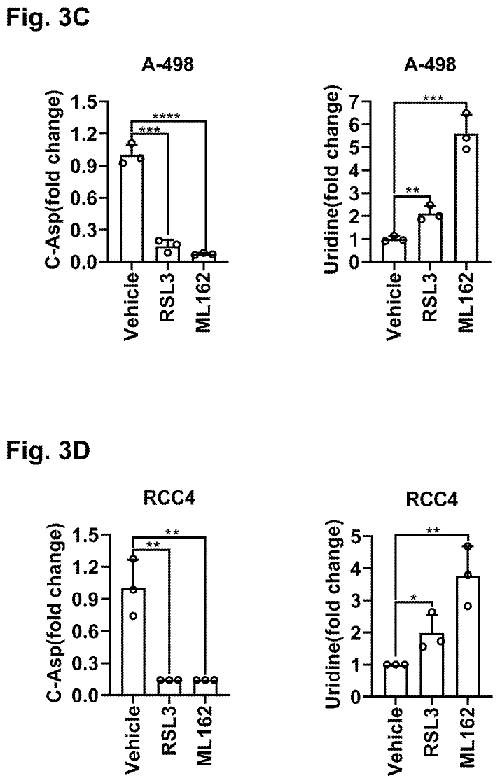

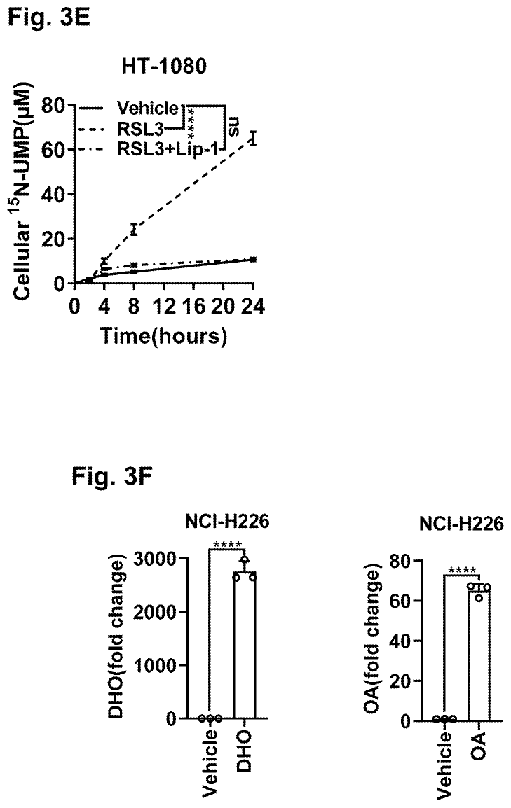

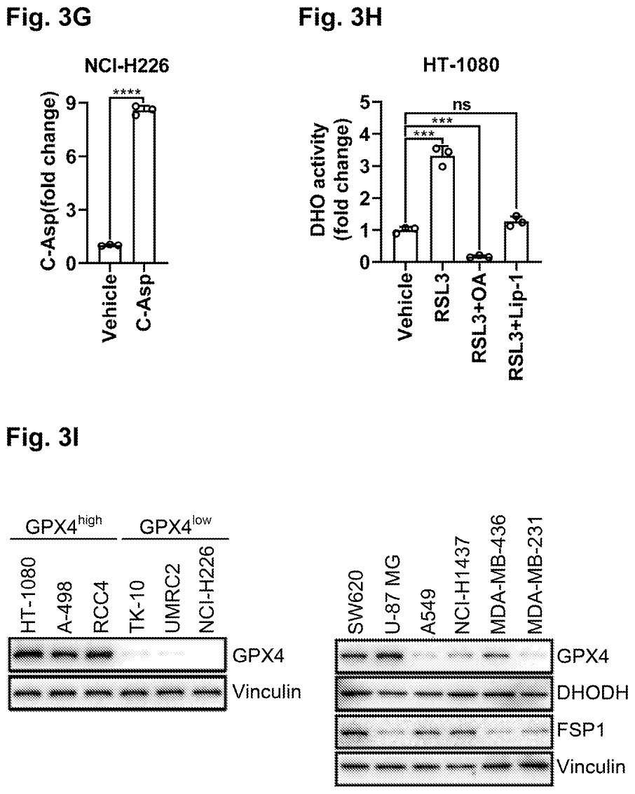

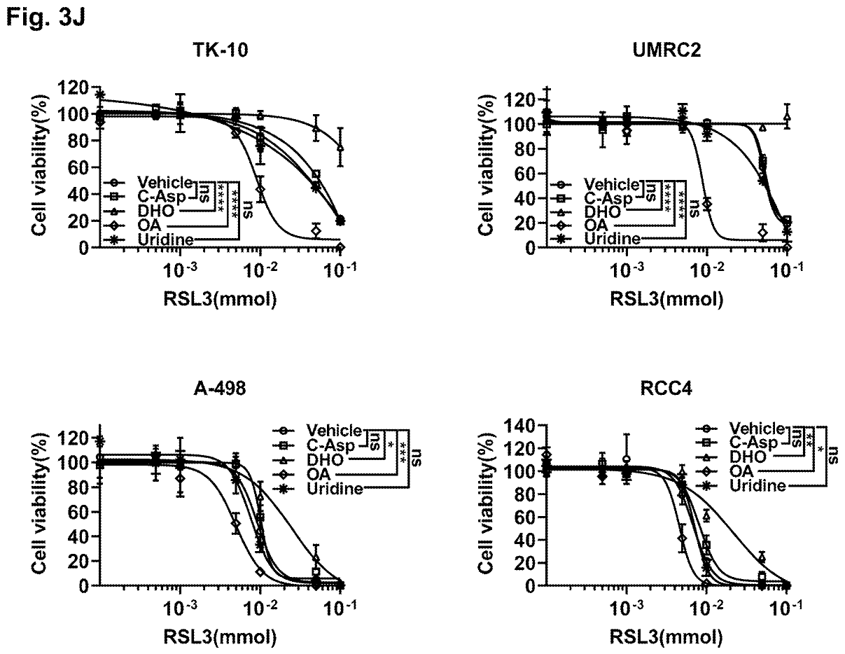

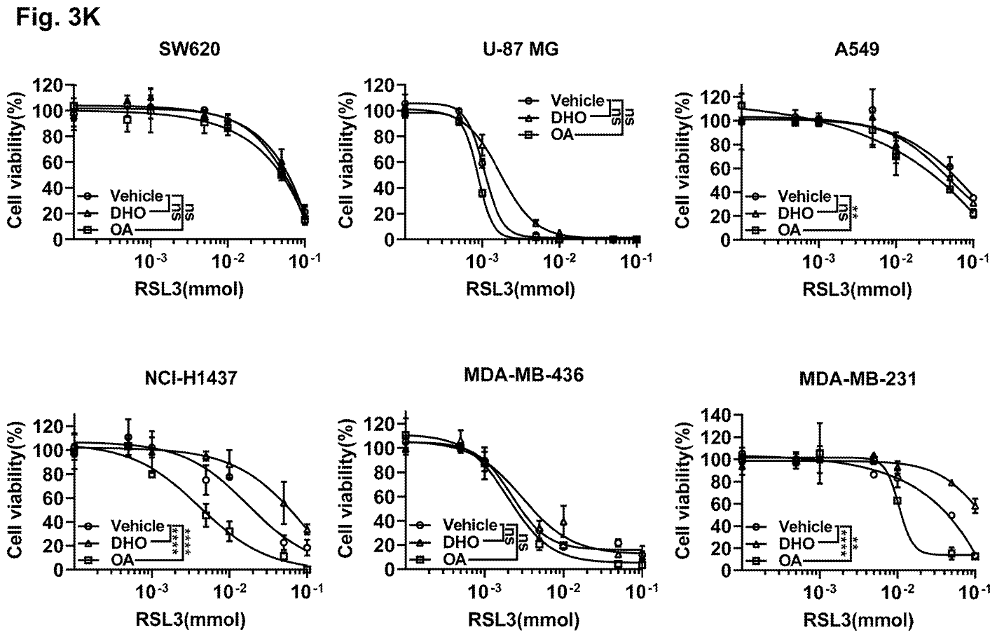

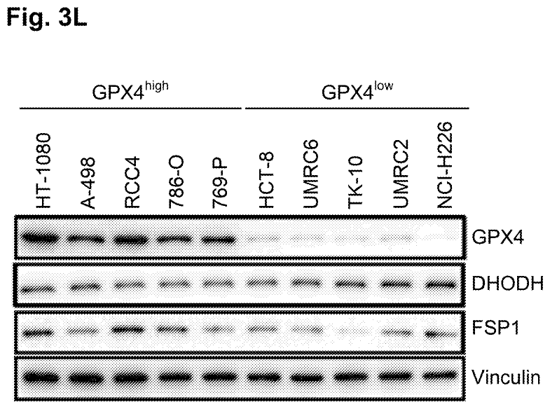

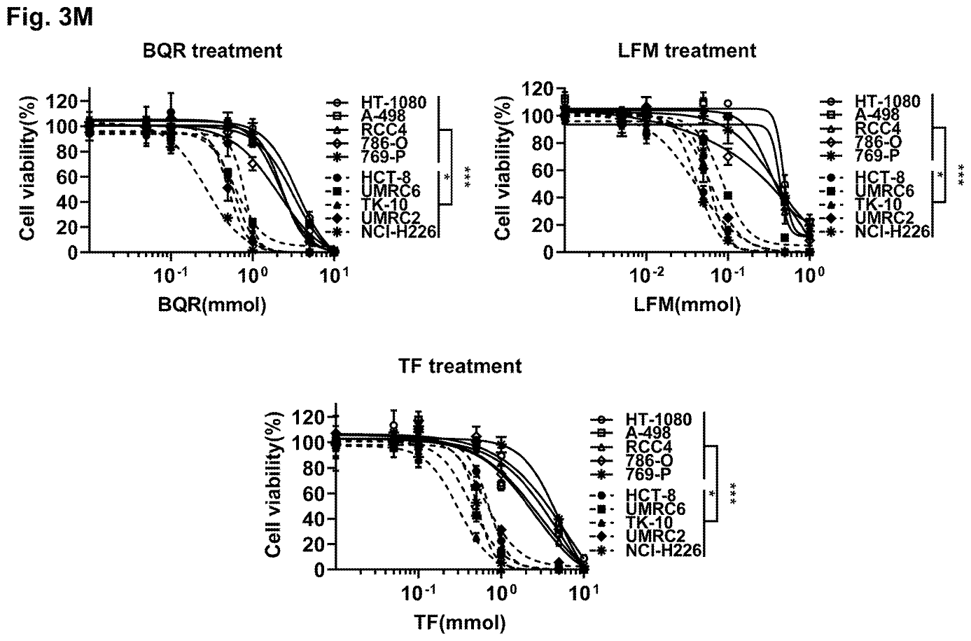

Global metabolomic analyses revealed that short-term (2-hour) treatment with GPX4 inhibitor RSL3 or ML162 in multiple cancer cell lines resulted in a marked depletion of C-Asp, an intermediate of pyrimidine biosynthesis, with a concomitant accumulation of uridine, an end product of pyrimidine biosynthesis (FIG. 1A, FIG. 1B, FIG. 1C, FIG. 1D, FIG. 1E, FIG. 3A, FIG. 3B, FIG. 3C, and FIG. 3D). Metabolic tracer studies using glutamine labeled at the amide nitrogen, which is incorporated into the pyrimidine ring in the first step of the de novo biosynthetic pathway, revealed that RSL3 treatment significantly increased ¹⁵N-UMP levels, and this effect was completely rescued by the ferroptosis inhibitor liproxstatin-1 (FIG. 3E). Without wishing to be bound by theory, as this metabolic alteration might reflect an attempt to restore cellular defense systems against ferroptosis in response to acute GPX4 inactivation, the potential effects of supplementing pyrimidine biosynthesis intermediates on ferroptosis induced by GPX4 inhibition (FIG. 1E) was tested. These analyses revealed that DHO protected cells from, whereas OA sensitized cells to, GPX4 inhibition; surprisingly, uridine supplementation did not affect cellular sensitivity to GPX4 inhibitors (FIG. 1F). It can be seen that DHO or OA supplementation drastically increased its intracellular levels (FIG. 3F). C-Asp appears to be largely impermeable to cells, as C-Asp supplementation only moderately increased its intracellular levels (FIG. 3G) and consequently did not affect ferroptosis sensitivity to RSL3 (FIG. 1F). That the substrate and product of the DHODH reaction (FIG. 1E) exerted opposite effects on ferroptosis sensitivity suggested a potential role for DHODH in regulating ferroptosis in a manner independent of its role in generating pyrimidine nucleotides. Consistent with this, it was observed that RSL3-induced ferroptotic stress significantly increased DHODH activity (FIG. 3H).

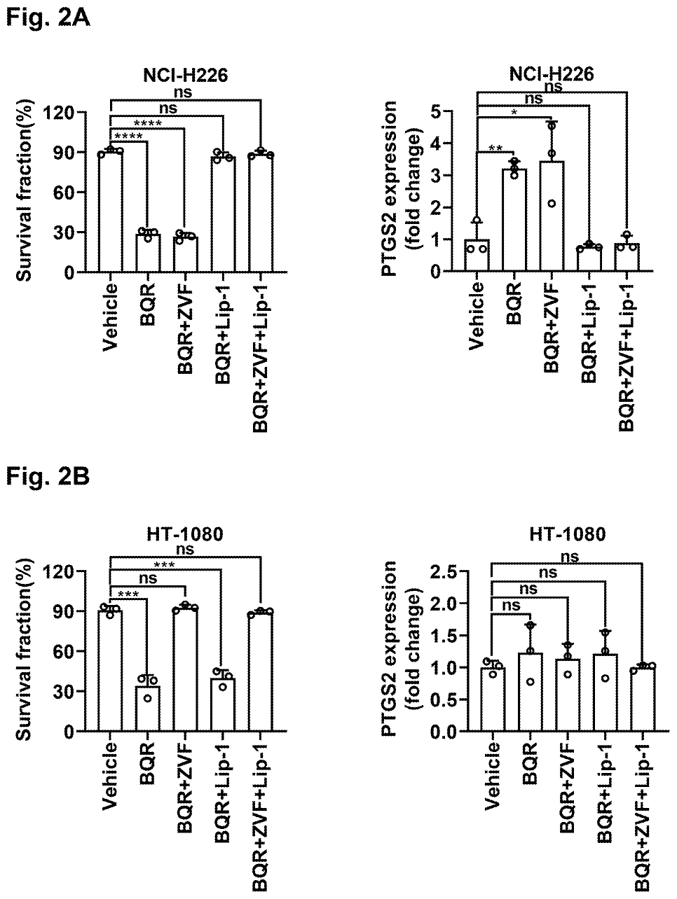

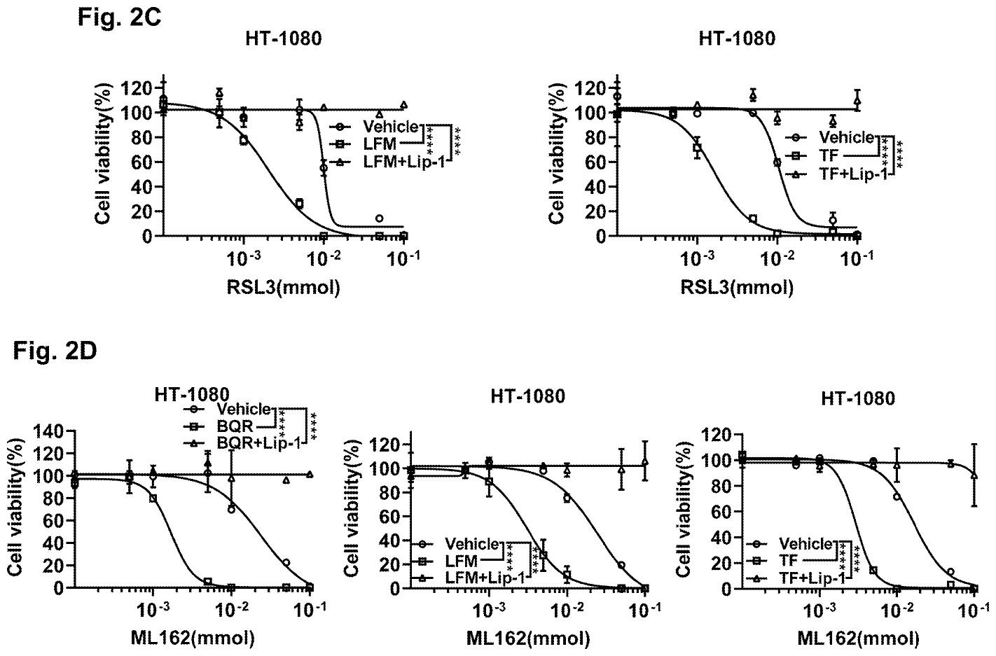

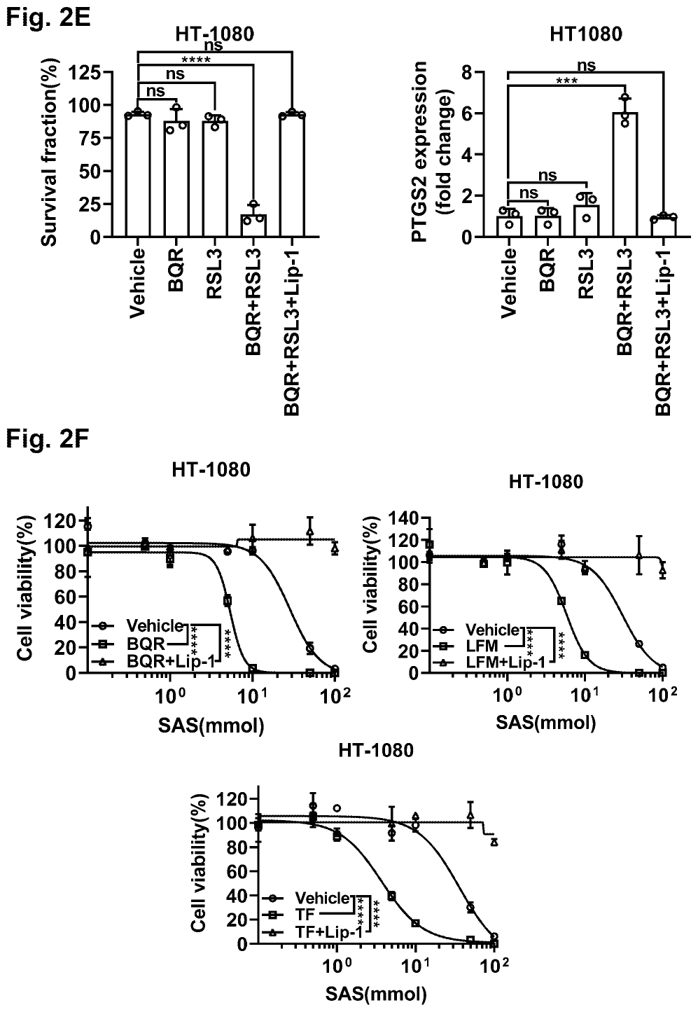

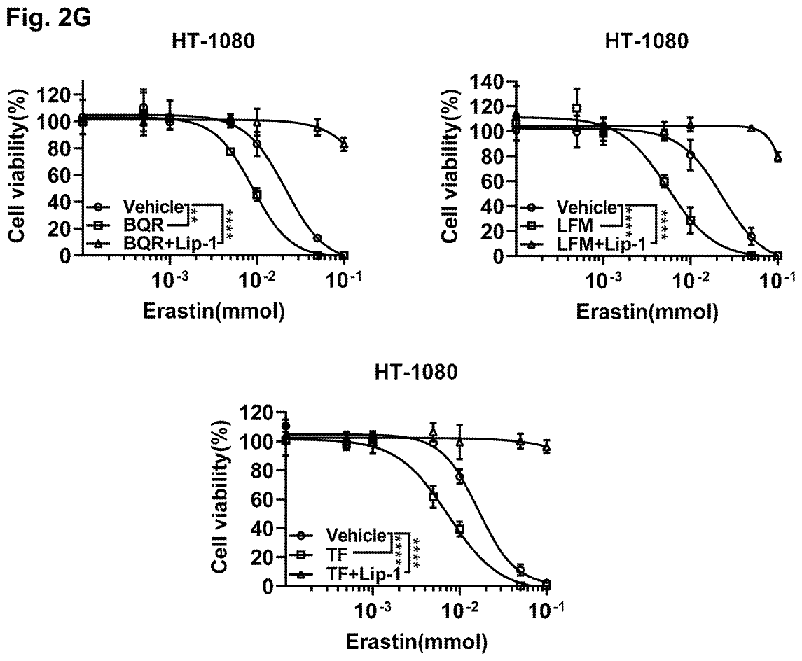

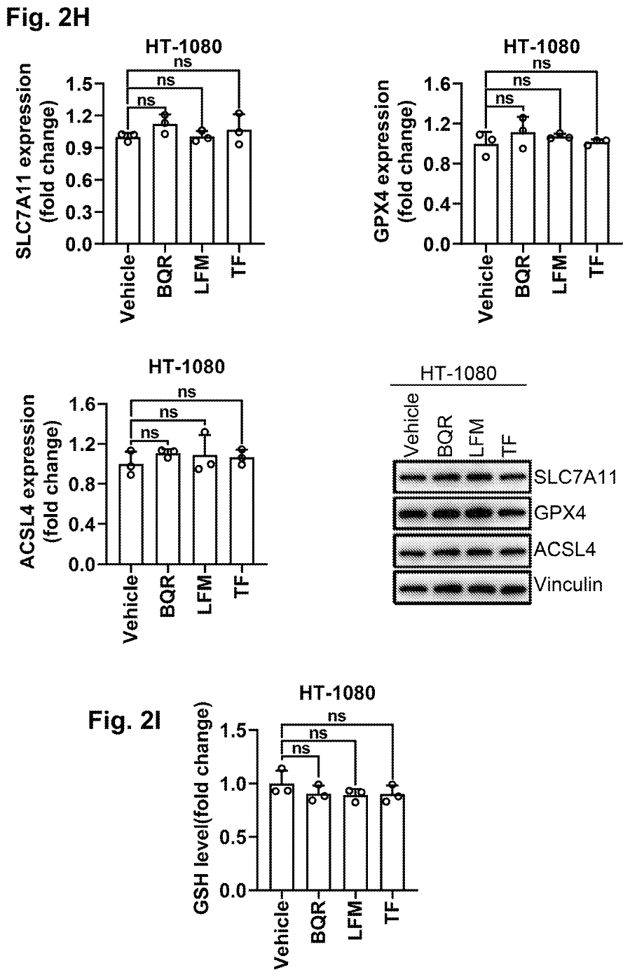

It can be seen that DHO (or OA) supplementation generally exerted much more pronounced protecting (or sensitizing) effects in GPX4^{low} cell lines (such as NCI-H226) than in GPX4^{high} cell lines (such as HT-1080) (FIG. 1F, FIG. 1G, FIG. 3I, FIG. 3J, and FIG. 3K). Analyses of a panel of GPX4^{high} and GPX4^{low} cancer cell lines revealed that GPX4^{low} cancer cells generally were more sensitive to DHODH inhibitors (FIG. 3L and FIG. 3M). Liproxstatin-1, but not the apoptosis inhibitor Z-VAD-FMK, largely rescued DHODH inhibition-induced cell death in GPX4^{low} NCI-H226 cells (FIG. 1H and FIG. 2A), whereas the opposite was observed in GPX4^{high} HT-1080 cells treated with a DHODH inhibitor (FIG. 1I and FIG. 2B). DHODH inhibition induced potent lipid peroxidation and ferroptosis marker gene PTGS2 expression in GPX4^{low} but not in GPX4^{high} cancer cells (FIG. 1J, FIG. 1K, FIG. 2A, and FIG. 2B). These data suggest that DHODH inhibition primarily induces ferroptosis in GPX4^{low} cancer cells, and that ferroptosis induction likely accounts for the more pronounced vulnerability of GPX4^{low} cancer cells than GPX4^{high} cancer cells to DHODH inhibition. While DHODH inhibition did not induce ferroptosis in GPX4^{high} HT-1080 cells (FIG. 1I), it markedly sensitized such cells to ferroptosis induced by class 2 FINs (RSL3 and ML162, which inhibit GPX4 activity) (FIG. 1L, FIG. 1M, FIG. 2C, FIG. 2D, and FIG. 2E) or class 1 FINs (sulfasalazine and erastin, which block SLC7A11-mediated cystine transport; cancer cells mainly rely on SLC7A11 to obtain cysteine for GSH synthesis (Koppula, P., Zhuang, L. & Gan, B., Protein Cell (2020)) (FIG. 2F and FIG. 2G). DHODH inhibitor treatment did not affect the expression of GPX4, SLC7A11, or ACSL4 (a lipid metabolism enzyme that is required for ferroptosis in many cell lines (Doll, S., et al., Nature chemical biology 13, 91-98 (2017)), or GSH levels (FIG. 2H and FIG. 2I). Together, these data suggest that DHODH inhibition induces ferroptosis in GPX4^{low} cancer cells but sensitizes GPX4^{high} cancer cells to ferroptosis, and that DHODH regulation of ferroptosis is likely independent of (or in parallel to) the SLC7A11-GPX4 signaling.

### DHODH Deletion Promotes Ferroptosis

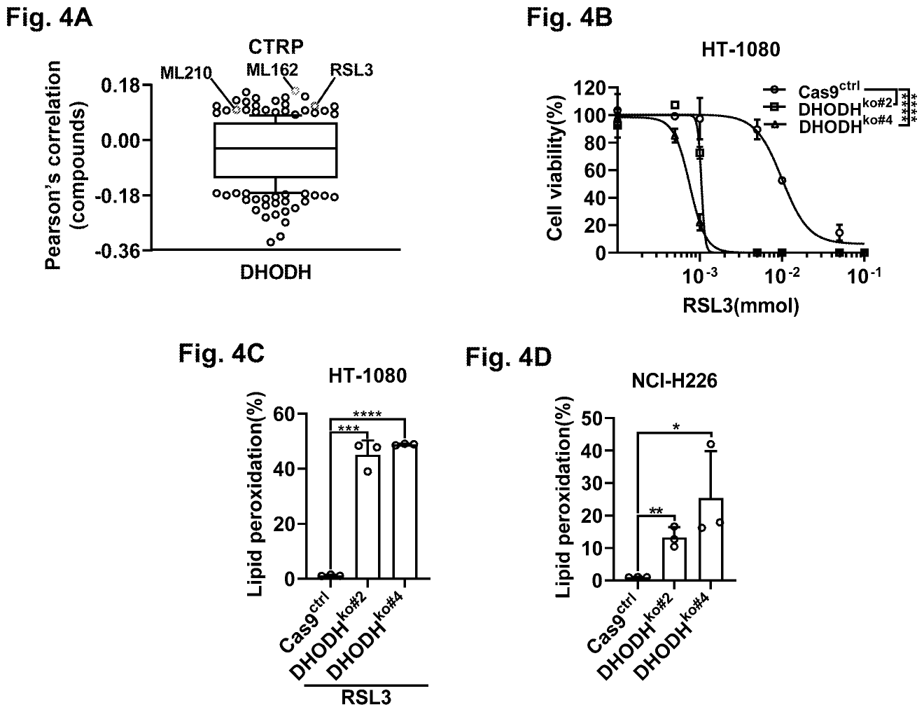

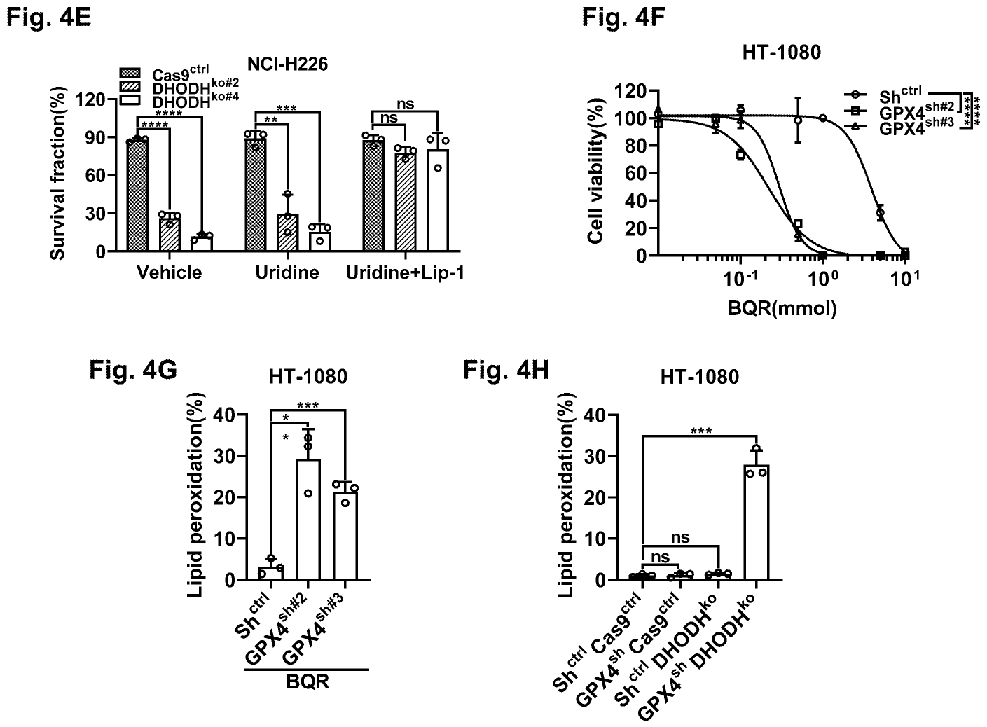

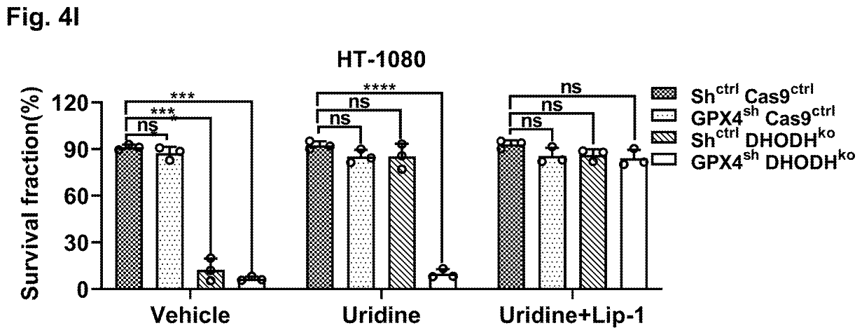

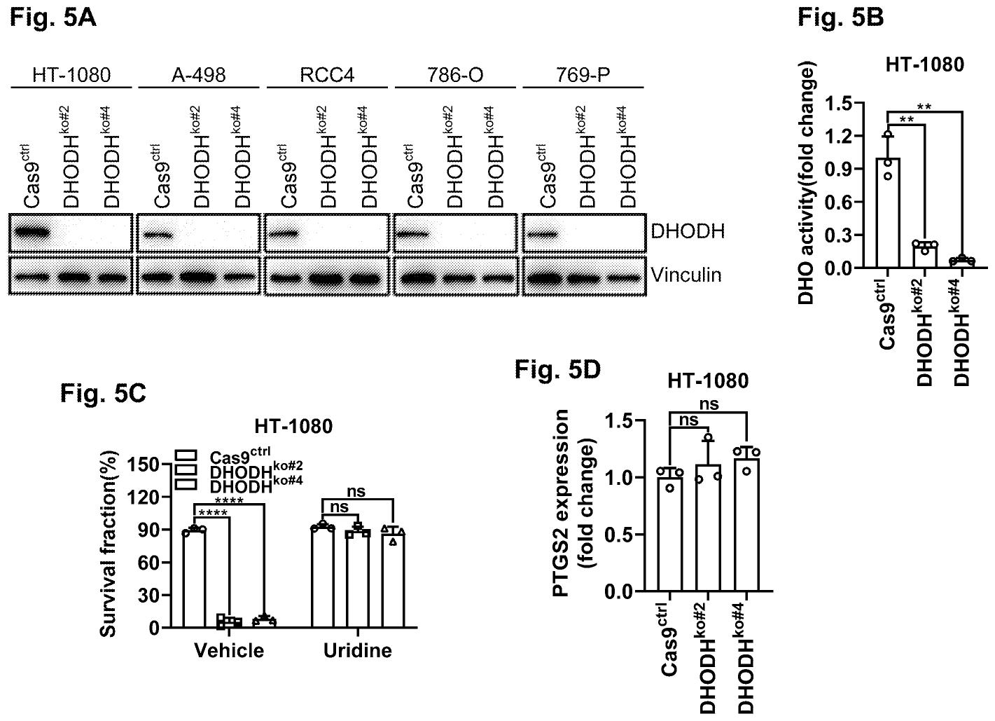

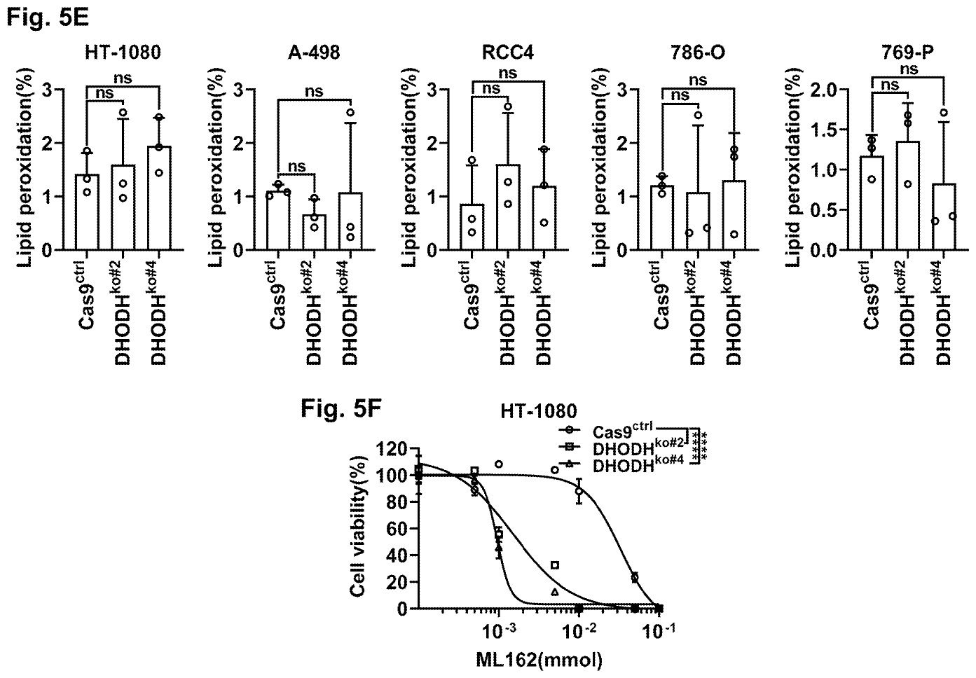

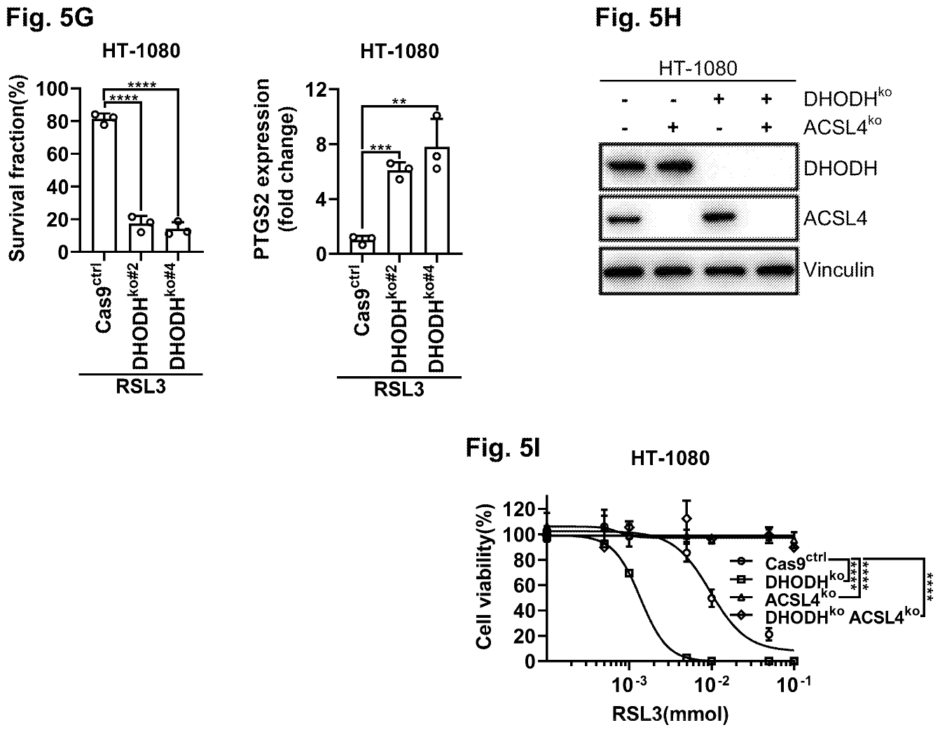

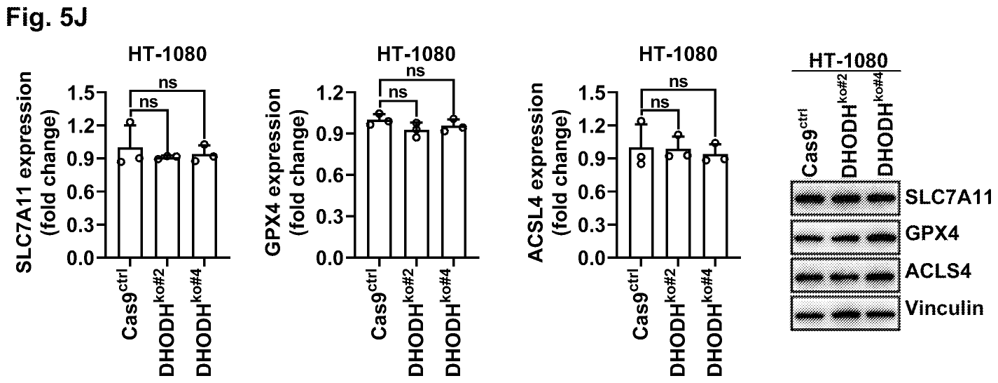

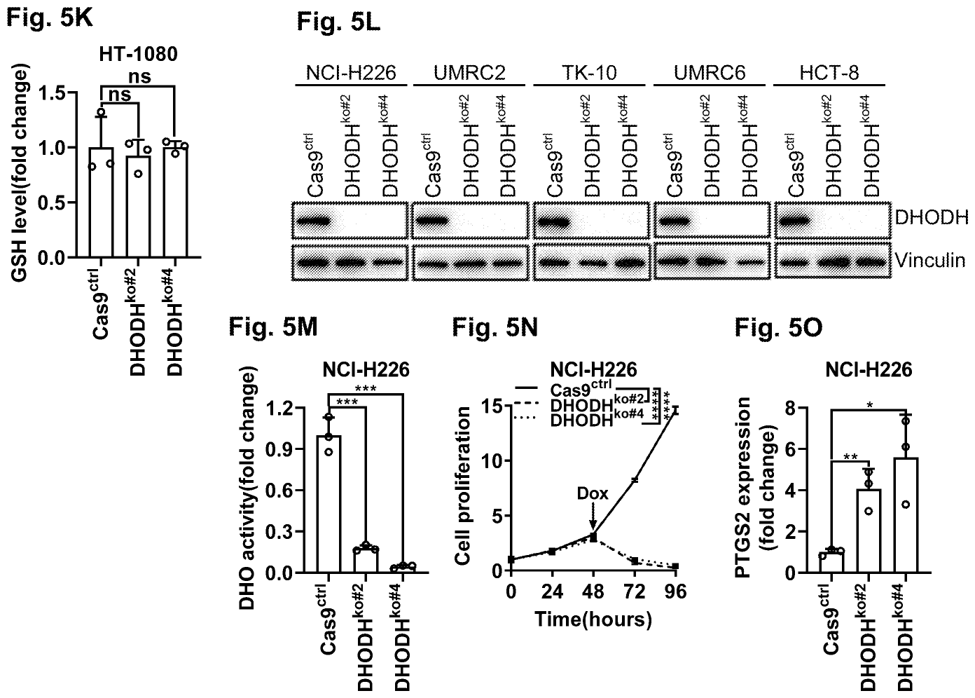

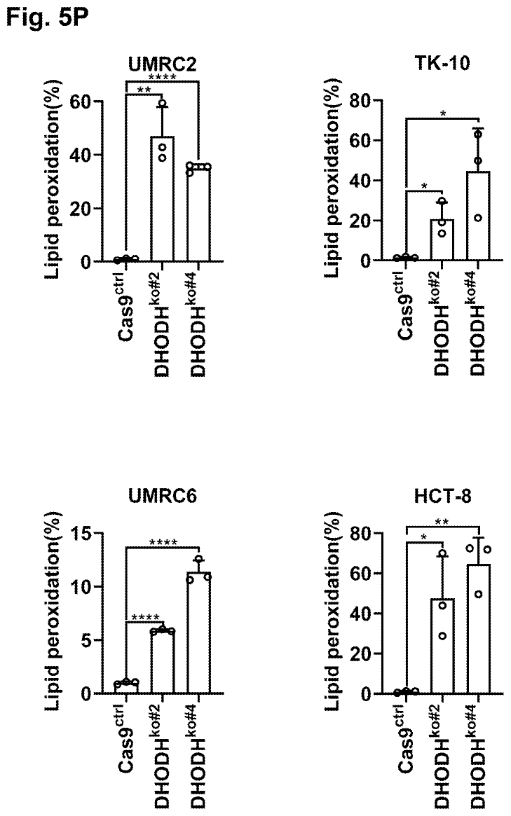

A genetic approach was used to study DHODH function in ferroptosis regulation. Analyses of the Cancer Therapeutics Response Portal (Basu, A., et al., Cell 154, 1151-1161 (2013)) revealed that, among more than 500 drugs, GPX4 inhibitors exhibit some of the strongest resistance correlations to DHODH expression (FIG. 4A). DHODH knockout (KO) in GPX4^{high} HT-1080 cells by an inducible CRISPR-Cas9 approach (FIG. 5A) abolished DHODH enzyme activity and induced substantial cell death (but without inducing obvious ferroptosis marker gene PTGS2 expression or lipid peroxidation), which could be largely rescued by uridine supplementation (FIG. 5B, FIG. 5C, FIG. 5D, and FIG. 5E). The lack of lipid peroxidation induction upon DHODH deletion was confirmed in additional GPX4^{high} cancer cells (FIG. 5A and FIG. 5E). (Unless otherwise stated, uridine supplemented medium was used in all studies with DHODH KO cells.) Under uridine-supplemented conditions, DHODH deletion markedly sensitized HT-1080 cells to RSL3- or ML162-induced lipid peroxidation and ferroptosis (FIG. 4B, FIG. 4C, FIG. 5F, and FIG. 5G), which could be largely abolished by ACSL4 deletion (FIG. 5H and FIG. 5I). DHODH deletion did not affect GPX4, SLC7A11, or ACSL4 expression levels, or GSH levels in HT-1080 cells (FIG. 5J and FIG. 5K). Unlike in HT-1080 cells, DHODH deletion in GPX4^{low} NCI-H226 cells potently induced lipid peroxidation and ferroptosis even with uridine supplementation, and uridine supplementation was not sufficient to maintain long-term culturing of DHODH KO NCI-H226 cells (FIG. 4D, FIG. 4E, FIG. 5L, FIG. 5M, FIG. 5N, and FIG. 5O). DHODH deletion-induced lipid peroxidation in additional GPX4^{low} cancer cells (FIG. 5L and FIG. 5P) was validated. Therefore, the data with genetic ablation of DHODH mirrored the data using DHODH inhibitors (FIG. 1H, FIG. 1I, FIG. 1J, FIG. 1K, FIG. 1L, and FIG. 1M).

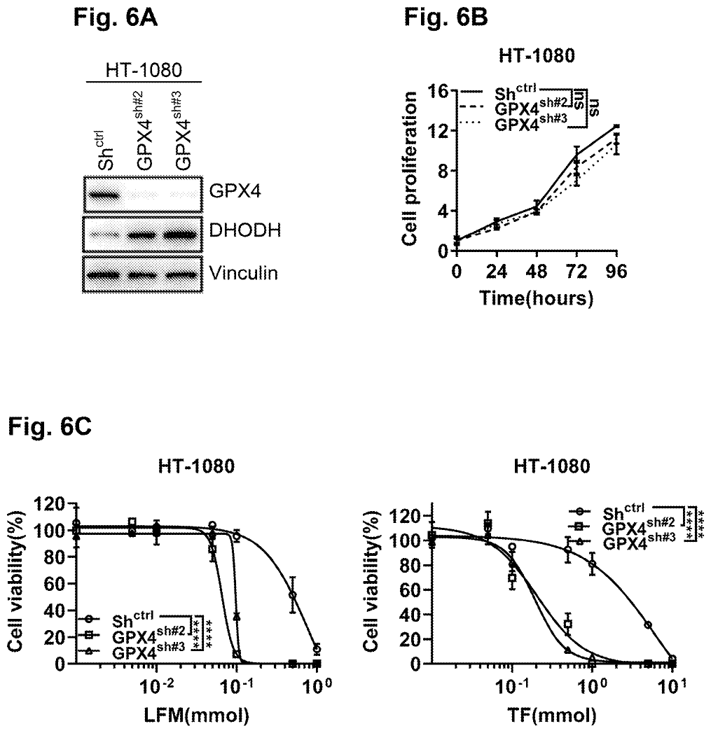

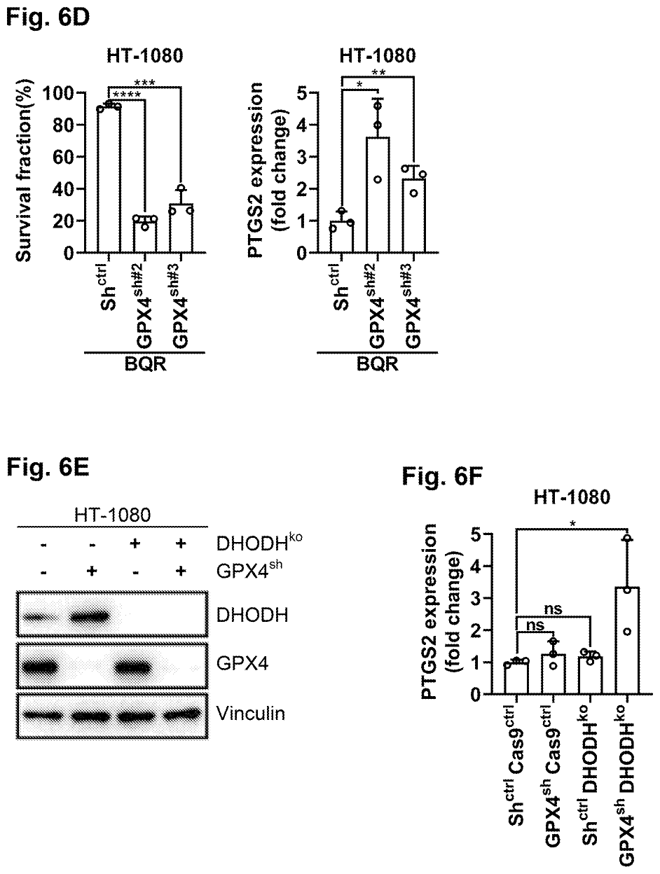

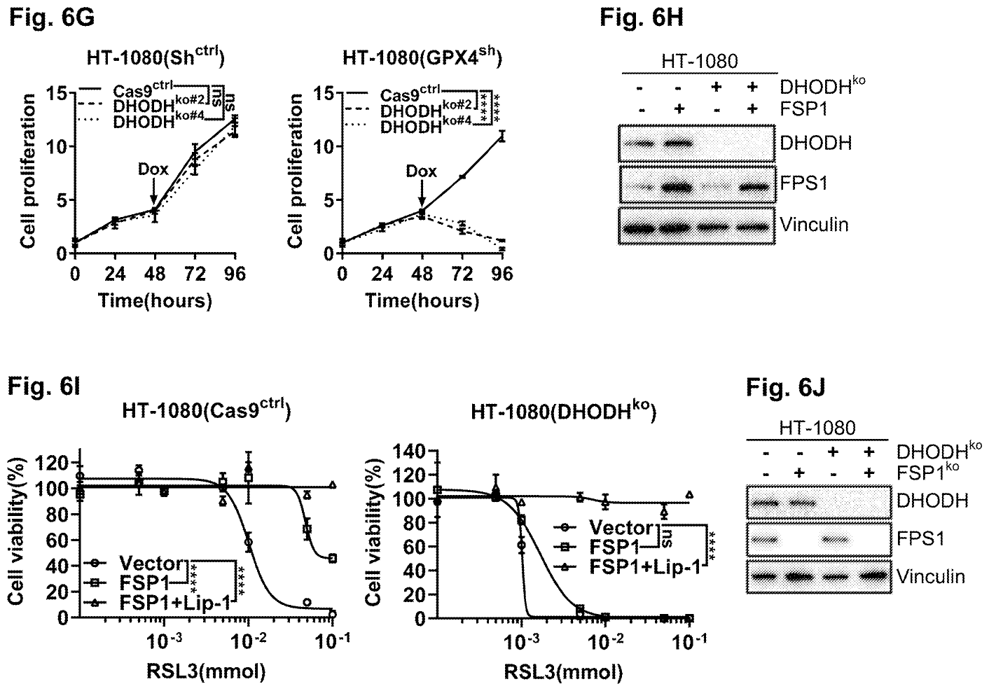

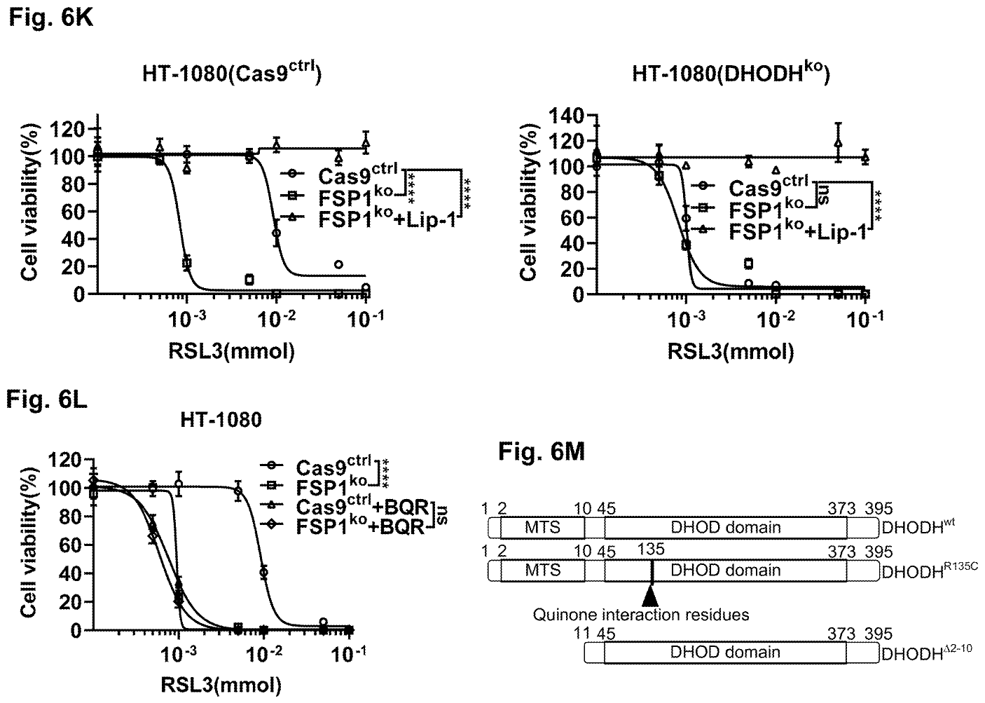

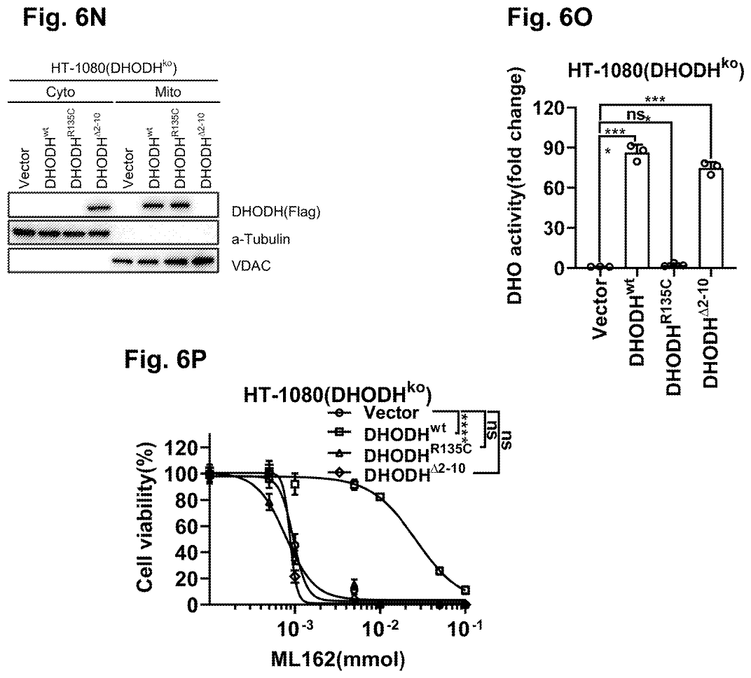

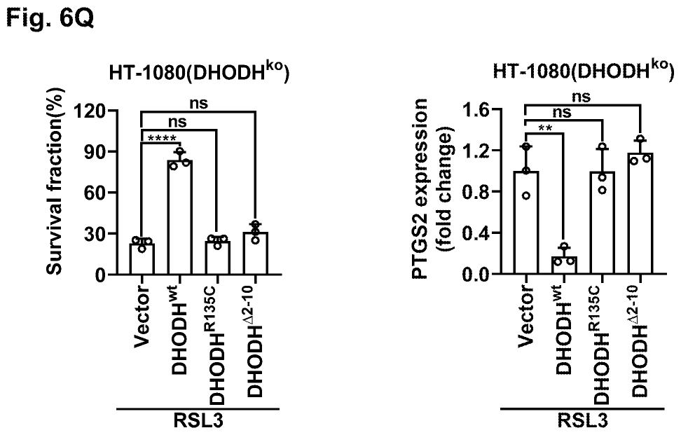

The aforementioned data prompted further analyses of the genetic interaction between DHODH and GPX4. GPX4 partial knockdown in HT-1080 cells did not significantly affect basal cell growth or viability but markedly sensitized cells to DHODH inhibitor-induced lipid peroxidation and ferroptosis (FIG. 4F, FIG. 4G, FIG. 6A, FIG. 6B, FIG. 6C, and FIG. 6D). GPX4 knockdown in HT-1080 cells significantly increased DHODH levels (FIG. 6A), which likely represents an adaptive cellular response attempting to suppress ferroptosis in response to chronic GPX4 inactivation. Further deleting DHODH in GPX4 knockdown HT-1080 cells (but not DHODH deletion or GPX4 knockdown alone) markedly induced lipid peroxidation and ferroptosis, which could be rescued by ferroptosis inhibitor liproxstatin-1 treatment (FIG. 4H, FIG. 4I, FIG. 6E, and FIG. 6F); of note, uridine supplementation could rescue cell death in DHODH KO HT-1080 cells, but not in such cells with GPX4 knockdown (FIG. 4I and FIG. 6G). Together, the data suggest that DHODH acts in parallel to GPX4 to suppress ferroptosis, and DHODH deletion induces ferroptosis in GPX4^{low} cancer cells (or GPX4^{high} cancer cells with GPX4 knockdown).

The genetic interaction between DHODH and FSP1 was studied (Bersuker, K., et al., Nature 575, 688-692 (2019); Doll, S., et al., Nature 575, 693-698 (2019)). FSP1 overexpression significantly suppressed, while its deletion promoted, RSL3-induced ferroptosis in HT-1080 cells; however, FSP1 overexpression or deletion did not affect RSL3-induced ferroptosis in DHODH KO cells (FIG. 6H, FIG. 6I, FIG. 6J, and FIG. 6K). Likewise, treatment with the DHODH inhibitor brequinar promotes RSL3-induced ferroptosis in wild-type (WT) cells but not in FSP1 KO cells (FIG. 6L). Thus, DHODH and FSP1 operate in two separate systems to inhibit ferroptosis, consistent with the distinctive localization of these two proteins on mitochondria inner membrane and plasma membrane, respectively.

### DHODH Suppresses Lipid Peroxidation in Mitochondria

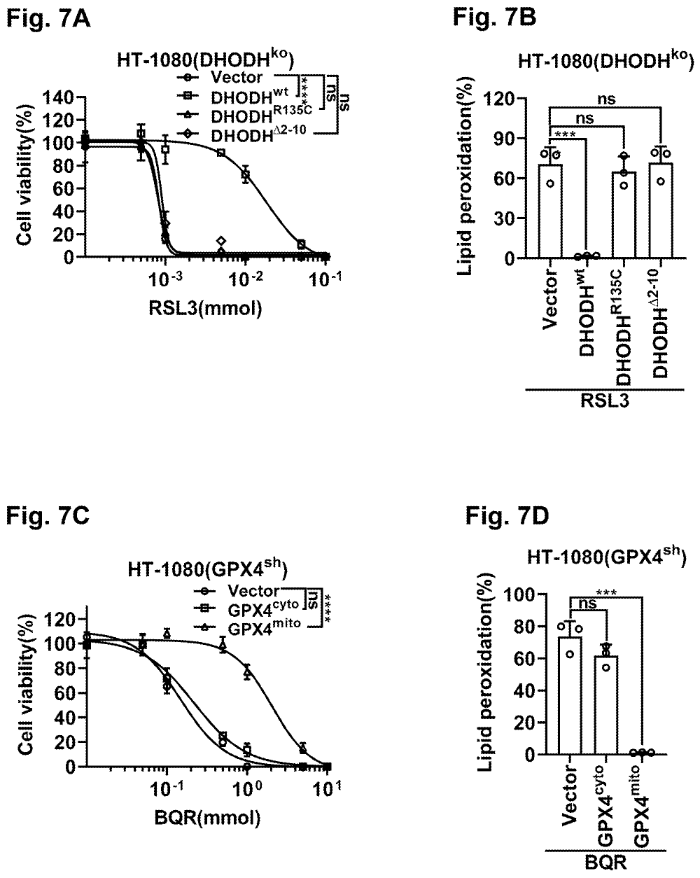

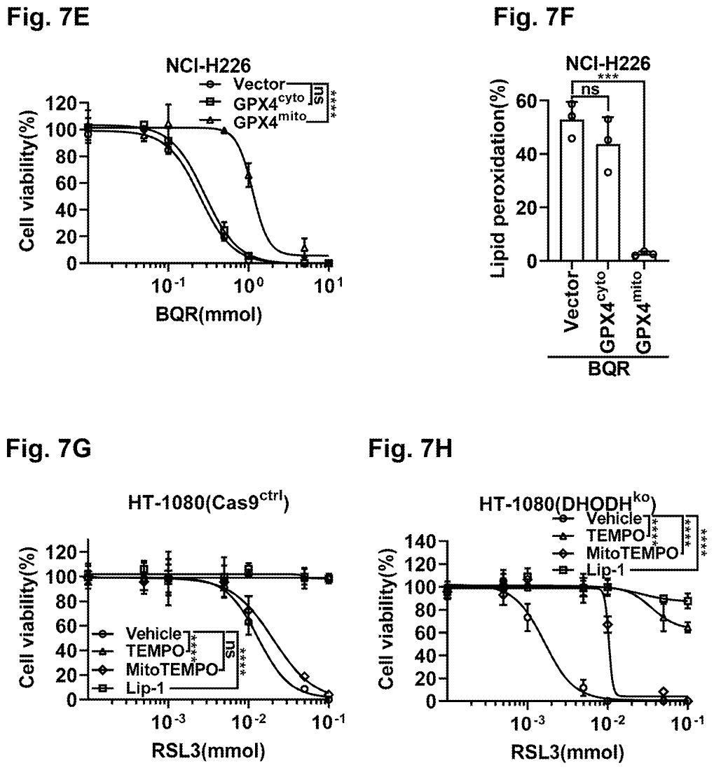

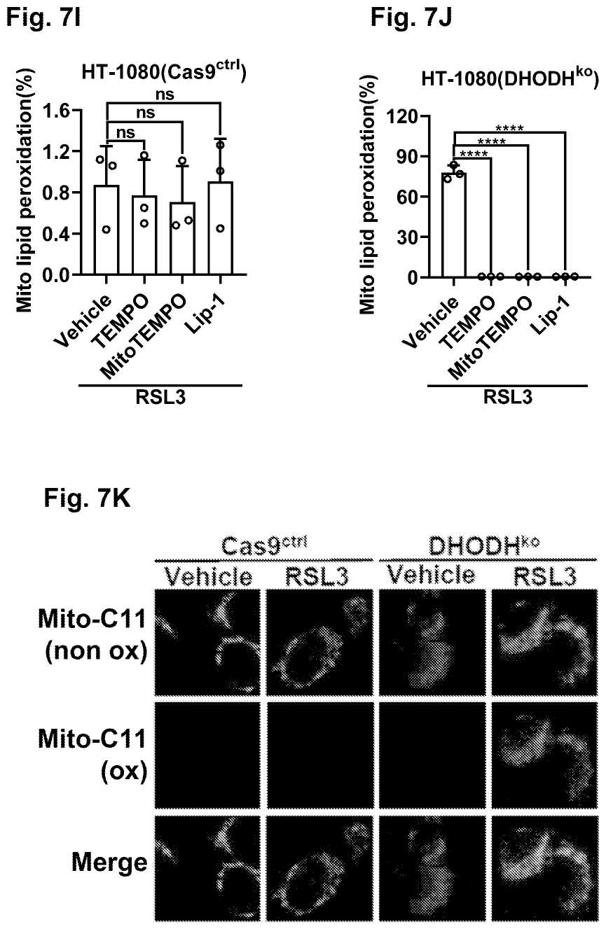

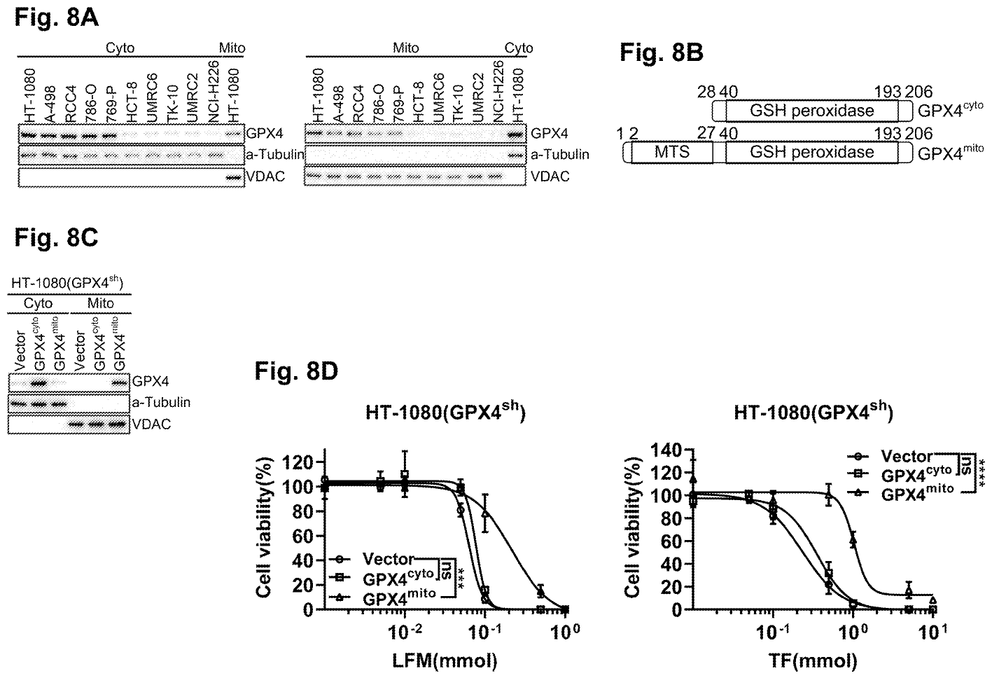

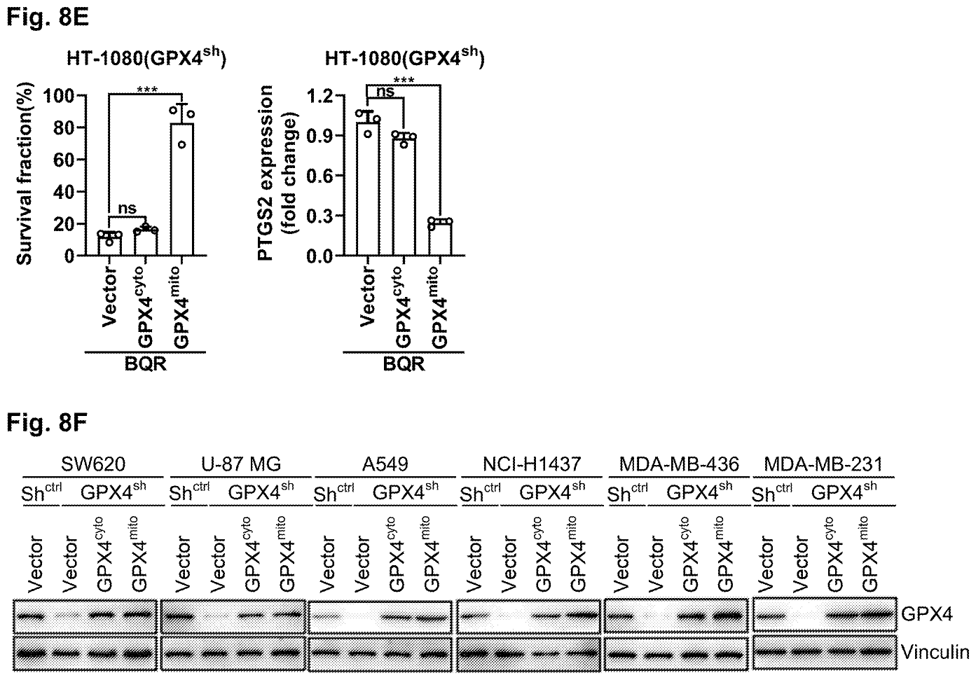

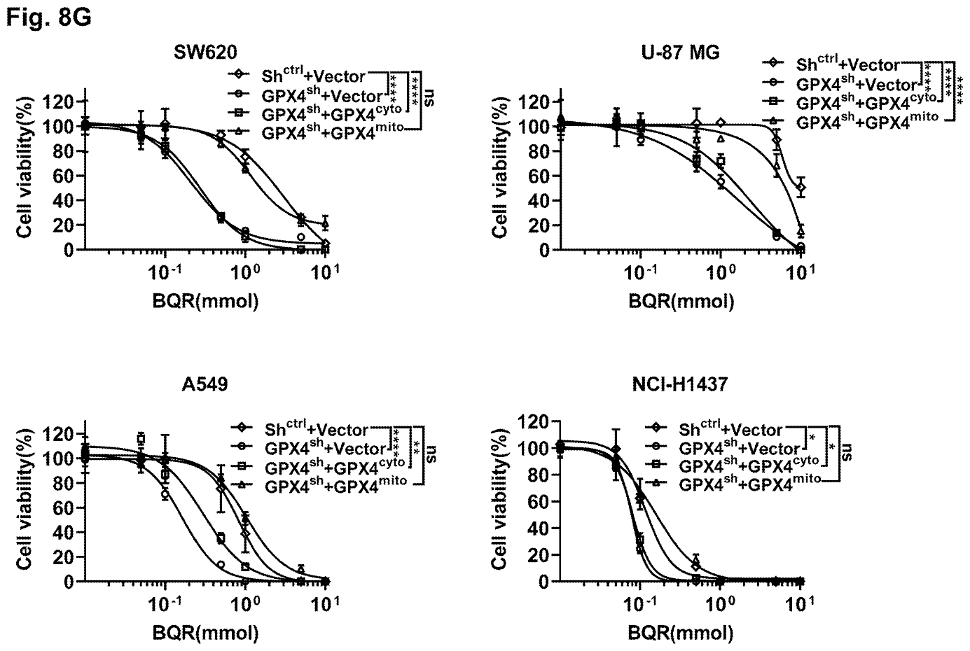

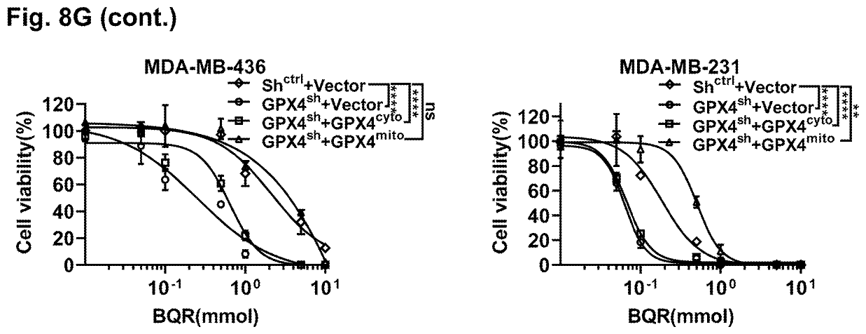

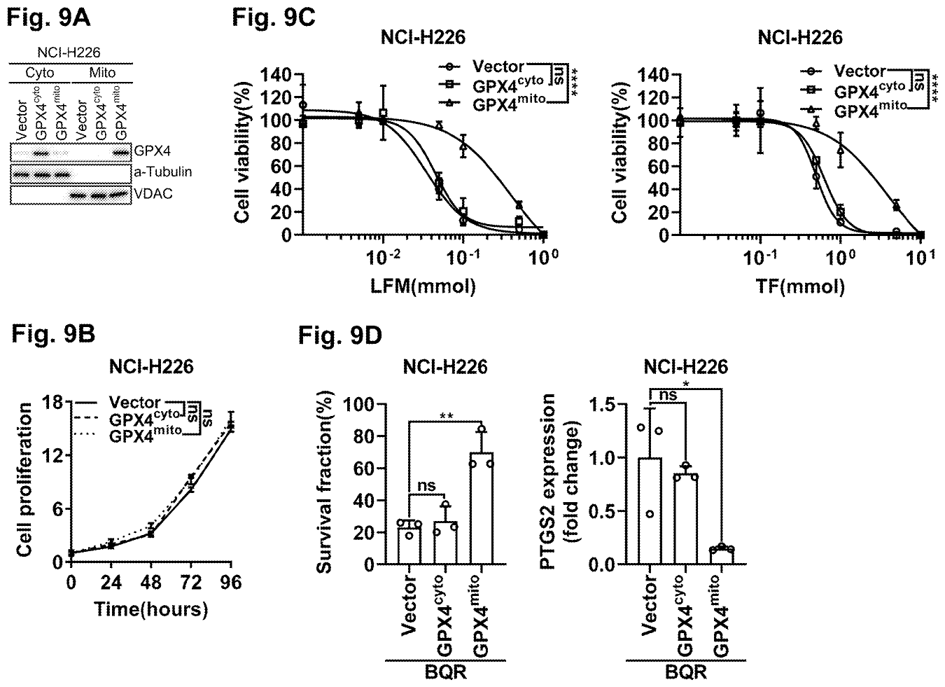

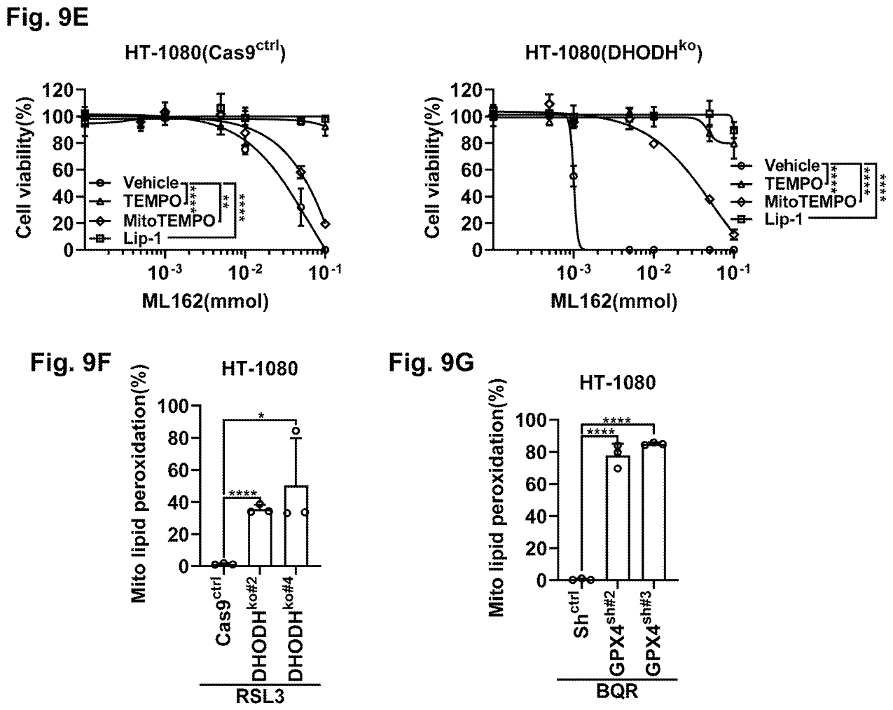

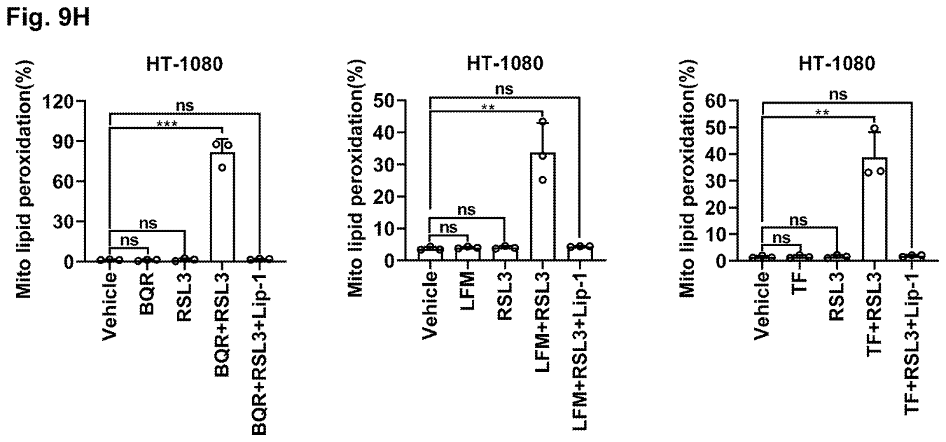

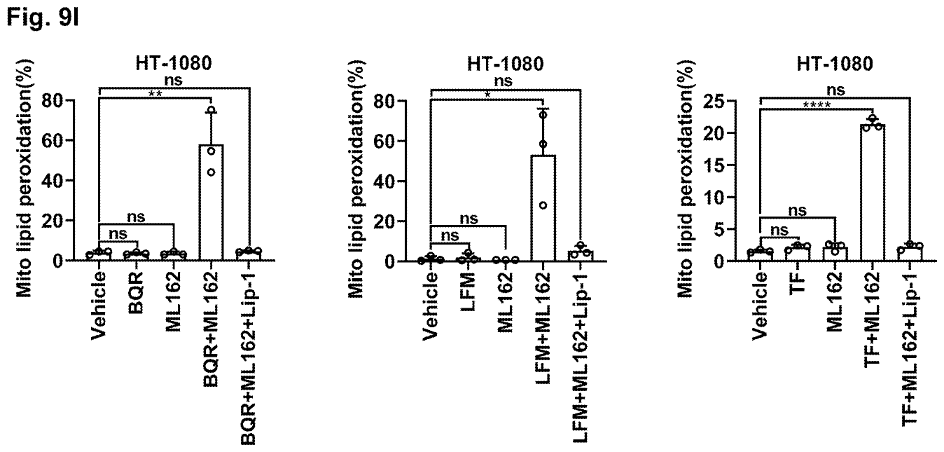

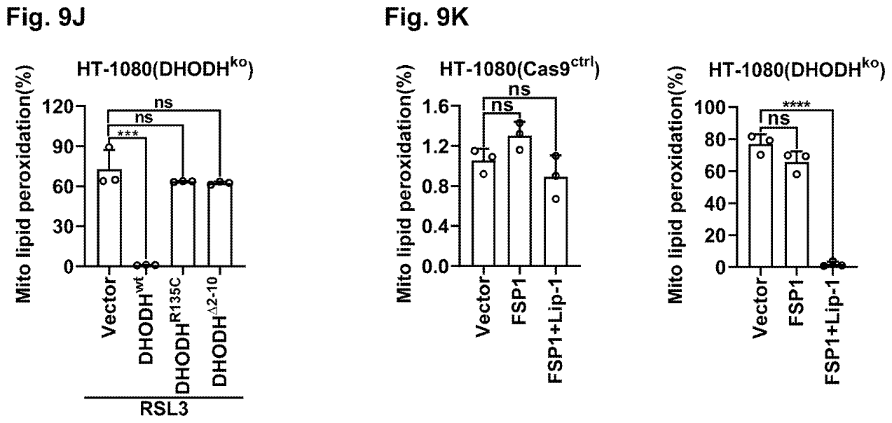

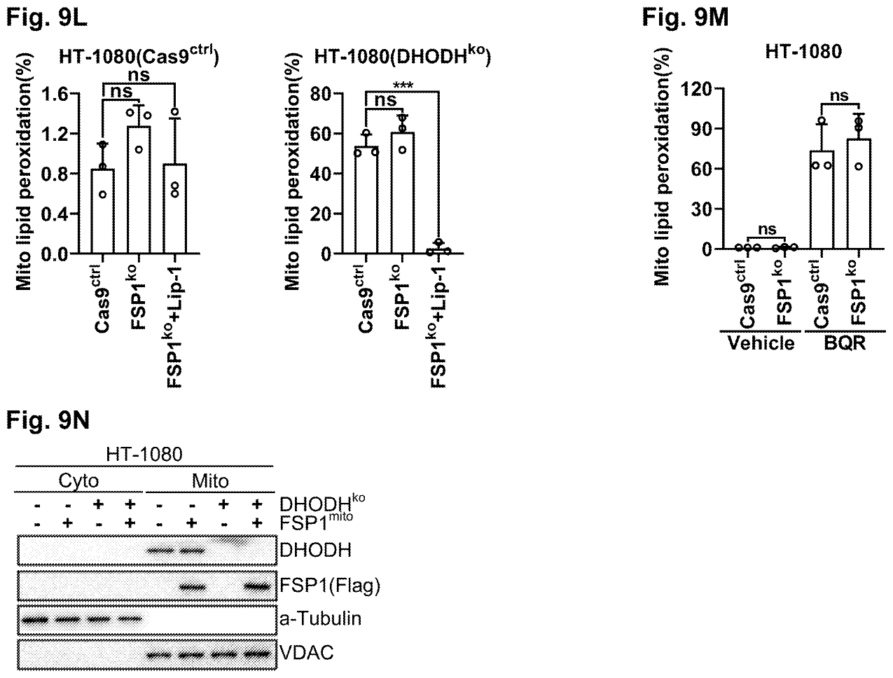

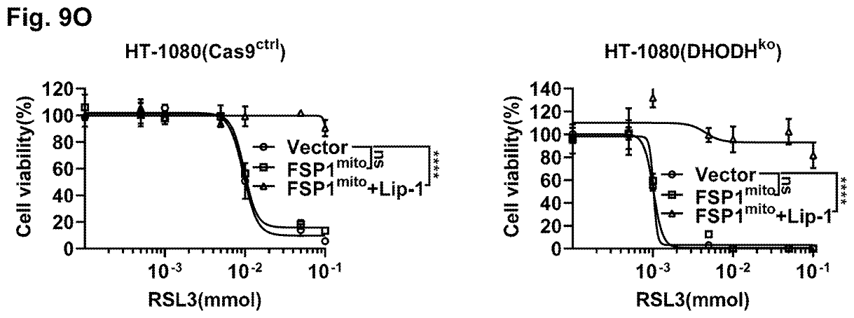

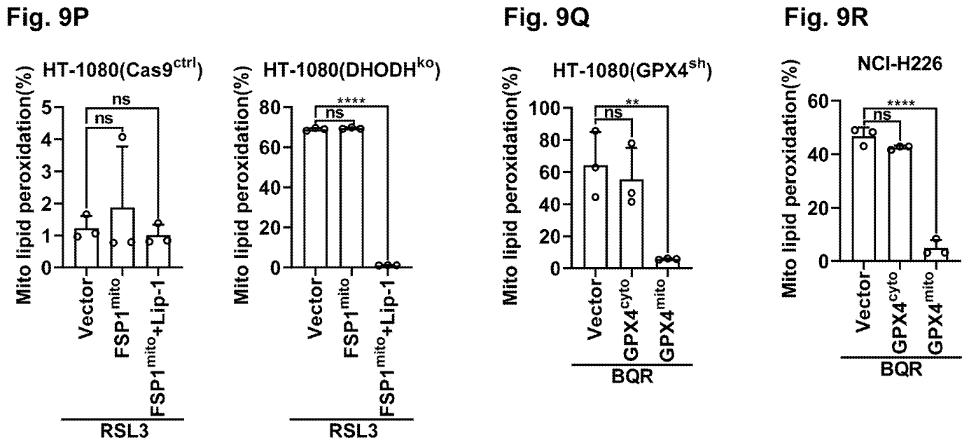

DHODH is an enzyme localized on the outer face of the mitochondrial inner membrane (Madak, J. T., Bankhead, A., 3rd, Cuthbertson, C. R., Showalter, H. D. & Neamati, N., Pharmacol Ther 195, 111-131 (2019)). It can be seen that restoration of DHODH WT, but not its catalytically inactive mutant (R135C) or a mutant defective in mitochondrial localization (Δ2-12), restored ferroptosis sensitivity to GPX4 inhibitors in DHODH KO HT-1080 cells (FIG. 7A, FIG. 7B, FIG. 6M, FIG. 6N, FIG. 6O, FIG. 6P, and FIG. 6Q). Mammalian cells encode several GPX4 isoforms with distinctive subcellular localization, including cytosol- and mitochondria-localized GPX4 (GPX4^{cyto} and GPX4^{mito})¹³. Fractionation analyses revealed that GPX4^{high} cancer cells generally exhibited high expression of both GPX4^{mito} and GPX4^{cyto} (FIG. 8A). Restoration of GPX4^{mito}, but not GPX4^{cyto}, in GPX4 knockdown HT-1080 cells rescued cellular sensitivity to DHODH inhibition (FIG. 7C, FIG. 7D, FIG. 8B, FIG. 8C, FIG. 8D, and FIG. 8E). This occurred in a variety of cell lines (FIG. 8F and FIG. 8G). Likewise, overexpression of GPX4^{mito}, but not GPX4^{cyto}, in NCI-H226 cells rendered these cells more resistant to DHODH inhibition and mitigated lipid peroxidation induced by DHODH inhibitors (FIG. 7E, FIG. 7F, FIG. 9A, FIG. 9B, FIG. 9C, and FIG. 9D). It can be seen that the RTA 2,2,6,6-tetramethylpiperidinyl-1-oxy (TEMPO), similar to liproxstatin-1, rescued GPX4 inhibition-induced ferroptosis in both DHODH WT and KO HT-1080 cells; in contrast, mitochondria-targeted TEMPO (mito-TEMPO) (Trnka, J., Blaikie, F. H., Smith, R. A. & Murphy, M. P., Free Radic Biol Med 44, 1406-1419 (2008)) provided substantial protection in DHODH KO but not in its WT counterparts (FIG. 7G, FIG. 7H, and FIG. 9E).

Consistent with a previous report (Friedmann Angeli, J. P., et al., Nat Cell Biol 16, 1180-1191 (2014)), staining of mito-BoDIPY (a mitochondria-targeted version of C11-BoDIPY to specifically detect mitochondrial lipid peroxidation (Prime, T. A., et al., Free Radic Biol Med 53, 544-553 (2012))) showed no mitochondrial lipid peroxidation upon GPX4 inhibition in HT-1080 cells (FIG. 7I); remarkably, GPX4 inhibitors induced potent mitochondrial lipid peroxidation in DHODH KO HT-1080 cells, which could be suppressed by TEMPO, mito-TEMPO, or liproxstatin-1 treatment (FIG. 7J). Confocal microscopy analysis of mito-BODIPY staining confirmed RSL3-induced mitochondrial lipid peroxidation in DHODH KO but not WT cells (FIG. 7K). Likewise, combined inactivation of GPX4 and DHODH, but not either single inactivation, massively induced mitochondrial lipid peroxidation (FIG. 9F, FIG. 9G, FIG. 9H, FIG. 9I, and FIG. 9J). FSP1 overexpression or deletion did not affect mitochondrial lipid peroxidation in either DHODH WT or KO cells (FIG. 9K, FIG. 9L, and FIG. 9M). Overexpression of mitochondria-localized FSP1 (FSP1^{mito}, by adding a mitochondrial targeting sequence to the N-terminus of FSP1) did not affect mitochondrial lipid peroxidation or provide any protection to RSL3-induced ferroptosis in HT-1080 cells or DHDOH KO counterparts (FIG. 9N, FIG. 9O, and FIG. 9P). These data confirm a critical role of FSP1's plasma membrane localization in mediating its anti-ferroptosis function. Further, targeting FSP1 to mitochondria alone is not sufficient to drive its presumed anti-ferroptosis function in mitochondria, likely because FSP1's function in ferroptosis regulation requires additional regulatory proteins that localize in cytosol or on the plasma membrane, and/or because the addition of a mitochondrial targeting sequence disrupts N-terminal myristoylation of FSP1, which is required for its function in suppressing ferroptosis (Bersuker, K., et al., Nature 575, 688-692 (2019); Doll, S., et al., Nature 575, 693-698 (2019)). Finally, it can be seen that restoration of GPX4^{mito}, but not GPX4^{cyto}, suppressed mitochondrial lipid peroxidation caused by DHODH inhibition in GPX4 knockdown HT-1080 cells or GPX4^{low} NCI-H226 cells (FIG. 9Q and FIG. 9R). Together, this data support a model wherein DHODH and mitochondria-localized GPX4 act concertedly to suppress mitochondrial lipid peroxidation and ferroptosis.

### DHODH Suppresses Ferroptosis Through Reducing CoQ to CoQH2 in Mitochondria

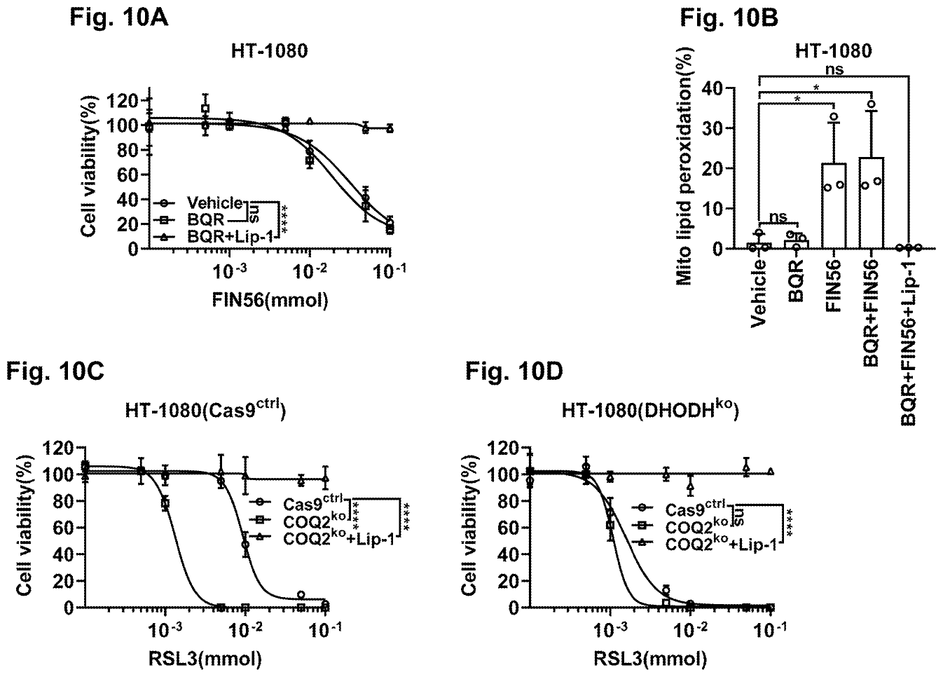

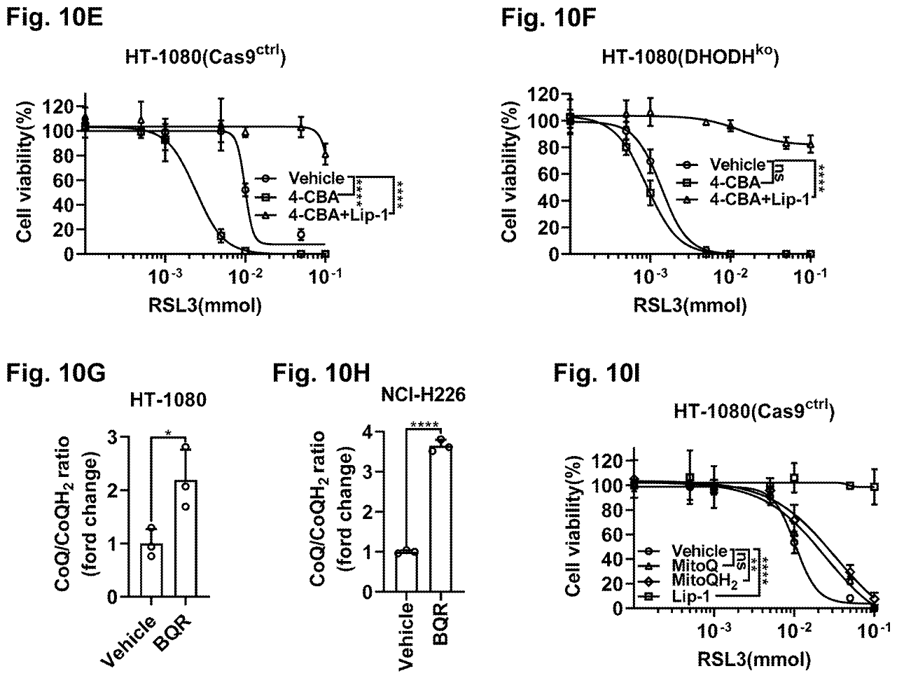

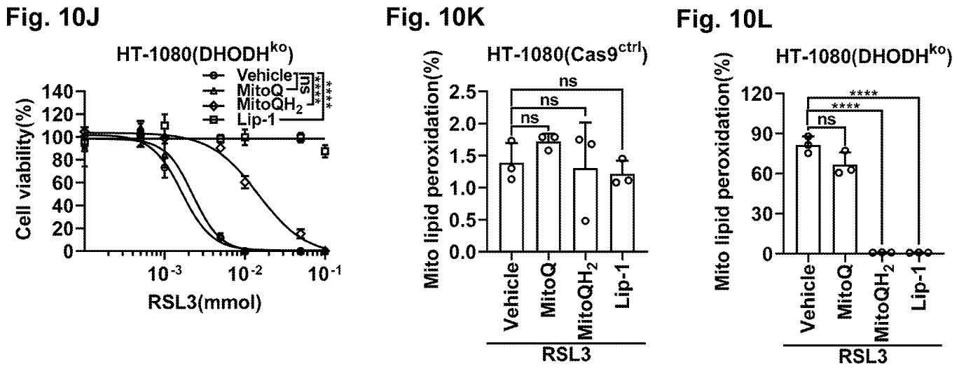

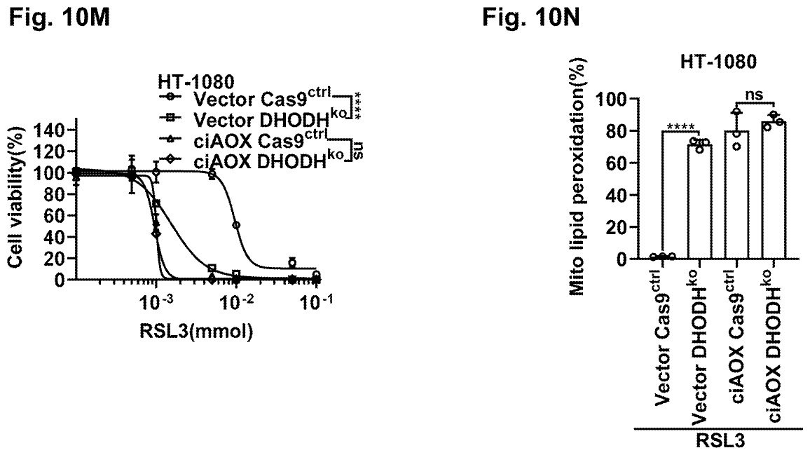

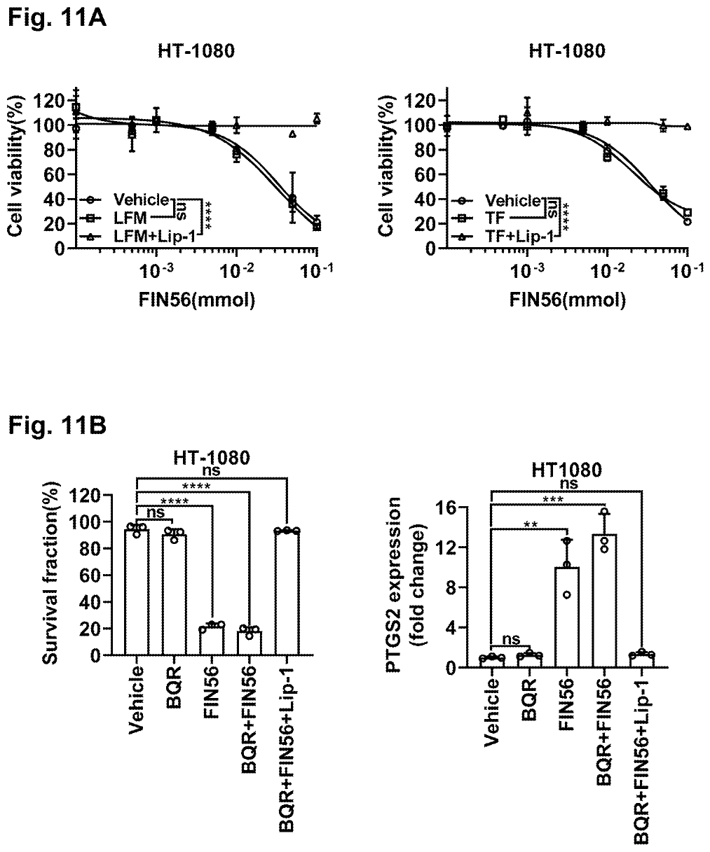

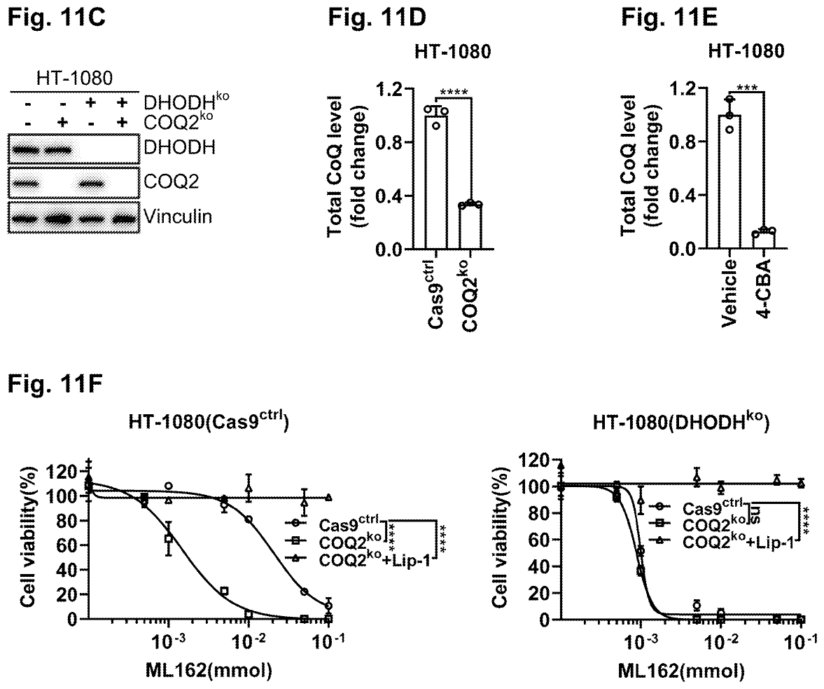

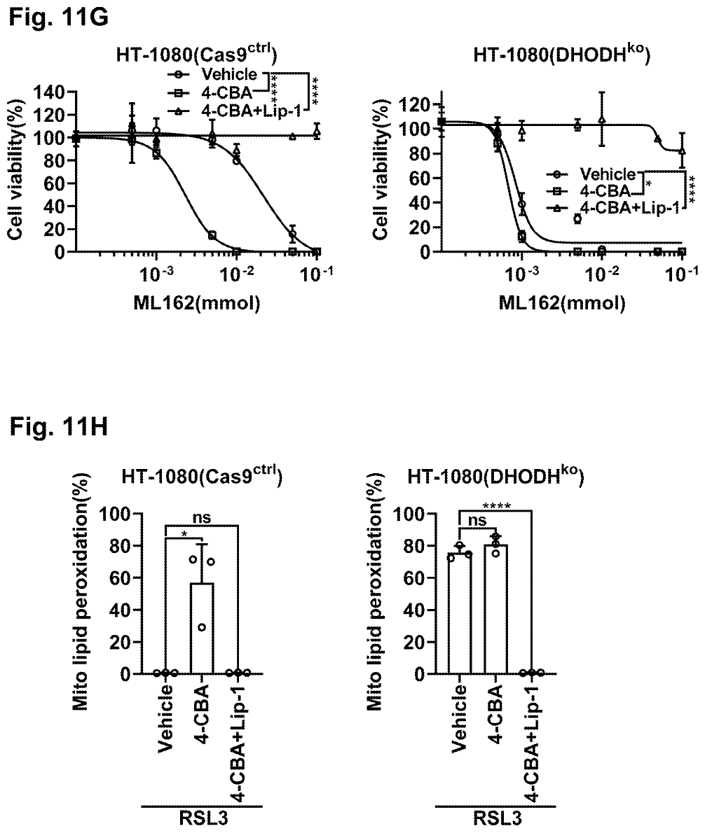

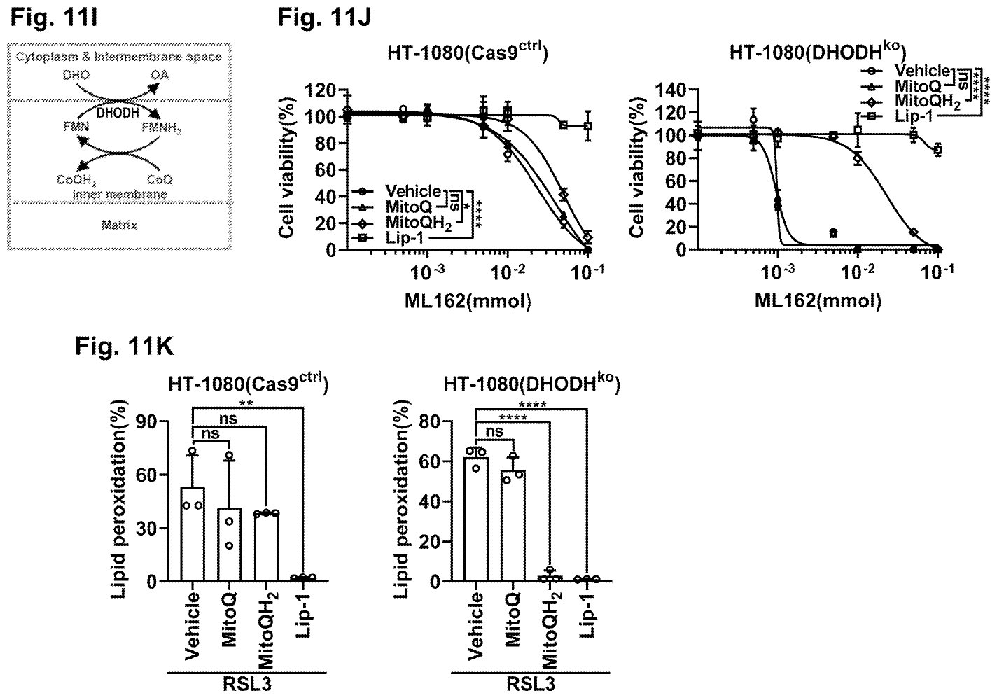

Next, the mechanisms by which DHODH suppresses mitochondrial lipid peroxidation and ferroptosis were examined. It can be seen that DHODH inhibition sensitized HT-1080 cells to class 1 or 2 FINs (FIG. 1L, FIG. 1M, FIG. 2C, FIG. 2D, FIG. 2E, FIG. 2F, and FIG. 2G), but surprisingly, not to the class 3 FIN FIN56, which acts by depleting both GPX4 protein and CoQ (Shimada, K., et al., Nature chemical biology 12, 497-503 (2016)) (FIG. 10A, FIG. 11A, and FIG. 11B), suggesting that DHODH regulates ferroptosis in a CoQ-dependent manner. Unlike RSL3, FIN56 treatment induced substantial mitochondrial lipid peroxidation, and DHODH deletion did not further increase mitochondrial lipid peroxidation under FIN56 treatment (FIG. 10B). Deletion of COQ2, a key enzyme involved in CoQ biosynthesis, or blocking CoQ biosynthesis by 4-chlorobenzoic acid (4-CBA) treatment significantly decreased total CoQ levels (FIG. 11C, FIG. 11D, and FIG. 11E), and promoted ferroptosis much more dramatically in HT-1080 cells than in their DHODH KO counterparts (FIG. 10C, FIG. 10D, FIG. 10E, FIG. 10F, FIG. 11F, FIG. 11G, and FIG. 11H). These data together indicate that DHODH acts in the same signaling axis with CoQ biosynthesis pathways to suppress mitochondrial lipid peroxidation and ferroptosis.

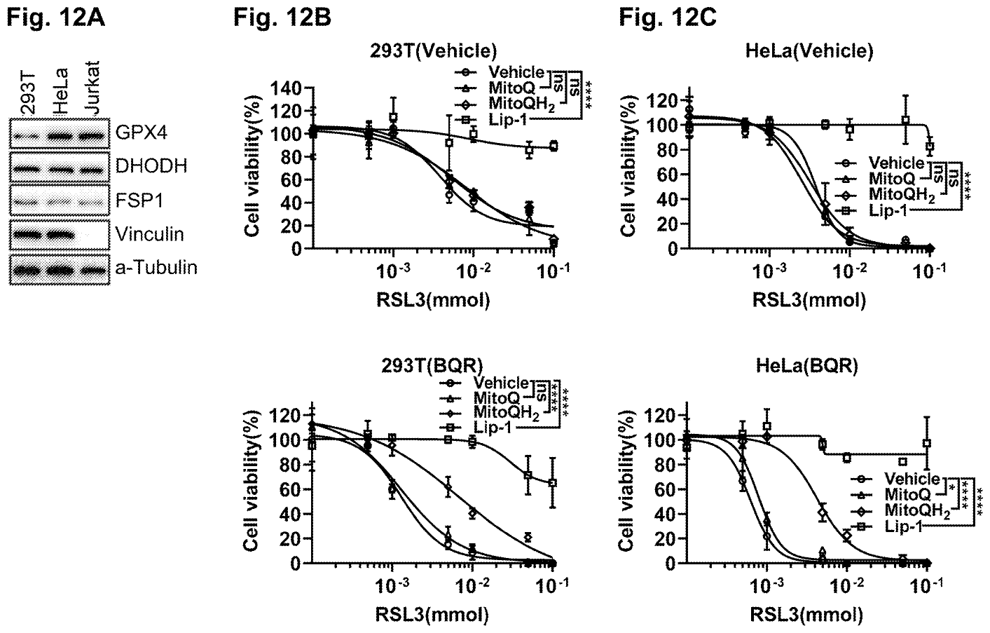

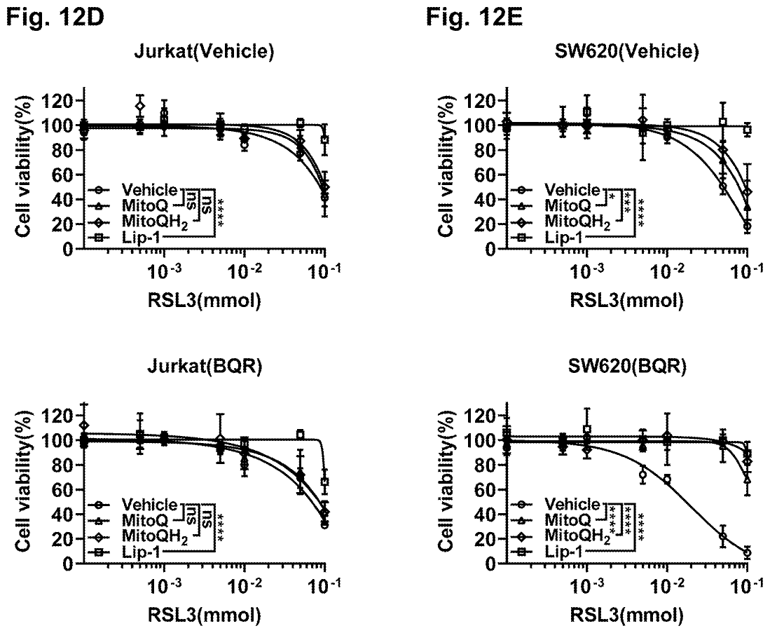

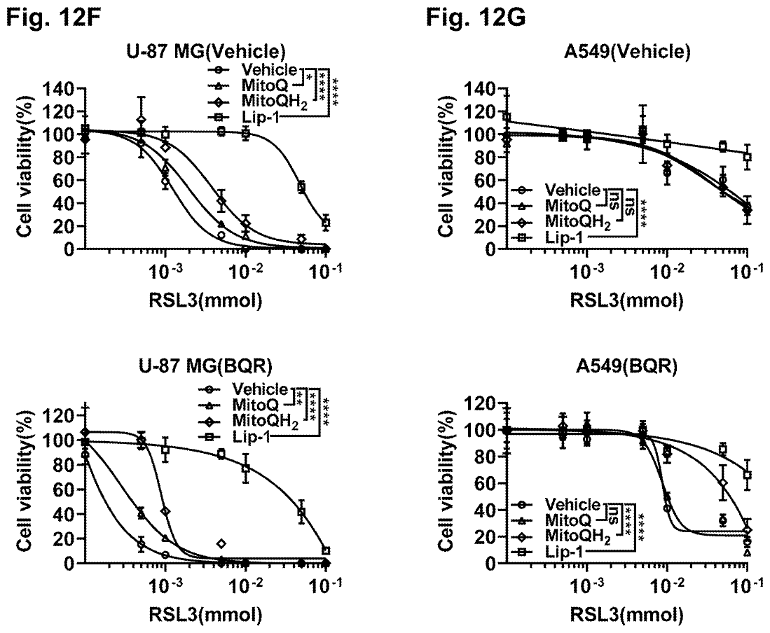

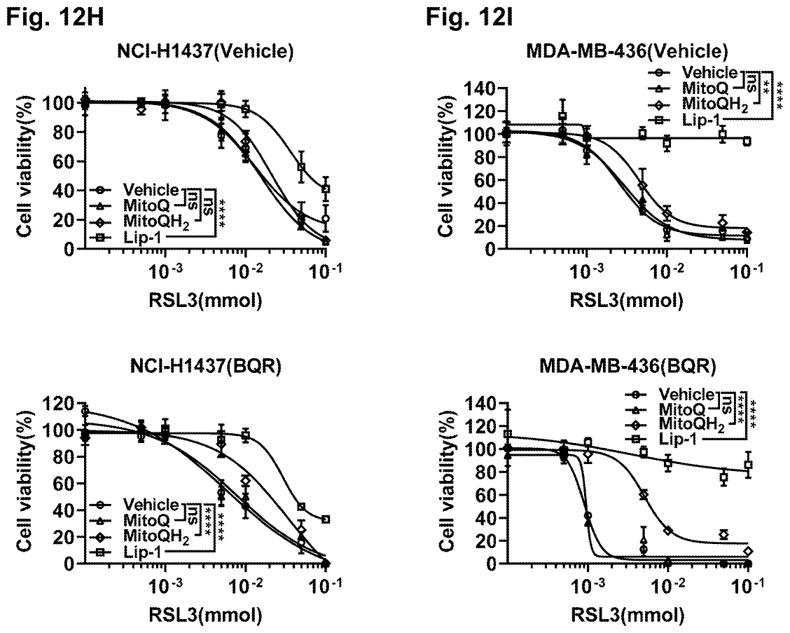

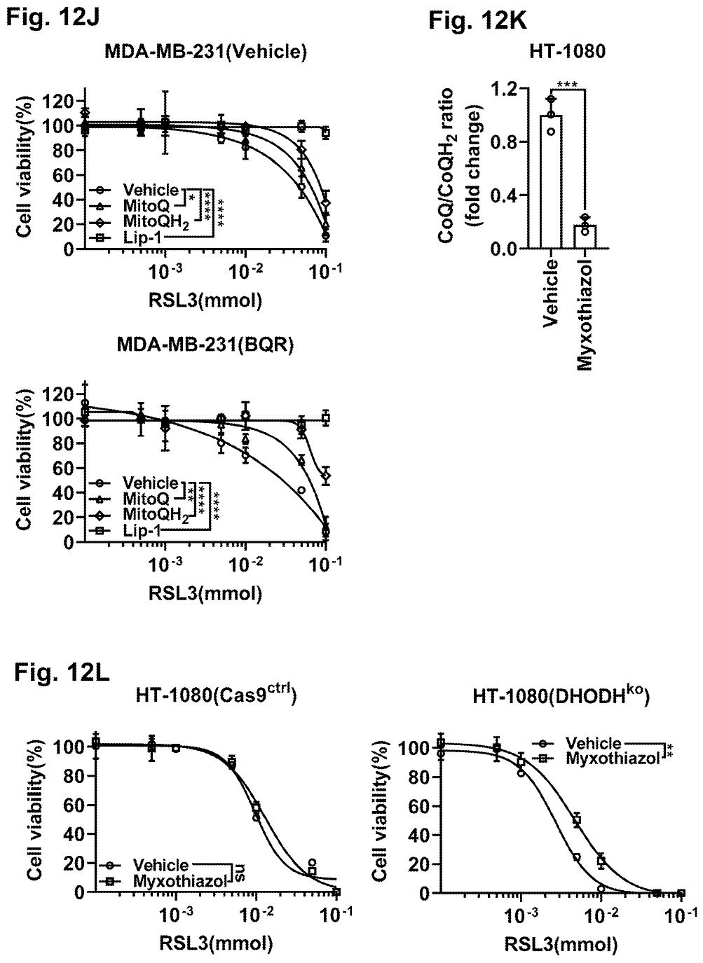

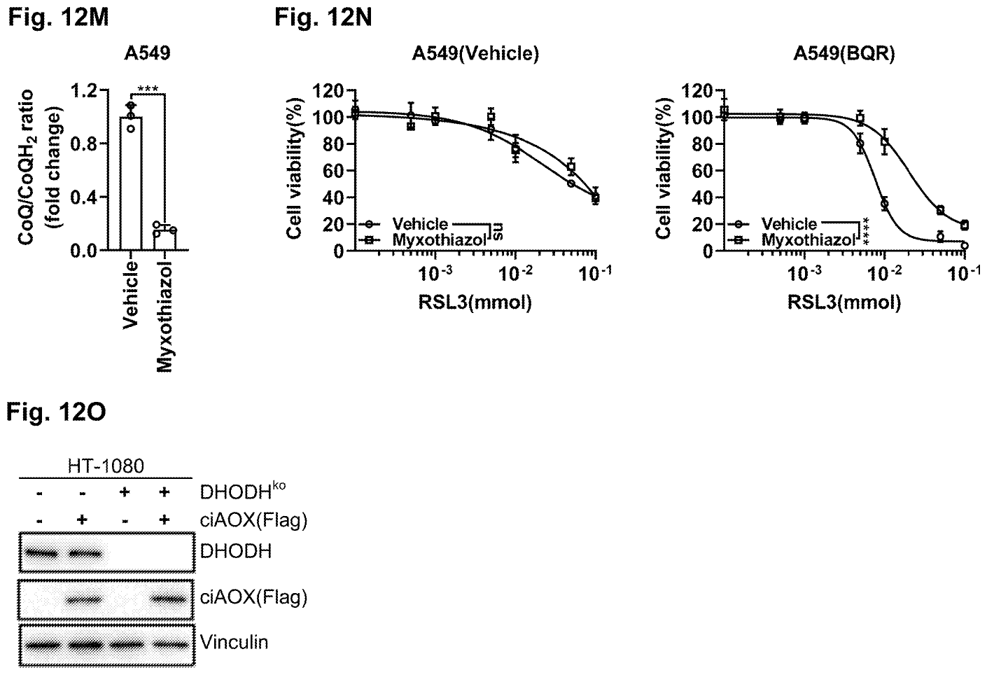

DHODH couples the oxidation of DHO to OA to the reduction of CoQ to CoQH₂ in the mitochondrial inner membrane (Madak, J. T., Bankhead, A., 3rd, Cuthbertson, C. R., Showalter, H. D. & Neamati, N., Pharmacol Ther 195, 111-131 (2019)) (FIG. 11I). It can be seen that DHODH inhibition significantly increased CoQ/CoQH2 ratio (FIG. 10G and FIG. 10H). Supplementation of mitoQ and mitoQH₂ (mitochondria-targeted analogs of CoQ and CoQH₂, respectively) provided limited protection against RSL3- or ML162-induced ferroptosis in HT-1080 cells (FIG. 10I and FIG. 11J) (Friedmann Angeli, J. P., et al., Nat Cell Biol 16, 1180-1191 (2014)); in contrast, mitoQH₂, but not mitoQ, significantly protected against RSL3- or ML162-induced mitochondrial lipid peroxidation and ferroptosis in DHODH KO cells (FIG. 10J, FIG. 10K, FIG. 10L, FIG. 11J, and FIG. 11K). These results indicate that (i) mitoQH₂ mainly inhibits ferroptosis in cells with extensive mitochondrial lipid peroxidation (such as DHODH KO cells with GPX4 inactivation), and (ii) DHODH is required for reducing mitoQ to mitoQH₂ to suppress ferroptosis. These observations are also seen in most of the other cell lines tested (FIG. 12A, FIG. 12B, FIG. 12C, FIG. 12D, FIG. 12E, FIG. 12F, FIG. 12G, FIG. 12H, FIG. 12I, and FIG. 12J).

Electron transport chain (ETC) complex III converts CoQH₂ back to CoQ. Treatment with complex III inhibitor myxothiazol significantly decreased CoQ/CoQH₂ ratio, and similar to mitoQH₂ treatment, did not affect ferroptosis sensitivity in HT-1080 cells yet protected against RSL3-induced ferroptosis in DHODH KO counterparts, albeit with a more moderate protective effect than mitoQH₂ (FIG. 10J, FIG. 12K, FIG. 12L, FIG. 12M, and FIG. 12N). This is likely because upon GPX4 inactivation, much of the CoQH₂ pool is consumed by quenching mitochondrial lipid peroxides, leaving less CoQH₂ available to feed complex III. Finally, overexpression of Ciona intestinalis AOX, which oxidizes mitochondrial CoQH₂ to CoQ without pumping protons (Hakkaart, G. A., Dassa, E. P., Jacobs, H. T. & Rustin, P., EMBO Rep 7, 341-345 (2006)), had a dramatic sensitizing effect on RSL3-induced mitochondrial lipid peroxidation and ferroptosis in HT-1080 cells; importantly, DHODH deletion did not provide additional ferroptosis sensitizing effect in AOX-overexpressing cells (FIG. 10M, FIG. 10N, and FIG. 12O). Without being bound by theory, the data support a model that DHODH inhibits ferroptosis through reducing CoQ to CoQH₂ in mitochondria.

### DHODH Inhibitors Suppress GPX4^{low} Tumor Growth Through Inducing Ferroptosis In Vivo

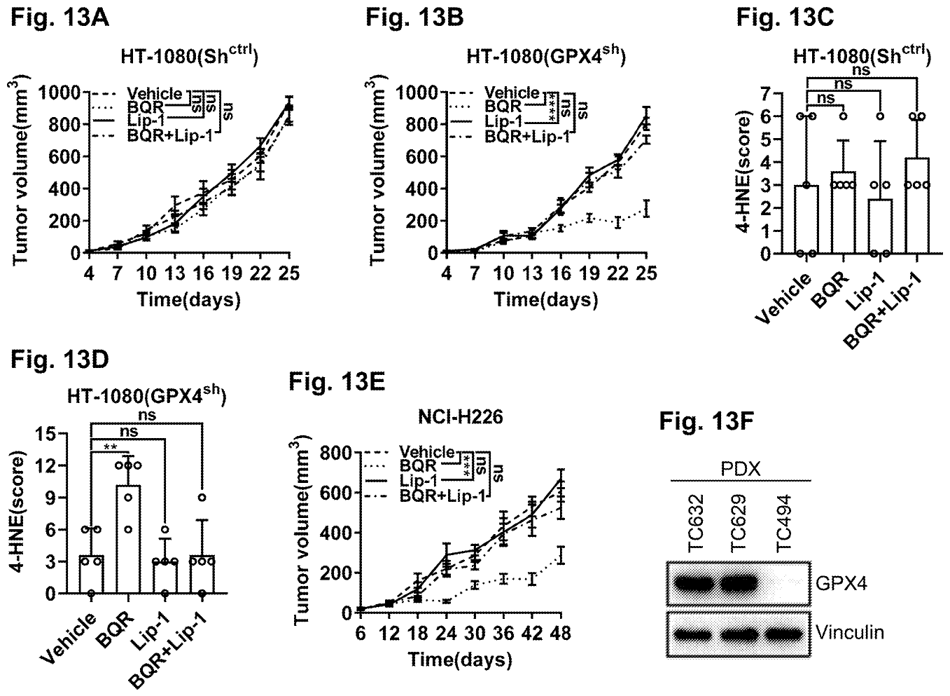

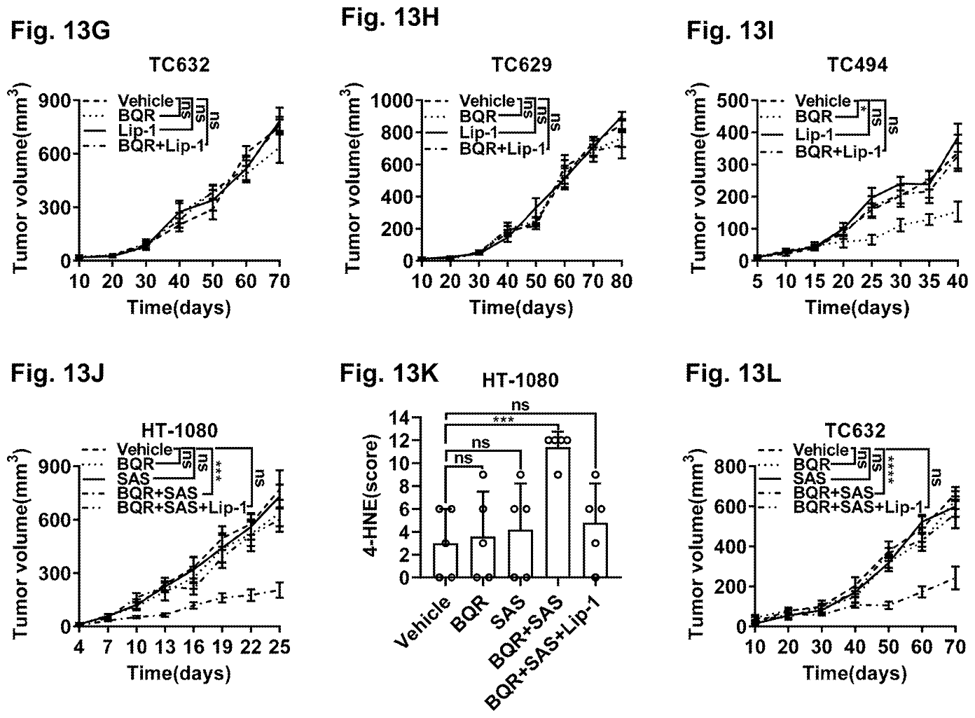

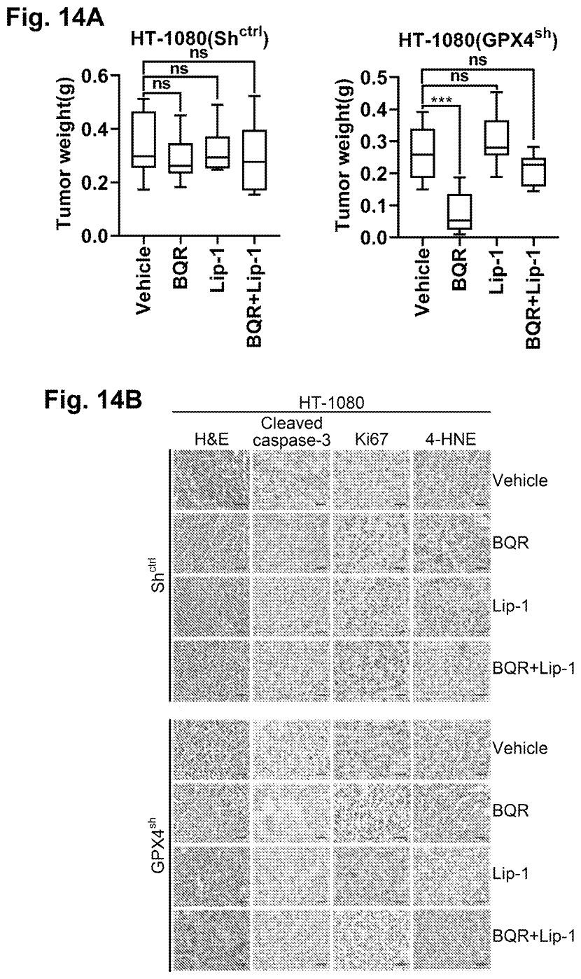

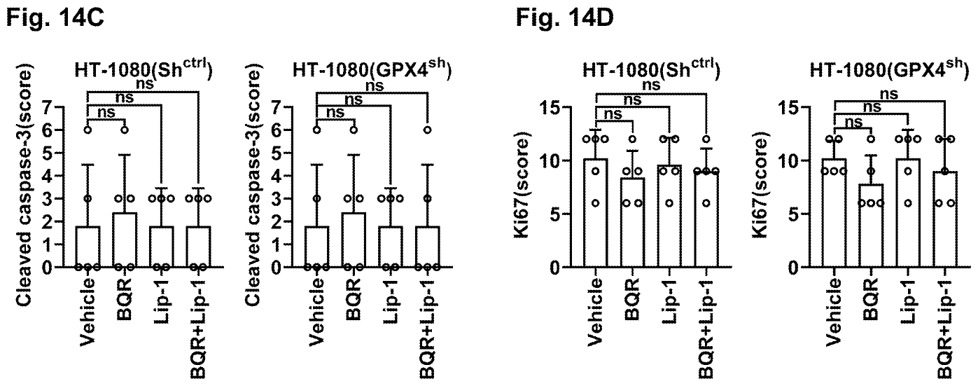

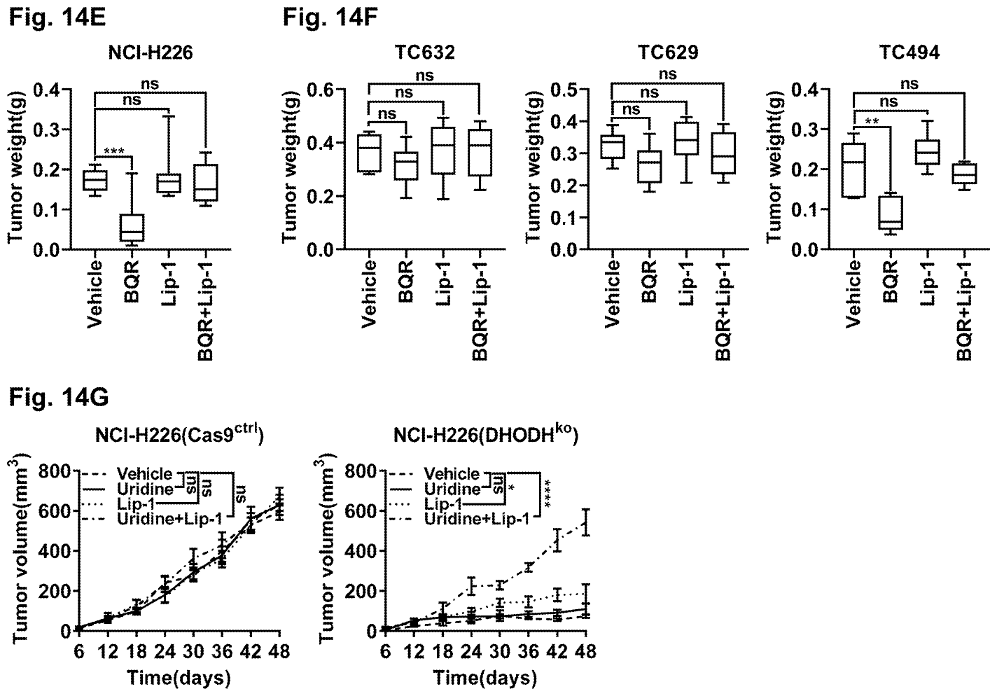

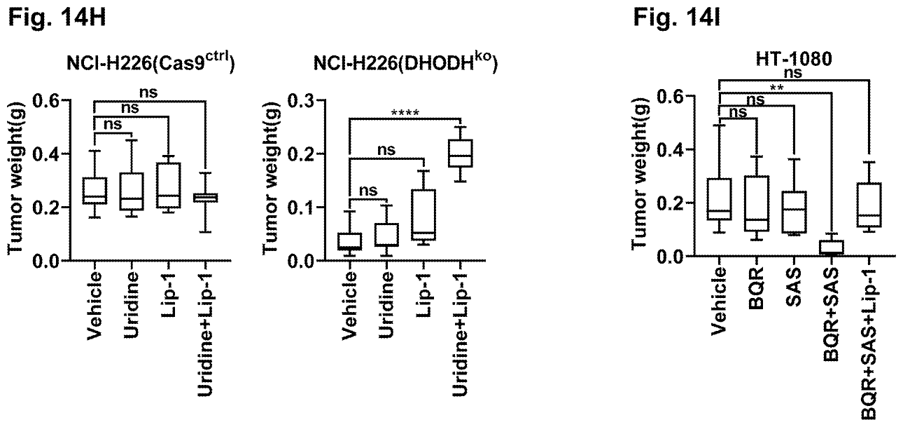

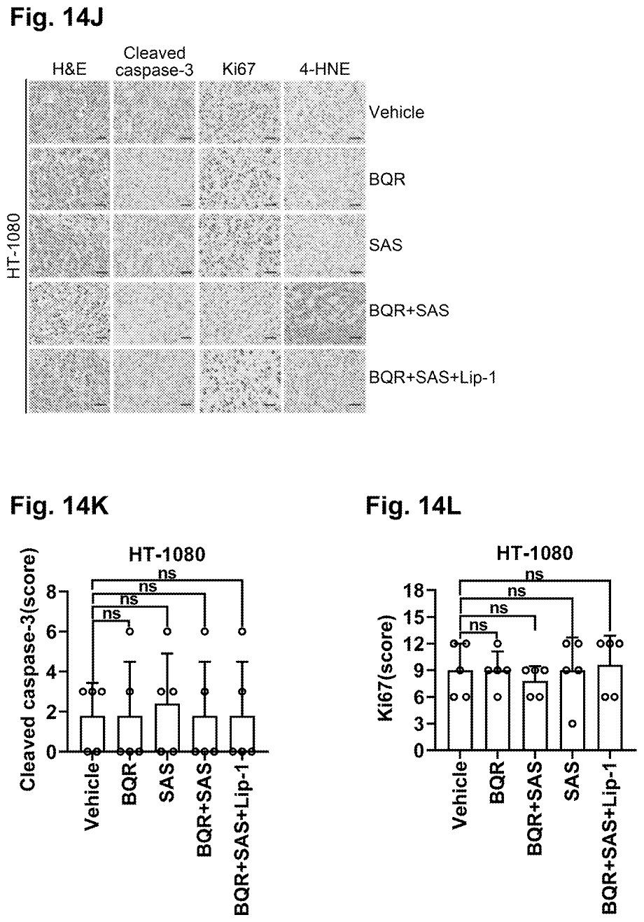

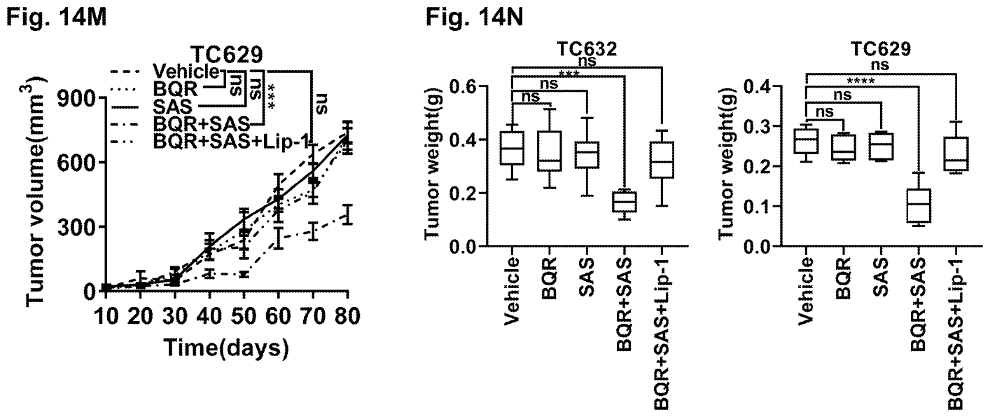

Without being bound by theory, as the data suggested that GPX4^{low} cancer cells are particularly sensitive to DHODH inhibition-induced ferroptosis, the therapeutic potential of DHODH inhibitors in treating GPX4^{low} tumors in vivo was then investigated. Brequinar, a potent and specific DHODH inhibitor (Peters, G. J., et al., Cancer Res 50, 4644-4649 (1990); Natale, R., et al., Ann Oncol 3, 659-660 (1992); Urba, S., et al., Cancer Chemother Pharmacol 31, 167-169 (1992); Maroun, J., et al., Cancer Chemother Pharmacol 32, 64-66 (1993)), was used in these studies. While brequinar treatment or GPX4 knockdown alone did not affect the growth of HT-1080 xenograft tumors, GPX4 knockdown significantly sensitized HT-1080 xenograft tumors to DHODH inhibition; and, treatment with liproxstatin-1 largely restored the growth of GPX4-knockdown tumors under brequinar treatment (FIG. 13A, FIG. 13B, and FIG. 14A). Further analysis revealed that brequinar treatment did not affect cleaved caspase-3 or Ki67 staining in both control and GPX4-knockdown xenograft samples (FIG. 14B, FIG. 14C, and FIG. 14D); however, brequinar treatment dramatically increased 4-HNE staining, a lipid peroxidation marker (Lei, G., et al., Cell research 30, 146-162 (2020)), in GPX4-knockdown tumors, but not in control tumors (FIG. 13C, FIG. 13D, and FIG. 14B). Similarly, brequinar significantly suppressed the tumor growth of GPX4^{low} NCI-H226 xenografts or GPX4^{low} patient-derived xenografts (PDXs), but not that of GPX4^{high} PDXs, and the suppressed tumor growth in NCI-H226 xenografts or GPX4^{low} PDXs could be restored by liproxstatin-1 treatment (FIG. 13E, FIG. 13F, FIG. 13G, FIG. 13H, FIG. 13I, FIG. 14E, and FIG. 14F). In addition, DHODH ablation markedly suppressed NCI-H226 xenograft tumor growth; yet, combined treatment with liproxstatin-1 and uridine, but not either treatment alone, almost completely restored the growth of DHODH KO tumors (FIG. 14G and FIG. 14H), which is consistent with in vitro data (FIG. 4E).

Finally, it can be seen that combined treatment with brequinar and sulfasalazine, a class 1 FIN that inhibits SLC7A11 (Gout, P. W., Buckley, A. R., Simms, C. R. & Bruchovsky, N., Leukemia 15, 1633-1640 (2001)), synergistically induced lipid peroxidation and suppressed HT-1080 xenograft tumor growth, and the suppressed tumor growth could be largely restored by liproxstatin-1 treatment (FIG. 13J, FIG. 13K, FIG. 14I, FIG. 14J, FIG. 14K, and FIG. 14L). Similar observations were seen in GPX4^{high} PDXs (FIG. 13L, FIG. 14M, and FIG. 14N). In all these animal studies, drug treatment did not significantly affect animal weights (FIG. 14O), suggesting that the treatment was well-tolerated in vivo.

### Discussion

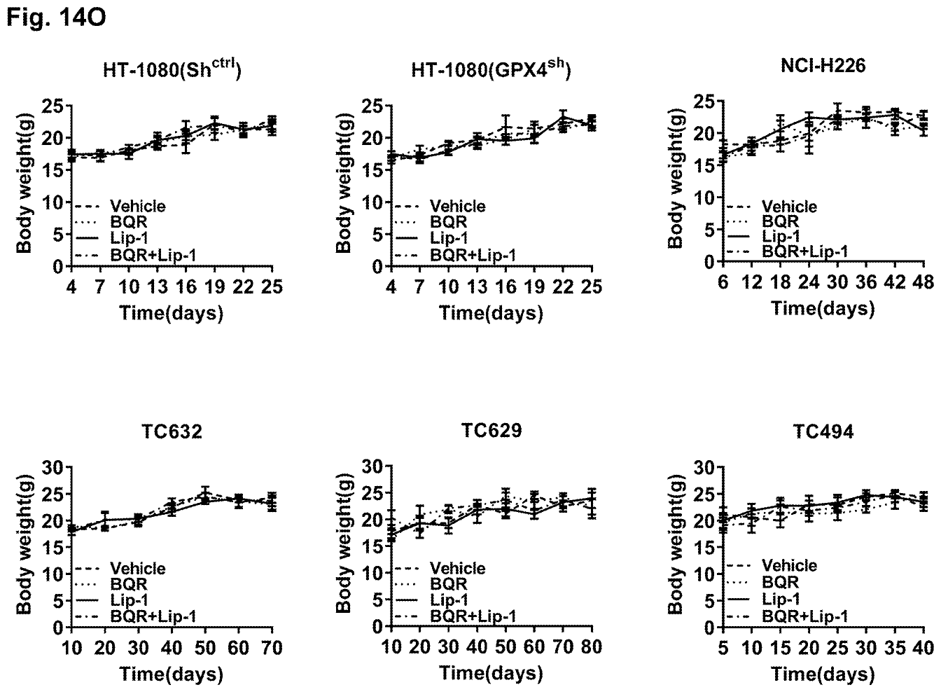

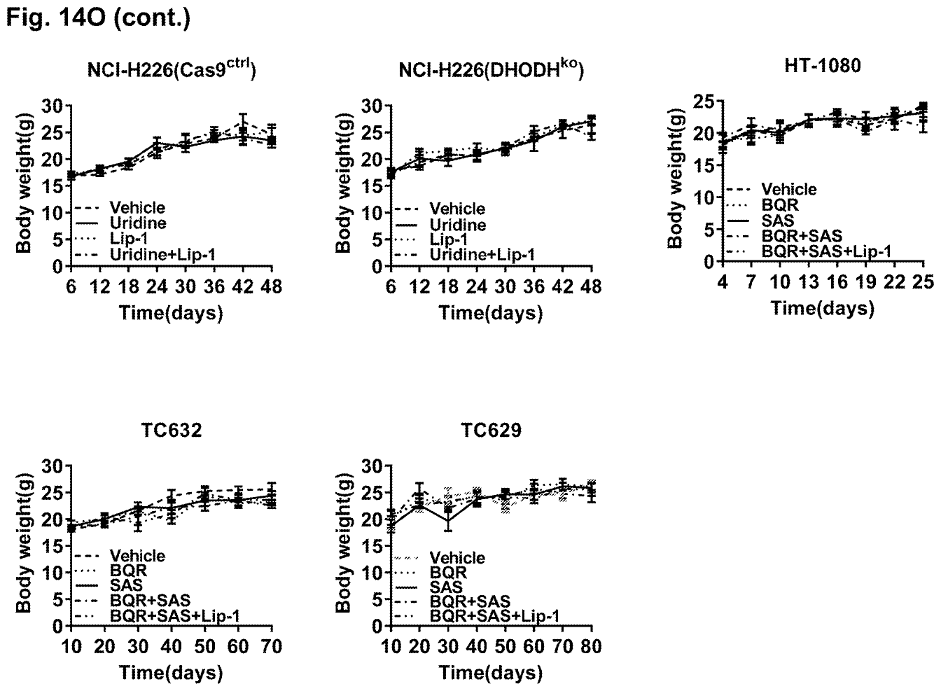

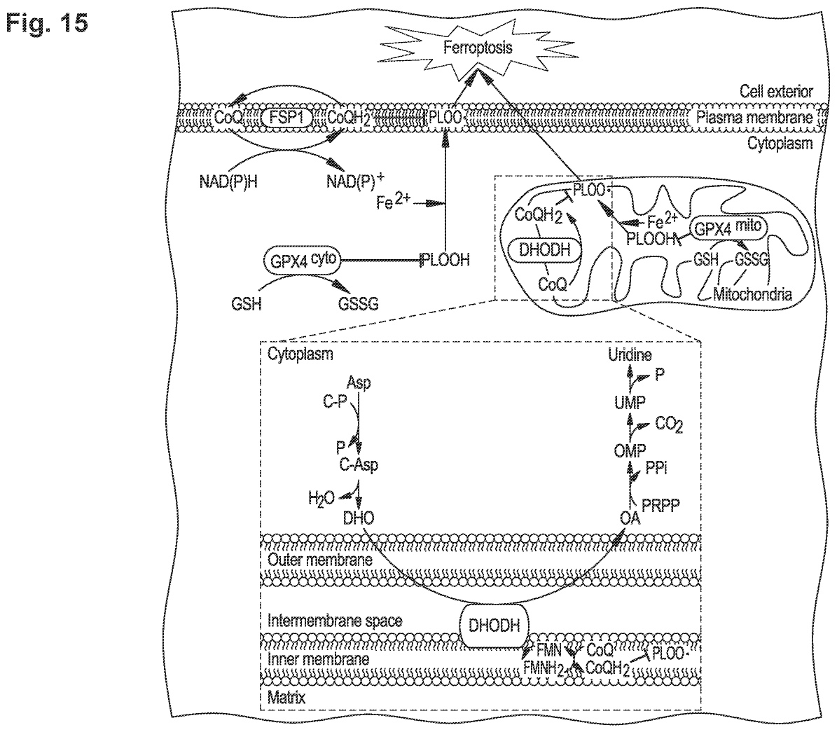

Without being bound by theory, there exist at least three cellular protective systems against ferroptosis with distinctive subcellular localization (FIG. 15): GPX4 in the cytosol and mitochondria, FSP1 on the plasma membrane, and DHODH in mitochondria. GPX4 constitutes a powerful defense mechanism against ferroptosis, related at least partly to GPX4's versatile localization in cells; therefore, GPX4 likely can detoxify lipid peroxides generated in all or most cellular membranes. Compartmentalization of ferroptosis defense mechanisms also plays a key role and provides a broad conceptual framework for further understanding ferroptosis regulation in different subcellular compartments. DHODH and mitochondrial GPX4 also constitute two major defense arms to detoxify mitochondrial lipid peroxides; consequently, disabling one arm forces cells to be more dependent on the other, and disabling both arms can trigger ferroptosis mainly induced by mitochondrial lipid peroxidation. Although DHODH was not identified from previous CRISPR screens (Zou, Y., et al., Nat Commun 10, 1617 (2019); Soula, M., et al., Nat Chem Biol 16, 1351-1360 (2020)), it is an essential gene. Thus, uridine supplementation is required for maintaining basal cell proliferation and therefore revealing ferroptosis phenotypes in DHODH KO cells.

While DHODH inhibitors, such as brequinar, have been tested in oncology clinical trials, which showed that brequinar is well-tolerated in patients but lacks broad efficacy against solid tumors (Peters, G. J., et al., Cancer Res 50, 4644-4649 (1990); Natale, R., et al., Ann Oncol 3, 659-660 (1992); Urba, S., et al., Cancer Chemother Pharmacol 31, 167-169 (1992); Maroun, J., et al., Cancer Chemother Pharmacol 32, 64-66 (1993)), there is a critical need to identify the specific context for therapeutic targeting of DHODH and/or rational drug combination strategies. The data described herein show that an array of GPX4^{low} solid tumors can be effectively targeted by DHODH inhibitors to reduce tumor burden, and that a combination of DHODH inhibitors with ferroptosis inducers, such as sulfasalazine, can be used to treat GPX4^{high} solid tumors. In addition, DHODH inhibitors can be combined with other standard-of-cares in cancer therapy that can induce ferroptosis, such as radiotherapy and immunotherapy (Lei, G., et al., Cell research 30, 146-162 (2020); Wang, W., et al., Nature 569, 270-274 (2019); Lang, X., et al., Cancer Discov (2019); Ye, L. F., et al., ACS Chem Biol 15, 469-484 (2020)).

### REFERENCES

1. Dixon, S. J. et al. Ferroptosis: an iron-dependent form of nonapoptotic cell death. Cell 149, 1060-1072 (2012).
2. Stockwell, B. R. et al. Ferroptosis: A Regulated Cell Death Nexus Linking Metabolism, Redox Biology, and Disease. Cell 171, 273-285 (2017).
3. Jiang, L. et al. Ferroptosis as a p53-mediated activity during tumour suppression. Nature 520, 57-62 (2015).
4. Zhang, Y. et al. BAP1 links metabolic regulation of ferroptosis to tumour suppression. Nat Cell Biol 20, 1181-1192 (2018).
5. Yang, W. S. et al. Regulation of ferroptotic cancer cell death by GPX4. Cell 156, 317-331 (2014).
6. Friedmann Angeli, J. P. et al. Inactivation of the ferroptosis regulator Gpx4 triggers acute renal failure in mice. Nat Cell Biol 16, 1180-1191 (2014).
7. Bersuker, K. et al. The CoQ oxidoreductase FSP1 acts parallel to GPX4 to inhibit ferroptosis. Nature 575, 688-692 (2019).
8. Doll, S. et al. FSP1 is a glutathione-independent ferroptosis suppressor. Nature 575, 693-698 (2019).
9. Koppula, P., Zhuang, L. & Gan, B. Cystine transporter SLC7A11/xCT in cancer: ferroptosis, nutrient dependency, and cancer therapy. Protein Cell (2020).
10. Doll, S. et al. ACSL4 dictates ferroptosis sensitivity by shaping cellular lipid composition. Nature chemical biology 13, 91-98 (2017).
11. Basu, A. et al. An interactive resource to identify cancer genetic and lineage dependencies targeted by small molecules. Cell 154, 1151-1161 (2013).
12. Madak, J. T., Bankhead, A., 3rd, Cuthbertson, C. R., Showalter, H. D. & Neamati, N. Revisiting the role of dihydroorotate dehydrogenase as a therapeutic target for cancer. Pharmacol Ther 195, 111-131 (2019).
13. Arai, M. et al. Import into mitochondria of phospholipid hydroperoxide glutathione peroxidase requires a leader sequence. Biochem Biophys Res Commun 227, 433-439 (1996).
14. Trnka, J., Blaikie, F. H., Smith, R. A. & Murphy, M. P. A mitochondria-targeted nitroxide is reduced to its hydroxylamine by ubiquinol in mitochondria. Free Radic Biol Med 44, 1406-1419 (2008).
15. Prime, T. A. et al. A ratiometric fluorescent probe for assessing mitochondrial phospholipid peroxidation within living cells. Free Radic Blot Med 53, 544-553 (2012).
16. Shimada, K. et al. Global survey of cell death mechanisms reveals metabolic regulation of ferroptosis. Nature chemical biology 12, 497-503 (2016).
17. Hakkaart, G. A., Dassa, E. P., Jacobs, H. T. & Rustin, P. Allotopic expression of a mitochondrial alternative oxidase confers cyanide resistance to human cell respiration. EMBO Rep 7, 341-345 (2006).
18. Peters, G. J. et al. In vivo inhibition of the pyrimidine de novo enzyme dihydroorotic acid dehydrogenase by brequinar sodium (DUP-785; NSC 368390) in mice and patients. Cancer Res 50, 4644-4649 (1990).
19. Natale, R. et al. Multicenter phase II trial of brequinar sodium in patients with advanced melanoma. Ann Oncol 3, 659-660 (1992).
20. Urba, S. et al. Multicenter phase II trial of brequinar sodium in patients with advanced squamous-cell carcinoma of the head and neck. Cancer Chemother Pharmacol 31, 167-169 (1992).
21. Maroun, J. et al. Multicenter phase II study of brequinar sodium in patients with advanced lung cancer. Cancer Chemother Pharmacol 32, 64-66 (1993).
22. Lei, G. et al. The role of ferroptosis in ionizing radiation-induced cell death and tumor suppression. Cell research 30, 146-162 (2020).
23. Gout, P. W., Buckley, A. R., Simms, C. R. & Bruchovsky, N. Sulfasalazine, a potent suppressor of lymphoma growth by inhibition of the x(c)-cystine transporter: a new action for an old drug. Leukemia 15, 1633-1640 (2001).
24. Zou, Y. et al. A GPX4-dependent cancer cell state underlies the clear-cell morphology and confers sensitivity to ferroptosis. Nat Commun 10, 1617 (2019).
25. Soula, M. et al. Metabolic determinants of cancer cell sensitivity to canonical ferroptosis inducers. Nat Chem Biol 16, 1351-1360 (2020).
26. Gao, M. et al. Role of Mitochondria in Ferroptosis. Mol Cell 73, 354-363 e353 (2019).
27. Lee, H. et al. Energy-stress-mediated AMPK activation inhibits ferroptosis. Nature cell biology 22, 225-234 (2020).
28. Wang, W. et al. CD8(+) T cells regulate tumour ferroptosis during cancer immunotherapy. Nature 569, 270-274 (2019).
29. Lang, X. et al. Radiotherapy and immunotherapy promote tumoral lipid oxidation and ferroptosis via synergistic repression of SLC7A11. Cancer Discov (2019).
30. Ye, L. F. et al. Radiation-Induced Lipid Peroxidation Triggers Ferroptosis and Synergizes with Ferroptosis Inducers. ACS Chem Biol 15, 469-484 (2020).
31. Zhang, Y., Koppula, P. & Gan, B. Regulation of H2A ubiquitination and SLC7A11 expression by BAP1 and PRC1. Cell Cycle 18, 773-783 (2019).
32. Chauhan, A. S. et al. STIM2 interacts with AMPK and regulates calcium-induced AMPK activation. FASEB J 33, 2957-2970 (2019).
33. Liu, X. et al. Cystine transporter regulation of pentose phosphate pathway dependency and disulfide stress exposes a targetable metabolic vulnerability in cancer. Nat Cell Biol 22, 476-486 (2020).
34. Heinrich, P. et al. Correcting for natural isotope abundance and tracer impurity in MS-, MS/MS- and high-resolution-multiple-tracer-data from stable isotope labeling experiments with IsoCorrectoR. Sci Rep 8, 17910 (2018).
35. Koppula, P., Zhang, Y., Shi, J., Li, W. & Gan, B. The glutamate/cystine antiporter SLC7A11/xCT enhances cancer cell dependency on glucose by exporting glutamate. J Biol Chem 292, 14240-14249 (2017).
36. Liu, X. & Gan, B. lncRNA NBR2 modulates cancer cell sensitivity to phenformin through GLUT1. Cell Cycle 15, 3471-3481 (2016).
37. Dai, F. et al. BAP1 inhibits the ER stress gene regulatory network and modulates metabolic stress response. Proc Natl Acad Sci USA 114, 3192-3197 (2017).
38. Zhang, Y. et al. H2A Monoubiquitination Links Glucose Availability to Epigenetic Regulation of the Endoplasmic Reticulum Stress Response and Cancer Cell Death. Cancer Res 80, 2243-2256 (2020).
39. Fang, J. et al. Dihydro-orotate dehydrogenase is physically associated with the respiratory complex and its loss leads to mitochondrial dysfunction. Biosci Rep 33, e00021 (2013).
40. Nagase, M., Yamamoto, Y., Mitsui, J. & Tsuji, S. Simultaneous detection of reduced and oxidized forms of coenzyme Q10 in human cerebral spinal fluid as a potential marker of oxidative stress. J Clin Biochem Nutr 63, 205-210 (2018).
41. Zhang, Y. et al. Imidazole Ketone Erastin Induces Ferroptosis and Slows Tumor Growth in a Mouse Lymphoma Model. Cell Chem Biol (2019).
42. Liu, X. et al. LncRNA NBR2 engages a metabolic checkpoint by regulating AMPK under energy stress. Nat Cell Biol 18, 431-442 (2016).
43. Lee, H. et al. BAF180 regulates cellular senescence and hematopoietic stem cell homeostasis through p21. Oncotarget 7, 19134-19146 (2016).
44. Lin, A. et al. The FoxO-BNIP3 axis exerts a unique regulation of mTORC1 and cell survival under energy stress. Oncogene 33, 3183-3194 (2014).
45. Lin, A. et al. FoxO transcription factors promote AKT Ser473 phosphorylation and renal tumor growth in response to pharmacologic inhibition of the PI3K-AKT pathway. Cancer Res 74, 1682-1693 (2014).
46. Gan, B. et al. FoxOs enforce a progression checkpoint to constrain mTORC1-activated renal tumorigenesis. Cancer Cell 18, 472-484 (2010).
47. Gan, B. et al. Role of FIP200 in cardiac and liver development and its regulation of TNFalpha and TSC-mTOR signaling pathways. J Cell Biol 175, 121-133 (2006).

Having now fully described this invention, it will be understood by those of ordinary skill in the art that the same can be performed within a wide and equivalent range of conditions, formulations, and other parameters without affecting the scope of the invention or any aspect thereof.

Other aspects of the present disclosure will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

What is claimed is:

1. A method for treating cancer, the method comprising administering to a subject in need thereof a therapeutically effective amount of a dihydroorotate dehydrogenase (DHODH) inhibitor, wherein the cancer has an altered glutathione peroxidase 4 (GPX4) expression as compared to a control sample.

2. The method of claim 1, wherein the altered GPX4 expression is a low expression level of GPX4 as compared to a control sample.

3. The method of claim 1 or 2, wherein the cancer is a tumor.

4. The method of claim 3, wherein the tumor is a carcinoma.

5. The method of any one of claims 1 to 4, wherein the cancer is relapsed, refractory, or refractory following at least one prior therapy comprising administration of at least one anticancer agent.

6. The method of any one of claims 1 to 5, wherein the cancer is selected from fibrosarcoma, lung squamous cell carcinoma, lung adenocarcinoma, renal cell carcinoma, breast adenocarcinoma, colorectal adenocarcinoma, endocervical adenocarcinoma, or T acute lymphoblastic leukemia.

7. The method of any one of claims 1 to 6, wherein the DHODH inhibitor is selected from the group consisting of Ag-636, ASLAN003, BAY2402234, leflunomide, brequinar, teriflunomide, IMU-838, PP-001, PTC299, and combinations thereof.

8. The method of any one of claims 1 to 6, wherein the DHODH inhibitor is selected from the group consisting of leflunomide, brequinar, teriflunomide, and combinations thereof.

9. The method of claim 1, wherein the altered GPX4 expression is a high expression level of GPX4 as compared to a control sample.

10. The method of claim 9, wherein the cancer is a tumor.

11. The method of claim 9 or 10, wherein the tumor is a carcinoma.

12. The method of claim 9, wherein the cancer is relapsed, refractory, or refractory following at least one prior therapy comprising administration of at least one anticancer agent.

13. The method of claim 10, wherein the cancer is selected from fibrosarcoma, lung squamous cell carcinoma, lung adenocarcinoma, renal cell carcinoma, breast adenocarcinoma, colorectal adenocarcinoma, endocervical adenocarcinoma, or T acute lymphoblastic leukemia.

14. The method of any one of claims 9 to 13, wherein the method further comprises administering a therapeutically effective amount of a ferroptosis inducer.

15. The method of claim 14, wherein the ferroptosis inducer is a class I ferrotosis inducer, a class II ferroptosis inducer, or a combination thereof.

16. The method of claim 14 or 15, wherein the ferroptosis inducer is selected from the group consisting of sulfasalazine, rosiglitazone, rosiglitazone maleate, bardoxolone methyl, linagliptin, curcumin, zileuton, pioglitazone HCl, nordihydroguaiaretic acid (NDGA), troglitazone, setanaxib, deferoxamine mesylate, sorafenib tosylate, cisplatin, rosadustat, lapatinib, simvastatin, deferasirox, sorafenib, erastin, imidazole ketone erastin, RSL3, (1S,3R)-RSL3, ML210, ML162, and combinations thereof.

17. The method of any one of claims 14 to 16, wherein the ferroptosis inducer is selected from the group consisting of sulfasalazine, sorafenib tosylate, erastin, imidazole ketone erasin, and combinations thereof.

18. The method of any one of claims 1 to 17, wherein the subject is a human.

19. A method of treating a subject with a dihydroorotate dehydrogenase (DHODH) inhibitor, wherein the subject is afflicted with a cancer, the method comprising
a. determining, in the cancer sample, the expression level of glutathione peroxidase 4 (GPX4), and
b. if the expression level of GPX4 is low as compared to relative GPX4 expression level in a control sample, then administering a therapeutically effective amount of the DHODH inhibitor to the subject, or
c. if the expression level of GPX4 is high as compared to relative GPX4 expression level in a control sample, then administering a therapeutically effective amount of the DHODH inhibitor and a therapeutically effective amount of a ferroptosis inducer to the subject.

20. A method of treating a subject afflicted with a cancer, comprising administering to the subject a therapeutically effective amount of a dihydroorotate dehydrogenase (DHODH) inhibitor, wherein, prior to the administration, the subject is identified as exhibiting an altered expression level of glutathione peroxidase 4 (GPX4) as compared to relative GPX4 expression level in a control sample.

21. The method of claim 19 or 20, wherein if the subject is identified as exhibiting a low expression level of GPX4, the treatment comprises administering a therapeutically effective amount of the DHODH inhibitor to the subject.

22. The method of claim 19 or 20, wherein if the subject is identified as exhibiting a high expression level of GPX4, the treatment comprises administering a therapeutically effective amount of the DHODH inhibitor and a therapeutically effective amount of a ferroptosis inducer to the subject.

23. The method of any one of claims 20 to 22, wherein identifying the subject as exhibiting an altered expression level of GPX4 comprises obtaining a cancer sample from the subject and analyzing the sample for the GPX4 expression level.

24. A method of identifying a subject afflicted with a cancer as suitable for treatment with a dihydroorotate dehydrogenase (DHODH) inhibitor, the method comprising determining whether the subject has an altered expression level of glutathione peroxidase 4 (GPX4) as compared to relative GPX4 expression level in a control sample, wherein
a. if the expression level of GPX4 is low as compared to relative GPX4 expression level in a control sample, then a therapeutically effective amount of the DHODH inhibitor can be administered to the subject, or
b. if the expression level of GPX4 is high as compared to relative GPX4 expression level in a control sample, then a therapeutically effective amount of the DHODH inhibitor and a therapeutically effective amount of a ferroptosis inducer can be administered to the subject.

25. The method of claim 24, wherein determining whether the subject has an altered expression level of GPX4 comprises obtaining a cancer sample from the subject and analyzing the sample for the GPX4 expression level.

26. The method of claim 24 or 25, wherein the dihydroorotate dehydrogenase (DHODH) inhibitor is selected from the group consisting of Ag-636, ASLAN003, BAY2402234, leflunomide, brequinar, teriflunomide, IMU-838, PP-001, PTC299, and combinations thereof.

27. The method of any one of claims 24 to 26, wherein the dihydroorotate dehydrogenase (DHODH) inhibitor is selected from the group consisting of leflunomide, brequinar, teriflunomide, and combinations thereof.

28. The method of any one of claims 24 to 27, wherein the ferroptosis inducer is a class I ferrotosis inducer, a class II ferroptosis inducer, or a combination thereof.

29. The method of any one of claims 24 to 28, wherein the ferroptosis inducer is selected from the group consisting of sulfasalazine, rosiglitazone, rosiglitazone maleate, bardoxolone methyl, linagliptin, curcumin, zileuton, pioglitazone HCl, nordihydroguaiaretic acid (NDGA), troglitazone, setanaxib, deferoxamine mesylate, sorafenib tosylate, cisplatin, rosadustat, lapatinib, simvastatin, deferasirox, sorafenib, erastin, imidazole ketone erastin, RSL3, (1S,3R)-RSL3, ML210, ML162, and combinations thereof.

30. The method of any one of claims 24 to 29, wherein the ferroptosis inducer is selected from the group consisting of sulfasalazine, sorafenib tosylate, erastin, imidazole ketone erasin, and combinations thereof.

31. The method of any one of claims 19 to 30, wherein the subject is a human.

32. The method of any one of claims 19, 23, or 25 to 31, wherein the cancer sample comprises tumor tissue, intratumoral tissue, blood sample, bone marrow, or combinations thereof.

33. The method of any one of claims 19 to 32, wherein the GPX4 expression levels are determined using sequencing or any technology that measures RNA or protein expression level.

34. The method of claim 33, wherein the GPX4 expression levels are determined by PCR, real-time PCR, deep sequencing, Next Generation Sequencing (NGS), RNA-Seq, EdgeSeq, PCR, Nanostring, microarray expression profiling, immunohistochemical methods, ELISA, Western analysis, HPLC, proteomics assays, or a combination thereof.

35. The method of any one of claims 1 to 34, further comprising
a. administering chemotherapy;
b. performing surgery;
c. administering radiation therapy;
d. administering immunotherapy;
e. administering targeted therapy; or
f. any combination thereof.

36. The method of any one of claims 1 to 35, wherein the administering reduces the cancer burden.
